SEQUENCE LISTING (88 sequences)

SEQ ID NO: 1 - DNA, 1411 bp, Homo sapiens
ccttcgcaagccctcatttcaccaggcccccggcttggggcgccttccttccccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaa

SEQ ID NO: 2 - DNA, 1346 bp, Mus musculus
aaccgtccctaggtgagccgtctttccaccaggcccccggctcggggtgcccaccttccccatggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaactgaggcaccagccctccctggggatgctgtgagccaaggcaagggaggtagacaagagaacctggagctttggggttaaattcttttactgaggagggattaaaagcacaacaggggtggggggtgggatggggaaagaagctcagtgatgctgttgatcaggagcctggcctgtctgtcactcatcattttgttcttaaataaagactgggacacacagtagatagct

SEQ ID NO: 3 - DNA, 2518 bp, Homo sapiens
ctattaacttgttcaaaaaagtatcaggagttgtcaaggcagagaagagagtgtttgcaaaagggggaaagtagtttgctgcctctttaagactaggactgagagaaagaagaggagagagaaagaaagggagagaagtttgagccccaggcttaagcctttccaaaaaataataataacaatcatcggcggcggcaggatcggccagaggaggagggaagcgctttttttgatcctgattccagtttgcctctctctttttttcccccaaattattcttcgcctgattttcctcgcggagccctgcgctcccgacacccccgcccgcctcccctcctcctctccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaagaaaaaggaaaaaaaaaaatcccatcacccacagcaaatgacagctgcaaaagagaacaccaatcccatccacactcacgcaaaaaccgcgatgccgacaagaaaacttttatgagagagatcctggacttctttttgggggactatttttgtacagagaaaacctggggagggtggggagggcgggggaatggaccttgtatagatctggaggaaagaaagctacgaaaaactttttaaaagttctagtggtacggtaggagctttgcaggaagtttgcaaaagtctttaccaataatatttagagctagtctccaagcgacgaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgaaaagaagaaaattattcaaatttggacattttaattgtttaaaaattgtacaaaaggaaaaaattagaataagtactggcgaaccatctctgtggtcttgtttaaaaagggcaaaagttttagactgtactaaattttataacttactgttaaaagcaaaaatggccatgcaggttgacaccgttggtaatttataatagcttttgttcgatcccaactttccattttgttcagataaaaaaaaccatgaaattactgtgtttgaaatattttcttatggtttgtaatatttctgtaaatttattgtgatattttaaggttttcccccctttattttccgtagttgtattttaaaagattcggctctgtattatttgaatcagtctgccgagaatccatgtatatatttgaactaatatcatccttataacaggtacattttcaacttaagtttttactccattatgcacagtttgagataaataaatttttgaaatatggacactgaaaaaaaaaaaaaaaaaa

SEQ ID NO: 4 - DNA, 2457 bp, Mus musculus
ctattaacttgttcaaaaaagtatcaggagttgtcaaggcagagaagagagtgtttgcaaaaagggaaaagtactttgctgcctctttaagactagggctgggagaaagaagaggagagagaaagaaaggagagaagtttggagcccgaggcttaagcctttccaaaaactaatcacaacaatcgcggcggcccgaggaggagagcgcctgttttttcatcccaattgcacttcgcccgtctcgagctccgcttccccccaactattctccgccagatctccgcgcagggccgtgcacgccgaggcccccgcccgcggcccctgcatcccggcccccgagcgcggcccccacagtcccggccgggccgagggttggcggccgccggcgggccgcgcccgcccagcgcccgcatgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatgtgagggctggactgcgaactggagaaggggagagattttcaaagagatacaagggaattgggaggggtgcaaaaagaggagagtaggaaaaatctgataatgctcaaaaggaaaaaaaatctccgcagcgaaacgacagctgcggaaaaaaaccaccaatcccatccaaattaacgcaaaaaccgtgatgccgactagaaaacttttatgagagatcttgggacttctttttgggggactatttttgtacagagaaaacctgagggcggcggggagggcgggggaatcggaccatgtatagatctggaggaaaaaaactacgcaaaacttttttttaaagttctagtggtacgttaggcgcttcgcagggagttcgcaaaagtctttaccagtaatatttagagctagactccgggcgatgaaaaaaaagttttaatatttgcaagcaacttttgtacagtatttatcgagataaacatggcaatcaaatgtccattgtttataagctgagaatttgccaatatttttcgaggaaagggttcttgctgggttttgattctgcagcttaaatttaggaccgttacaaacaaggaaggagtttattcggatttgaacattttagttttaaaattgtacaaaaggaaaacatgagagcaagtactggcaagaccgttttcgtggtcttgtttaaggcaaacgttctagattgtactaaatttttaacttactgttaaaggcaaaaaaaaaatgtccatgcaggttgatatcgttggtaatttataatagcttttgttcaatcctaccctttcattttgttcacataaaaaatatggaattactgtgtttgaaatattttcttatggtttgtaatatttctgtaaattgtgatattttaaggtttttccccccttttattttccgtagttgtattttaaaagattcggctctgttattggaatcaggctgccgagaatccatgtatatatttgaactaataccatccttataacagctacattttcaacttaagtttttactccattatgcacagtttgagataaataaatttttgaaatatggacactgaaa

SEQ ID NO: 5 - DNA, 2121 bp, Homo sapiens
ctgctcgcggccgccaccgccgggccccggccgtccctggctcccctcctgcctcgagaagggcagggcttctcagaggcttggcgggaaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgcttgatcccccaggccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaa

SEQ ID NO: 6 - DNA, 2399 bp, Mus musculus
cccgcccacccgccctttatattccgggggtctgcgcggccgaggacccctgggctgcgctgctctcagctgccgggtccgactcgcctcactcagctcccctcctgcctcctgaagggcagggcttcgccgacgcttggcgggaaaaagaagggaggggagggatcctgagtcgcagtataaaagaagcttttcgggcgtttttttctgactcgctgtagtaattccagcgagagacagagggagtgagcggacggttggaagagccgtgtgtgcagagccgcgctccggggcgacctaagaaggcagctctggagtgagaggggctttgcctccgagcctgccgcccactctccccaaccctgcgactgacccaacatcagcggccgcaaccctcgccgccgctgggaaactttgcccattgcagcgggcagacacttctcactggaacttacaatctgcgagccaggacaggactccccaggctccggggagggaatttttgtctatttggggacagtgttctctgcctctgcccgcgatcagctctcctgaaaagagctcctcgagctgtttgaaggctggatttcctttgggcgttggaaaccccgcagacagccacgacgatgcccctcaacgtgaacttcaccaacaggaactatgacctcgactacgactccgtacagccctatttcatctgcgacgaggaagagaatttctatcaccagcaacagcagagcgagctgcagccgcccgcgcccagtgaggatatctggaagaaattcgagctgcttcccaccccgcccctgtccccgagccgccgctccgggctctgctctccatcctatgttgcggtcgctacgtccttctccccaagggaagacgatgacggcggcggtggcaacttctccaccgccgatcagctggagatgatgaccgagttacttggaggagacatggtgaaccagagcttcatctgcgatcctgacgacgagaccttcatcaagaacatcatcatccaggactgtatgtggagcggtttctcagccgctgccaagctggtctcggagaagctggcctcctaccaggctgcgcgcaaagacagcaccagcctgagccccgcccgcgggcacagcgtctgctccacctccagcctgtacctgcaggacctcaccgccgccgcgtccgagtgcattgacccctcagtggtctttccctacccgctcaacgacagcagctcgcccaaatcctgtacctcgtccgattccacggccttctctccttcctcggactcgctgctgtcctccgagtcctccccacgggccagccctgagcccctagtgctgcatgaggagacaccgcccaccaccagcagcgactctgaagaagagcaagaagatgaggaagaaattgatgtggtgtctgtggagaagaggcaaacccctgccaagaggtcggagtcgggctcatctccatcccgaggccacagcaaacctccgcacagcccactggtcctcaagaggtgccacgtctccactcaccagcacaactacgccgcacccccctccacaaggaaggactatccagctgccaagagggccaagttggacagtggcagggtcctgaagcagatcagcaacaaccgcaagtgctccagccccaggtcctcagacacggaggaaaacgacaagaggcggacacacaacgtcttggaacgtcagaggaggaacgagctgaagcgcagcttttttgccctgcgtgaccagatccctgaattggaaaacaacgaaaaggcccccaaggtagtgatcctcaaaaaagccaccgcctacatcctgtccattcaagcagacgagcacaagctcacctctgaaaaggacttattgaggaaacgacgagaacagttgaaacacaaactcgaacagcttcgaaactctggtgcataaactgacctaactcgaggaggagctggaatctctcgtgagagtaaggagaacggttccttctgacagaactgatgcgctggaattaaaatgcatgctcaaagcctaacctcacaaccttggctggggctttgggactgtaagcttcagccataattttaactgcctcaaacttaaatagtataaaagaacttttttttatgcttcccatcttttttctttttccttttaacagatttgtatttaattgtttttttaaaaaaatcttaaaatctatccaattttcccatgtaaatagggccttgaaatgtaaataactttaataaaacgtttataacagttacaaaagattttaagacatgtaccataattttttttatttaaagacattttcatttttaaagttgatttttttctattgtttttagaaaaaaataaaataattggaaaaaatac

SEQ ID NO: 7 - DNA, 2639 bp, Homo sapiens
tcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaacctataatattttatctg

SEQ ID NO: 8 - DNA, 2756 bp, Mus musculus
cgtggccgcgacaacggtgggggacactgctgagtccaagagcgtgcagcctggccatcggacctacttatctgccttgctgattgtctatttttataagagtttacaacttttctaagaatttttgtatacaaaggaacttttttaaagacatcgccggtttatattgaatccaaagaaggatctcgggcaatctgggggttttggtttgaggttttgtttctaaagtttttaatcttcgttgactttggggctcgggtacccctctctcttcttcggactccggaggaccttctgggcccccacattaatgaggcagccacctggcgagtctgacatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccagcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctcggatgaactgaccaggcactaccgcaaacacacagggcaccggccctttcagtgccagaagtgcgacagggccttttccaggtcggaccaccttgccttacacatgaagaggcacttttaaatcccacgtagtggatgtgacccacactgccaggagagagagttcagtatttttttttctaacctttcacactgtcttcccacgaggggaggagcccagctggcaagcgctacaatcatggtcaagttcccagcaagtcagcttgtgaatggataatcaggagaaaggaagagtccaagagacaaaacagaaatactaaaaacaaacaaacaaaaaaacaaacaaaaaaaaacaaaagaaaaaaatcacagaacagatggggtctgagactggatggatcttctatcattccaataccaaatccaacttgaacatgcccggacttacaaaatgccaaggggtgactggaagtttgtggatatcagggtatacactaaatcagtgagcttggggggagggaagaccaggattcccttgaattgtgtttcgatgatgcaatacacacgtaaagatcaccttatatgctctttgccttctaaaaaaaaaagccattattgtgtcggaggaagaggaagcgattcaggtacagaacatgttctaacagcctaaatgatggtgcttggtgagttgtggtcctaaaggtaccaaacgggggagccaaagttctccaactgctgcatacttttgacaaggaaaatctagttttgtcttccgatctacattgatgacctaagccaggtaaataagcctggtttatttctgtaacatttttatgcagacagtctgttatgcactgtggtttcagatgtgcaataatttgtacaatggtttattcccaagtatgcctttaagcagaacaatgtgttttctatatagttccttgccttaataaatatgtaatataaatttaagcaaacttctattttgtatatttgtaaactacaaagtaaaaaaaaatgaacattttgtggagtttgtattttgcatactcaaggtgagaaataagttttaaataaacctataatattttatctgaaaaaaaaaaaaaaaaaag

SEQ ID NO: 9 - DNA, 20 bp, Artificial Sequence
  synthetic oligonucleotide
tgaagtgtgacgtggacatc

SEQ ID NO: 10 - DNA, 20 bp, Artificial Sequence
  synthetic oligonucleotide
ggaggagcaatgatcttgat

SEQ ID NO: 11 - DNA, 20 bp, Artificial Sequence
  synthetic oligonucleotide
agcgaaccagtatcgagaac

SEQ ID NO: 12 - DNA, 20 bp, Artificial Sequence
  synthetic oligonucleotide
ttacagaaccacactcggac

SEQ ID NO: 13 - DNA, 20 bp, Artificial Sequence
  synthetic oligonucleotide
tgaacctcagctacaaacag

SEQ ID NO: 14 - DNA, 20 bp, Artificial Sequence
  synthetic oligonucleotide
tggtggtaggaagagtaaag

SEQ ID NO: 15 - DNA, 20 bp, Artificial Sequence
  synthetic oligonucleotide
gtcatcacaacagcagttct

SEQ ID NO: 16 - DNA, 20 bp, Artificial Sequence
  synthetic oligonucleotide
gactactaaggacacatgca

SEQ ID NO: 17 - DNA, 4018 bp, Homo sapiens
caggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 18 - DNA, 2119 bp, Simian virus 40
agttttaaacagagaggaatctttgcagctaatggaccttctaggtcttgaaaggagtgcctgggggaatattcctctgatgagaaaggcatatttaaaaaaatgcaaggagtttcatcctgataaaggaggagatgaagaaaaaatgaagaaaatgaatactctgtacaagaaaatggaagatggagtaaaatatgctcatcaacctgactttggaggcttctgggatgcaactgagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatccattttctgttattgaggaaagtttgccaggtgggttaaaggagcatgattttaatccagaagaagcagaggaaactaaacaagtgtcctggaagcttgtaacagagtatgcaatggaaacaaaatgtgatgatgtgttgttattgcttgggatgtacttggaatttcagtacagttttgaaatgtgtttaaaatgtattaaaaaagaacagcccagccactataagtaccatgaaaagcattatgcaaatgctgctatatttgctgacagcaaaaaccaaaaaaccatatgccaacaggctgttgatactgttttagctaaaaagcgggttgatagcctacaattaactagagaacaaatgttaacaaacagatttaatgatcttttggataggatggatataatgtttggttctacaggctctgctgacatagaagaatggatggctggagttgcttggctacactgtttgttgcccaaaatggattcagtggtgtatgactttttaaaatgcatggtgtacaacattcctaaaaaaagatactggctgtttaaaggaccaattgatagtggtaaaactacattagcagctgctttgcttgaattatgtggggggaaagctttaaatgttaatttgcccttggacaggctgaactttgagctaggagtagctattgaccagtttttagtagtttttgaggatgtaaagggcactggaggggagtccagagatttgccttcaggtcagggaattaataacctggacaatttaagggattatttggatggcagtgttaaggtaaacttagaaaagaaacacctaaataaaagaactcaaatatttccccctggaatagtcaccatgaatgagtacagtgtgcctaaaacactgcaggccagatttgtaaaacaaatagattttaggcccaaagattatttaaagcattgcctggaacgcagtgagtttttgttagaaaagagaataattcaaagtggcattgctttgcttcttatgttaatttggtacagacctgtggctgagtttgctcaaagtattcagagcagaattgtggagtggaaagagagattggacaaagagtttagtttgtcagtgtatcaaaaaatgaagtttaatgtggctatgggaattggagttttagattggctaagaaacagtgatgatgatgatgaagacagccaggaaaatgctgataaaaatgaagatggtggggagaagaacatggaagactcagggcatgaaacaggcattgattcacagtcccaaggctcatttcaggcccctcagtcctcacagtctgttcatgatcataatcagccataccacatttgtagaggttttacttgctttaaaaaacctcccacacctccccctgaacctgaaacataa

SEQ ID NO: 19 - DNA, 3580 bp, Artificial Sequence
  synthetic construct
atggataaaccatggattacaaggatgacgacgataagatcgcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactggaacaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctggaaaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaaccagctgttgaattttgaccttcttaagcttgcgggagacgtcgagtccaaccctgggcccatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaaggcctgggcgccgagtggaaacttttgtcggagacggagaagcggccgttcatcgacgaggctaagcggctgcgagcgctgcacatgaaggagcacccggattataaataccggccccggcggaaaaccaaacgctcatgaagaaggataagtacacgctgcccggcgggctgctggcccccggcggcaatagcatggcgagcggggtcggggtgggcgccggcctgggcgcgggcgtgaaccagcgcatggacagttacgcgacatgaacggctggagcaacggcagctacagcatgatgcaggaccagctgggctacccgcagcacccgggcctcaatgcgcacggcgcagcgcagatgcagcccatgcaccgctacgacgtgagcgccctgagtacaactccatgaccagctcgcagacctacatgaacggctcgcccacctacagcatgtcctactcgcagcagggcacccctggcatggctcttggctccatgggttcggtggtcaagtccgaggccagcccagcccccctgtggttacctcttcctcccactccagggcgccctgccaggccggggacctccgggacatgatcagcatgtatctccccggcgccgaggtgccggaacccgccgcccccagcagacttcacatgtccagcactaccagagcggcccggtgcccggcacggccattaacggcacactgcccctctcacacatggagggcagaggaagtctgctaacatgcggtgacgtcgaggagaatcctggcccaatggctgtcagcgacgcgctgctcccatctttctccacgttcgcgtctggcccggcgggaagggagaagacactgcgtcaagcaggtgccccgaataaccgctggcgggaggagctctcccacatgagcgacttcccccagtgcttcccggccgcccctatgacctggcggcggcgaccgtggccacagacctggagagcggcggagccggtgcggcttgcggcggtagcaacctggcgcccctacctcggagagagaccgagagttcaacgatctcctggacctggactttattctctccaattcgctgacccatcctccggagtcagtggccgccaccgtgtcctcgtcagcgtcagcctcctcttcgtcgtcgccgtcgagcagcggccctccagcgcgccctccacctgcagcttcacctatccgatccgggccgggaacgacccgggcgtggcgccgggcggcacgggcggaggcctcctctatggcagggagtccgctccccctccgacggctcccttcaacctggcggcatcaacgacgtgagcccctcgggcggcttcgtggccgagctcctgcggccagaattggacccggtgtacattccgccgcagcagccgcagccgccaggtggcgggctgatgggcaagttcgtgctgaaggcgtgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcggaggcggtctcttcgtgcacccacttgggcgctggaccccctctcagcaatggccaccggccggctgcacacgacttccccctggggcggcagctccccagcaggactaccccgaccctgggtcttgagaagtgctgagcagcagggactgtcaccctgccctgccgcttcctcccggcttccatccccacccggggcccaattacccatccttcctgcccgatcagatgcagccgcaagtcccgccgctccattaccaagagctatgccacccggttcctgcatgccagaggagcccaagccaaagaggggaagacgatcgtggccccggaaaaggaccgccacccacacttgtgattacgcgggctgcggcaaaacctacacaaagagttcccatctcaagcacacctgcgaacccacacaggtgagaaaccttaccactgtgactgggacggctgtggatggaaattcgcccgctcagatgaactgaccaggcactaccgtaaacacacggggcaccgcccgttccagtgcaaaaatgcgaccgagcattttccaggtcggaccacctcgccttacacatgaagaggcatttttaa

SEQ ID NO: 20 - DNA, 4985 bp, Artificial Sequence
  synthetic construct
accatggattacaaggatgacgacgataagatcgcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactggaacaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctggaaaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaaccagctgttgaattttgaccttcttaagcttgcgggagacgtcgagtccaaccctgggcccatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcggcagcggcgcaagatggcccaggagaaccccaagatgcacaactcggagatcagcaagcgcctgggcgccgagtggaaacttttgtcggagacggagaagcggccgttcatcgacgaggctaagcggctgcgaggctgcacatgaaggagcacccggattataaataccggccccggcggaaaaccaagacgctcatgaagaaggataagtacacgctgcccggcgggctgctggcccccggcggcaatagcatggcgagcggggtcgggggggcgccggcctgggcgcgggcgtgaaccagcgcatggacagttacgcgcacatgaacggctggagcaacggcagctacagcatgatgcaggaccagctgggctacccgcagcacccgggcctcaatgcgcacggccagcgcagatgcagcccatgcaccgctacgacgtgagcgccctgcagtacaactccatgaccagctcgcagacctacatgaacggctcgcccacctacagcatgtcctactcgcagcagggcacccctggcatggccttggctccatgggttcggtggtcaagtccgaggccagctccagcccccctgtggttacctcttcctcccactccagggcgccctgccaggccggggacctccgggacatgatcagcatgtatctccccggcgccgaggtgccgaacccgccgcccccagcagacttcacatgtcccagcactaccagagcggcccggtgcccggcacggccattaacggcacactgcccctctcacacatggagggcagaggaagtctgctaacatgcggtgacgtcgaggagaatcctggcccaatggctgtcagcgacgcgctgctcccatctttctccacgttcgcgtctggcccggcgggaagggagaagacactgcgtcaagcaggtgccccgaataaccgctggcgggagggctctcccacatgaagcgacttcccccagtgcttcccggccgcccctatgacctggcggcggcgaccgtggccacagacctggagagcggcggagccggtgcggcttgcggcggtagcaacctggcgcccctacccggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcgcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgcgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggttcttcgtgcacccacttgggcgctggaccccctctcagcaatggccaccggccggctgcacacgacttccccctggggcggcagctccccagcaggactaccccgaccctgggtcttgaggaagtgctgagcagcaggactgtcaccctgccctgccgcttcctcccggcttccatccccacccggggcccaattacccatccttcctgcccgatcagatgcagccgcaagtcccgccgctccattaccaagagctcatgccacccggttcctgcatgcagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaccttaccactgtgactgggacggctgtggatggaaattcgcccgctcagatgaactgaccaggcactaccgtaaacacacggggcaccgcccgttccagtgccaaaaatgcgaccgagcattttccaggtcggacacctcgccttacacatgaagaggcattttcaatgtactaactacgctttgttgaaactcgctggcgatgttgaaagtaaccccggtcctctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtgcgggagcttctccacggccgaccagctggagatggtgaccgagctgctgggaggagacatggtgaaccagagtttcatctgcgacccggacgacgagaccttcatcaaaaacatcatcatccaggactgtatgtggagcggctttcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcccagagtgcatcgacccctcggtggtcttcccctaccctctcaacgacagcagctcgcccaagtcctgcgcctcgcaagactccagcgccttctctccgtcctcggattctctgctctcctcgacggagtcctccccgcaggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcacttctgctggaggccacagcaaacctcctcacagcccactggtcctcaagaggtgccacgtctccacacatcagcacaactacgcagcgcctccctccactcggaaggactatcctgctgccaagagggtcaagttggacgtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgcctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaacgacgagaacagttgaaacacaaacttgaacagctacggaactcttgtgcgtaa

SEQ ID NO: 21 - DNA, 3775 bp, Artificial Sequence
  synthetic construct
accatggattacaaggatgacgacgataagatcgcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactggaacaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctggaaaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaaccagctgttgaattttgaccttcttaagcttgcgggagacgtcgagtccaaccctgggcccatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccgcgccgaggtgccggaacccgccgcccccagcagacttcacatgtcccagcactaccagagcggcccggtgcccggcacggccattaacggcacactgcccctctcacacatggagggcagaggaagtctgctaacatgcggtgacgtcgaggagaatcctggcccaatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacaccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcgaccactaggtattttagtactccacaaaccatggatttattcctaaactactccatgaacatgcaacctgaagacgtgcaatgtactaactacgctttgttgaaactcgctggcgatgttgaaagtaaccccggtcctatgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttggcaccagagtaagctgcacatggaagggttccggagcttgaaggagggtgaggcagtggagttcacctttaagaagtcagccaagggtctggaatccatccgtgtcaccggacctggtggagtattctgattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattga

SEQ ID NO: 22 - protein, 22 aa, Foot-and-mouth disease virus
VKQTLNFDLLKLAGDVESNPGP

SEQ ID NO: 23 - protein, 20 aa, Equine rhinitis A virus
QCTNYALLKLAGDVESNPGP

SEQ ID NO: 24 - protein, 18 aa, Thosea asigna virus
EGRGSLLTCGDVEENPGP

SEQ ID NO: 25 - protein, 19 aa, Porcine teschovirus-1
ATNFSLLKQAGDVEENPGP

SEQ ID NO: 26 - DNA, 2098 bp, Homo sapiens
attataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtgaagatgagtgaaactgatattactcaatttcagtctggacactggctgaatccttcctctcccctcctcccatccctcataggatttttcttgtttggaaaccacgtgttctggtttccatgatgcccatccagtcaatctcatggagggtggagtatggttggagcctaatcagcgaggtttctttttttttttttttcctattggatcttcctggagaaaatactttttttttttttttttttgaaacggagtcttgctctgtcgcccaggctggagtgcagtggcgcggtcttggctcactgcaagctccgtctcccgggttcacgccattctcctgcctcagcctcccgagcagctgggactacaggcgcccgccacctcgcccggctaatattttgtatttttagtagagacggggtttcactgtgttagccaggatggtctcgatctcctgaccttgtgatccacccgcctcggcctccctaacagctgggatttacaggcgtgagccaccgcgccctgcctagaaaagacattttaataaccttggctgccgtctctggctatagataagtagatctaatactagtttggatatctttagggtttagaatctaacctcaagaataagaaatacaagtacaaattggtgatgaagatgtattcgtattgtttgggattgggaggctttgcttattttttaaaaactattgaggtaaagggttaagctgtaacatacttaattgatttcttaccgtttttggctctgttttgctatatcccctaatttgttggttgtgctaatctttgtagaaagaggtctcgtatttgctgcatcgtaatgacatgagtactgctttagttggtttaagttcaaatgaatgaaacaactatttttcctttagttgattttaccctgatttcaccgagtgtttcaatgagtaaatatacagcttaaacat

SEQ ID NO: 27 - DNA, 1356 bp, Mus musculus
tctatcgccttgagccgttggccttcagataggctgatttggttggtgtcttgctctttctgtgggaaggctgcggctcacttccttctgacttcttgataattttgcattagacatttaactcttctttctatgatctttccttctagacactgagttttttggttgttgcctaaaaccttttcagaaatcccttccctcgccatcacactgacatgagtgtgggtcttcctggtccccacagtttgcctagttctgaggaagcatcgaattctgggaacgcctcatcaatgcctgcagtttttcatcccgagaactattcttgcttacaagggtctgctactgagatgctctgcacagaggctgcctctcctcgcccttcctctgaagacctgcctcttcaaggcagccctgattcttctaccagtcccaaacaaaagctctcaagtcctgaggctgacaagggccctgaggaggaggagaacaaggtccttgccaggaagcagaagatgcggactgtgttctctcaggcccagctgtgtgcactcaaggacaggtttcagaagcagaagtacctcagcctccagcagatgcaagaactctcctccattctgaacctgagctataagcaggttaagacctggtttcaaaaccaaaggatgaagtgcaagcggtggcagaaaaaccagtggttgaagactagcaatggtctgattcagaagggctcagcaccagtggagtatcccagcatccattgcagctatccccagggctatctggtgaacgcatctggaagcctttccatgtggggcagccagacttggaccaacccaacttggagcagccagacctggaccaacccaacttggaacaaccagacctggaccaacccaacttggagcagccaggcctggaccgctcagtcctggaacggccagccttggaatgctgctccgctccataacttcggggaggactttctgcagccttacgtacagttgcagcaaaacttctctgccagtgatttggaggtgaatttggaagccactagggaaagccatgcgcattttagcaccccacaagccttggaattattcctgaactactctgtgactccaccaggtgaaatatgagacttacgcaacatctgggcttaaagtcagggcaaagccaggttccttccttcttccaaatattttcatattttttttaaagatttatttattcattatatgtaagtacactgtagctgtcttcagacactccagaagagggcgtcagatcttgttacgtatggttgtgagccaccatgtggttgctgggatttgaactcctgaccttcggaagagcagtcgg

SEQ ID NO: 28 - DNA, 3452 bp, Homo sapiens
cctttgccttcggacttctccggggccagcagccgcccgaccaggggcccggggccacgggctcagccgacgaccatgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattgagccacaatgggtgggggctattcttttgctatcaggaagttttgaggagcaggcagagtggagaaagtgggaatagggtgcattggggctagttggcactgccatgtatctcaggcttgggttcacaccatcaccctttcttccctctaggtggggggaaagggtgagtcaaaggaactccaaccatgctctgtccaaatgcaagtgagggttctgggggcaaccaggaggggggaatcaccctacaacctgcatactttgagtctccatccccagaatttccagcttttgaaagtggcctggatagggaagttgttttccttttaaagaaggatatataataattcccatgccagagtgaaatgattaagtataagaccagattcatggagccaagccactacattctgtggaaggagatctctcaggagtaagcattgtttttttttcacatcttgtatcctcatacccacttttgggatagggtgctggcagctgtcccaagcaatgggtaatgatgatggcaaaaagggtgtttgggggaacagctgcagacctgctgctctatgctcacccccgccccattctgggccaatgtgattttatttatttgctcccttggatactgcaccttgggtcccactttctccaggatgccaactgcactagctgtgtgcgaatgacgtatcttgtgcattttaactttttttccttaatataaatattctggttttgtatttttgtatattttaatctaaggccctcatttcctgcactgtgttctcaggtacatgagcaatctcagggatagccagcagcagctccaggtctgcgcagcaggaattactttttgttgtttttgccaccgtggagagcaactatttggagtgcacagcctattgaactacctcatttttgccaataagagctggcttttctgccatagtgtcctcttgaaaccccctctgccttgaaaatgttttatgggagactaggttttaactgggtggccccatgacttgattgccttctactggaagattgggaattagtctaaacaggaaatggtggtacacagaggctaggagaggctgggcccggtgaaaaggccagagagcaagccaagattaggtgagggttgtctaatcctatggcacaggacgtgctttacatctccagatctgttcttcaccagattaggttaggcctaccatgtgccacagggtgtgtgtgtgtttgtaaaactagagttgctaaggataagtttaaagaccaatacccctgtacttaatcctgtgctgtcgagggatggatatatgaagtaaggtgagatccttaacctttcaaaattttcgggttccagggagacacacaagcgagggttttgtggtgcctggagcctgtgtcctgccctgctacagtagtgattaatagtgtcatggtagctaaaggagaaaaagggggtttcgtttacacgctgtgagatcaccgcaaacctaccttactgtgttgaaacgggacaaatgcaatagaacgcattgggtggtgtgtgtctgatcctgggttcttgtctcccctaaatgctgccccccaagttactgtatttgtctgggctttgtaggacttcactacgttgattgctaggtggcctagtttgtgtaaatataatgtattggtctttctccgtgttctttgggggttttgtttacaaacttctttttgtattgagagaaaaatagccaaagcatctttgacagaaggttctgcaccaggcaaaaagatctgaaacattagtttggggggccctcttcttaaagtggggatcttgaaccatcctttcttttgtattccccttcccctattacctattagaccagatcttctgtcctaaaaacttgtcttctaccctgccctcttttctgttcacccccaaaagaaaacttacacacccacacacatacacatttcatgcttggagtgtctccacaactcttaaatgatgtatgcaaaaatactgaagctaggaaaaccctccatcccttgttcccaacctcctaagtcaagaccattaccatttctttctttcttttttttttttttttaaaatggagtctcaccgagaggcagaggttgcagtgagctgagatcgcaccactgcactccagcctggttacagagcaagactctgtctcaaacaaaacaaaacaaaacaaaaacacactactgtattttggatggatcaaacctccttaattttaatttctaatcctaaagtaaagagatgcaattgggggccttccatgtagaaagtggggtcaggaggccaagaaagggaatatgaatgtatatccaagtcactcaggaacttttatgcaggtgctagaaactttatgtcaaagtggccacaagattgtttaataggagacgaacgaatgtaactccatgtttactgctaaaaaccaaagctttgtgtaaaatcttgaatttatggggcgggagggtaggaaagcctgtacctgtctgtttttttcctgatccttttccctcattcctgaactgcaggagactgagcccctttgggctttggtgaccccatcactggggtgtgtttatttgatggttgattttgctgtactgggtacttcctttcccattttctaatcattttttaacacaagctgactcttcccttcccttctcctttccctgggaaaatacaatgaataaataaagacttattggtacgc

SEQ ID NO: 29 - DNA, 3480 bp, Mus musculus
cctttgcctccggacttctctggggccagcagccgcccgacctggggcccggggccacgggctcagcagacgaccatgggctcggtgtccaaccagcagtttgcaggtggctgcgccaaggcagcggagaaggcgccagaggaggcgccgcctgacgcggcccgagcggcagacgagccgcagctgctgcacggggccggcatctgtaagtggttcaacgtgcgcatggggttcggcttcctgtctatgaccgcccgcgctggggtcgcgctcgaccccccggtggacgtctttgtgcaccagagcaagctgcacatggaagggttccgaagcctcaaggagggtgaggcggtggagttcacctttaagaagtctgccaagggtctggaatccatccgtgtcactggccctggtggtgtgttctgtattgggagtgagcggcggccaaaagggaagaacatgcagaagcgaagatccaaaggagacaggtgctacaactgcggtgggctagaccatcatgccaaggaatgcaagctgccaccccagcccaagaagtgccacttttgccaaagcatcaaccatatggtggcctcgtgtccactgaaggcccagcagggccccagttctcagggaaagcctgcctacttccgggaggaagaggaagagatccacagccctgccctgctcccagaagcccagaattgaggcccaggagtcagggttattctttggctaatggggagtttaaggaaagaggcatcaatctgcagagtggagaaagtgggggtaagggtgggttgcgtgggtagcttgcactgccgtgtctcaggccggggttcccagtgtcaccctgtctttccttggagggaaggaaaggatgagacaaaggaactcctaccacactctatctgaaagcaagtgaaggcttttgtggggaggaaccaccctagaacccgaggctttgccaagtggctgggctagggaagttcttttgtagaaggctgtgtgatatttcccttgccagacgggaagcgaaacaagtgtcaaaccaagattactgaacctacccctccagctactatgttctggggaagggactcccaggagcagggcgaggttattttcacaccgtgcttattcataaccctgtcctttggtgctgtgctgggaatggtctctagcaacgggttgtgatgacaggcaaagagggtggttggggagacaactgctgacctgctgcccacacctcactcccagccctttctgggccaatgggattttaatttatttgctcccttaggtaactgcaccttgggtcccactttctccaggatgccaactgcactatctacgtgcgaatgacgtatcttgtgcgttttttttttttttaatttttaaaattttttttcatcttcttaatataaataatgggtttgtatttttgtatattttaatcttaaggccctcattcctgcactgtgttctcaggtacatgagcaatctcagggataataagtccgtagcagctccaggtctgctcagcaggaatactttgttttgttttgttttgatcaccatggagaccaaccatttggagtgcacagcctgttgaactacctcatttttgccgattacagctggcttttctgccatagcgtccttgaaaaatgtgtctcacgggtttcgattgagctgccccaagacttgatctggatttggcaaaacataggacatcactctaaacaggaaagggtggtacagagacattaaaaggctgggccaggtgaaaggcacaagaggaactttccataccagatccatccttttgccagattagtggaagcctgccatgcacagcagggtgtgagagagagagtgtgtatgtatgtgtgtgtggattttttttaatgcaaatttatgaagacgaggtgggttttgtttatttgattgctttttgtgctggggatggaatcttgggcttcatttgtgctaggaagtacactgccactgagttatcccagtaagaatgcaacttaagaccagtacccttattcccacactgtgctgtccaggcatgggaacatgaggcagggactcaactccttagcctttcacaatcttggctttctgagagactcatgagtatgggcctcagtggcaagtgtcctgccctgctgtagcgtgatggttgatagctaaaggaaagagggggtggggagtttcgtttacatgctttgagatcgccacaaacctacctcactgtgttgaaacgggacaaatgcaatagaacacattgggtggtgtgtgtgtgtgtctgatcttggtttcttgtctccctctccccccaaatgctgccctcacccctagttaattgtattcgtctggcctttgtaggacttttactgtctctgagttggtgattgctaggtggcctagttgtgtaaatataaatgtgttggtcttcatgttcttttggggttttattgtttacaaaacttttgttgtattgagagaaaaatagccaaagcatctttgacagaaagctctgcaccagacaacaccatctgaaacttaaatgtgcggtcctcttctcaaagtgaacctctgggaccatggcttatccttacctgttcctcctgtgtctcccattctggaccacagtgaccttcagacagcccctcttctccctcgtaagaaaacttaggctcatttacttctttgagcatctctgtaactcttgaaggacccatgtgaaaattctgaagaagccaggaacctcattctttccttgtccctaactcagtgaagagttttggttggtggttttgagacagggcctcactctgtagctggagatagagagcctcgggttcctggctctcctcctgccttctgcacagagtcccctgtgcagggattgcaggtgccgcttctccctggcaagaccatttatttcatggtgtgattcgcctttggatggatcaaaccaatgtaatctgtcacccttaggtcgagagaagcaattgtggggccttccatgtagaaagttggaatctggacaccagaaaagggactatgaatgtacagtgagtcactcaggaacttaatgccggtgcaagaaacttatgtcaaagaggccacaagattgttactaggagacggacgaatgtatctccatgtttactgctagaaaccaaagctttgtgagaaatcttgaatttatggggagggtgggaaagggtgtacttgtctgtcctttccccatctctttcctgaactgcaggagactaaggccccccaccccccggggcttggatgacccccacccctgcctggggtgttttatttcctagttgatttttactgtacccgggcccttgtattcctatcgtataatcatcctgtgacacatgctgacttttccttcccttctcttccctgggaaaataaagacttattggtactccagagttggtactg

SEQ ID NO: 30 - DNA, 20 bp, Artificial Sequence
  synthetic oligonucleotide
tgaagtgtgacgtggacatc

SEQ ID NO: 31 - DNA, 20 bp, Artificial Sequence
  synthetic oligonucleotide
ggaggagcaatgatcttgat

SEQ ID NO: 32 - DNA, 20 bp, Artificial Sequence
  synthetic oligonucleotide
agcgaaccagtatcgagaac

SEQ ID NO: 33 - DNA, 20 bp, Artificial Sequence
  synthetic oligonucleotide
ttacagaaccacactcggac

SEQ ID NO: 34 - DNA, 20 bp, Artificial Sequence
  synthetic oligonucleotide
agctacagcatgatgcagga

SEQ ID NO: 35 - DNA, 20 bp, Artificial Sequence
  synthetic oligonucleotide
ggtcatggagttgtactgca

SEQ ID NO: 36 - DNA, 20 bp, Artificial Sequence
  synthetic oligonucleotide
tgaacctcagctacaaacag

SEQ ID NO: 37 - DNA, 20 bp, Artificial Sequence
  synthetic oligonucleotide
tggtggtaggaagagtaaag

SEQ ID NO: 38 - DNA, 20 bp, Artificial Sequence
  synthetic oligonucleotide
actctgaggaggaacaagaa

SEQ ID NO: 39 - DNA, 19 bp, Artificial Sequence
  synthetic oligonucleotide
tggagacgtggcacctctt

SEQ ID NO: 40 - DNA, 20 bp, Artificial Sequence
  synthetic oligonucleotide
tctcaaggcacacctgcgaa

SEQ ID NO: 41 - DNA, 20 bp, Artificial Sequence
  synthetic oligonucleotide
tagtgcctggtcagttcatc

SEQ ID NO: 42 - DNA, 20 bp, Artificial Sequence
  synthetic oligonucleotide
tgtgcaccaacatctacaag

SEQ ID NO: 43 - DNA, 20 bp, Artificial Sequence
  synthetic oligonucleotide
gcgttcttggctttcaggat

SEQ ID NO: 44 - DNA, 20 bp, Artificial Sequence
  synthetic oligonucleotide
tcgctgagctgaaacaaatg

SEQ ID NO: 45 - DNA, 20 bp, Artificial Sequence
  synthetic oligonucleotide
cccttcttgaaggtttacac

SEQ ID NO: 46 - DNA, 20 bp, Artificial Sequence
  synthetic oligonucleotide
aaatgtttgtgttgcggtca

SEQ ID NO: 47 - DNA, 20 bp, Artificial Sequence
  synthetic oligonucleotide
tctggcacaggtgtcttcag

SEQ ID NO: 48 - DNA, 20 bp, Artificial Sequence
  synthetic oligonucleotide
cctcacttcactgcactgta

SEQ ID NO: 49 - DNA, 20 bp, Artificial Sequence
  synthetic oligonucleotide
caggttttctttccctagct

SEQ ID NO: 50 - DNA, 20 bp, Artificial Sequence
  synthetic oligonucleotide
cctcacttcactgcactgta

SEQ ID NO: 51 - DNA, 20 bp, Artificial Sequence
  synthetic oligonucleotide
ccttgaggtaccagagatct

SEQ ID NO: 52 - DNA, 19 bp, Artificial Sequence
  synthetic oligonucleotide
cccagcagacttcacatgt

SEQ ID NO: 53 - DNA, 20 bp, Artificial Sequence
  synthetic oligonucleotide
cctcccatttccctcgtttt

SEQ ID NO: 54 - DNA, 19 bp, Artificial Sequence
  synthetic oligonucleotide
cccagcagacttcacatgt

SEQ ID NO: 55 - DNA, 20 bp, Artificial Sequence
  synthetic oligonucleotide
ccttgaggtaccagagatct

SEQ ID NO: 56 - DNA, 20 bp, Artificial Sequence
  synthetic oligonucleotide
tgcctcaaattggactttgg

SEQ ID NO: 57 - DNA, 22 bp, Artificial Sequence
  synthetic oligonucleotide
gattgaaattctgtgtaactgc

SEQ ID NO: 58 - DNA, 20 bp, Artificial Sequence
  synthetic oligonucleotide
tgcctcaaattggactttgg

SEQ ID NO: 59 - DNA, 19 bp, Artificial Sequence
  synthetic oligonucleotide
cgctcgaggttaacgaatt

SEQ ID NO: 60 - DNA, 20 bp, Artificial Sequence
  synthetic oligonucleotide
gatgaactgaccaggcacta

SEQ ID NO: 61 - DNA, 20 bp, Artificial Sequence
  synthetic oligonucleotide
gtgggtcatatccactgtct

SEQ ID NO: 62 - DNA, 20 bp, Artificial Sequence
  synthetic oligonucleotide
gatgaactgaccaggcacta

SEQ ID NO: 63 - DNA, 20 bp, Artificial Sequence
  synthetic oligonucleotide
ccttgaggtaccagagatct

SEQ ID NO: 64 - DNA, 20 bp, Artificial Sequence
  synthetic oligonucleotide
ccctaggggatgttccagat

SEQ ID NO: 65 - DNA, 20 bp, Artificial Sequence
  synthetic oligonucleotide
tgaagcttttccctcttcca

SEQ ID NO: 66 - DNA, 20 bp, Artificial Sequence
  synthetic oligonucleotide
agcttggtggtggatgaaac

SEQ ID NO: 67 - DNA, 20 bp, Artificial Sequence
  synthetic oligonucleotide
ccctcttcagcaaagcagac

SEQ ID NO: 68 - DNA, 20 bp, Artificial Sequence
  synthetic oligonucleotide
ctagaccgtgggttttgcat

SEQ ID NO: 69 - DNA, 20 bp, Artificial Sequence
  synthetic oligonucleotide
tgggttaagtgcccctgtag

SEQ ID NO: 70 - DNA, 20 bp, Artificial Sequence
  synthetic oligonucleotide
acccagttcatagcggtgac

SEQ ID NO: 71 - DNA, 20 bp, Artificial Sequence
  synthetic oligonucleotide
caattgtcatgggattgcag

SEQ ID NO: 72 - DNA, 26 bp, Artificial Sequence
  synthetic oligonucleotide
atggaaactctattaaagtgaacctg

SEQ ID NO: 73 - DNA, 25 bp, Artificial Sequence
  synthetic oligonucleotide
tagacctcatactcagcattccagt

SEQ ID NO: 74 - DNA, 20 bp, Artificial Sequence
  synthetic oligonucleotide
gcgttggaacagaggttgga

SEQ ID NO: 75 - DNA, 20 bp, Artificial Sequence
  synthetic oligonucleotide
tgggagcaaagatccaagac

SEQ ID NO: 76 - DNA, 23 bp, Artificial Sequence
  synthetic oligonucleotide
gcaaatggtaaaggcaaatacgg

SEQ ID NO: 77 - DNA, 30 bp, Artificial Sequence
  synthetic oligonucleotide
aagaaaatatctgacgtttacaacatctaa

SEQ ID NO: 78 - DNA, 18 bp, Artificial Sequence
  synthetic oligonucleotide
aaagacctcgatgaagtt

SEQ ID NO: 79 - DNA, 18 bp, Artificial Sequence
  synthetic oligonucleotide
aggttctgcttttacctg

SEQ ID NO: 80 - DNA, 19 bp, Artificial Sequence
  synthetic oligonucleotide
cggctgaggaagctgagga

SEQ ID NO: 81 - DNA, 18 bp, Artificial Sequence
  synthetic oligonucleotide
aggttctgcttttacctg

SEQ ID NO: 82 - DNA, 18 bp, Artificial Sequence
  synthetic oligonucleotide
catgactaggatggttca

SEQ ID NO: 83 - DNA, 18 bp, Artificial Sequence
  synthetic oligonucleotide
cctgttataaagggcctg

SEQ ID NO: 84 - DNA, 18 bp, Artificial Sequence
  synthetic oligonucleotide
catgactaggatggttca

SEQ ID NO: 85 - DNA, 22 bp, Artificial Sequence
  synthetic oligonucleotide
tgtgtgcaaggtccaggatcag

SEQ ID NO: 86 - DNA, 20 bp, Artificial Sequence
  synthetic oligonucleotide
accacctagaggggaaagtg

SEQ ID NO: 87 - DNA, 18 bp, Artificial Sequence
  synthetic oligonucleotide
tagctactaaggaatgtg

SEQ ID NO: 88 - DNA, 34 bp, Artificial Sequence
  synthetic oligonucleotide
ataacttcgtataatgtatgctatacgaagttat

(19) United States
(12) Patent Application Publication
(10) Pub. No.: US 2011/0151447 A1
(43) Pub. Date: Jun. 23, 2011

(54) METHOD TO PRODUCE INDUCED PLURIPOTENT STEM (IPS) CELLS FROM NON-EMBRYONIC HUMAN CELLS

(75) Inventors: In-Hyun Park, Boston, MA (US); George Q. Daley, Weston, MA (US); Suneet Agarwal, Belmont, MA (US); Paul Hubert Lerou, Newton, MA (US)

(73) Assignee: CHILDREN'S MEDICAL CENTER CORPORATION, BOSTON, MA (US)

(21) Appl. No.: 12/741,632

(22) PCT Filed: Nov. 6, 2008
(86) PCT No.: PCT/US08/12532

§ 371 Date: Mar. 9, 2011

Related U.S. Application Data
(60) Provisional application No. 61/002,026, filed on Nov. 6, 2007.
(60) Provisional application No. 61/069,525, filed on Mar. 14, 2008.
(60) Provisional application No. 61/137,491, filed on Jul. 31, 2008.

(51) Int. Cl. C12N 5/071; C12Q 1/68; C12N 15/85; C12N 5/10

(52) U.S. Cl. 435/6.1; 435/366; 435/455

(57) ABSTRACT

The invention provides methods for generating induced pluripotent stem (iPS) cells from normal and mutant adult cells, as well as the iPS cells so generated from such methods. In some aspects, iPS cells are generated by ectopically expressing SOX2 and OCT4 nucleic acids in such adult cells. Other nucleic acids such as but not limited to MYC may also be ectopically expressed in such adult cells in the methods described herein.

### RELATED APPLICATIONS

This application claims priority to U.S. Provisional Patent Application Ser. No. 61/002,026, filed Nov. 6, 2007, Ser. No. 61/069,525, filed Mar. 14, 2008, and Ser. No. 61/137,491, filed Jul. 31, 2008, all entitled “METHOD TO PRODUCE INDUCED PLURIPOTENT STEM (IPS) CELLS FROM NON-EMBRYONIC HUMAN CELLS”, the entire contents of all of which are incorporated by reference herein.

### FIELD OF THE INVENTION

The invention relates to human pluripotent stem cells, methods for generating such cells from differentiated human cells, and screening methods for identifying factors that modulate this process.

### BACKGROUND OF THE INVENTION

Pluripotency, the capacity to generate all tissues in the organism, is a property of embryo-derived stem cells and can be induced in somatic cells by nuclear transfer into oocytes, fusion with pluripotent cells, and in the case of male germ cells, by cell culture alone (Wakayama et al., 2001; Cowan et al., 2005; Kanatsu-Shinohara et al., 2004). Pluripotent stem cells have a variety of therapeutic applications involving lineage or tissue regeneration. In particular, pluripotent stem cells that are derived from and thus genetically identical to an individual could be used to generate cells and/or tissues that would likely not give rise to graft versus host disease nor host versus graft disease upon transplant into the individual. Recently, pluripotent stem cells have been generated from murine fibroblasts (Takahashi and Yamanaka, 2006; Wernig et al., 2007; Okita et al., 2007; Maherali et al., 2007), but to date there have been no reports of successful isolation of pluripotent stem cells from human somatic tissues. The ability to generate these cells from somatic tissues is extremely desirable given the availability and accessibility of such tissues, and the vast therapeutic applications. It is unknown whether the approaches used to generate pluripotent stem cells from mouse fibroblasts would yield pluripotent stem cells from differentiated human cells. Therefore there still exists a need for a method for generating pluripotent stem cells from differentiated human somatic cells.

### SUMMARY OF THE INVENTION

The invention is based in part on the unexpected discovery that differentiated human cells can be reprogrammed into more immature precursor cells including but not limited to induced pluripotent stem (iPS) cells. The invention is further premised in part on the unexpected finding that iPS may be generated from mature cells derived from subjects having a genetic condition from a select subset of genetic conditions. In other words, it was found according to the invention that iPS could be generated from subjects having certain select genetic conditions rather than any genetic condition. Which genetic conditions were permissive with respect to iPS generation (and thus dedifferentiation of adult cells into immature precursors) and which were not could not be predicted a priori. Instead, it was unexpectedly found that some genetic conditions that were expected to interfere with the dedifferentiation process (and thus not yield iPS) actually did allow for iPS generation. Conversely, genetic conditions that were not expected to interfere with the dedifferentiation process actually did, and iPS cells could not be generated harboring such mutations.

The invention represents in part the first demonstration that normal and select mutant differentiated human cells can be de-differentiated (or reprogrammed), thereby acquiring developmental and differentiative potential that the cells had apparently lost during development. The resultant iPS cells generated were either normal (apart from the genes introduced into such cells in order to induce the dedifferentiation process) or were mutant to the extent that they harbored the same genetic mutation(s) carried by the subject from which they derived.

The invention provides methods for generating human iPS cells from differentiated human cells, as well as the iPS cells themselves. These human iPS cells may be normal or mutant as discussed in greater detail herein. The invention further provides methods for identifying factors that promote the reprogramming of human differentiated cells towards more immature precursors.

Thus, in one aspect, the invention provides a method for producing human induced pluripotent stem cells comprising ectopically expressing a SOX2 nucleic acid and an OCT4 nucleic acid in a differentiated human cell, and then culturing the differentiated human cell under culture conditions and for a time sufficient to generate (and thus detect) a human induced pluripotent stem cell derived from the differentiated human cell.

In one embodiment, the method further comprises ectopically expressing a MYC nucleic acid in the differentiated human cell in combination with the SOX2 nucleic acid and the OCT4 nucleic acid. In another embodiment, the method further comprises ectopically expressing a KLF-4 nucleic acid in the differentiated human cell in combination with the SOX2 nucleic acid and the OCT4 nucleic acid. In yet another embodiment, the method further comprises ectopically expressing an hTERT (i.e., human telomerase reverse transcriptase) nucleic acid (e.g., a nucleic acid encoding the catalytic subunit of human telomerase) in the differentiated human cell in combination with the SOX2 nucleic acid and the OCT4 nucleic acid. In still another embodiment, the method further comprises ectopically expressing an SV40 large T nucleic acid in the differentiated human cell in combination with the SOX2 nucleic acid and the OCT4 nucleic acid. In another embodiment, the method further comprises ectopically expressing a KLF-4 nucleic acid, a MYC nucleic acid, an hTERT nucleic acid, and an SV40 large T nucleic acid in the differentiated human cell in combination with the SOX2 nucleic acid and the OCT4 nucleic acid. In some embodiments, the culture conditions comprise the presence of a ROCK inhibitor.

In one embodiment, the differentiated human cell is a fibroblast, including but not limited to a fetal fibroblast and an adult fibroblast.

In one embodiment, the method further comprises harvesting the human induced pluripotent stem cells.

In one embodiment, the SOX2 nucleic acid is human SOX2 nucleic acid and the OCT4 nucleic acid is human OCT4 nucleic acid. In another embodiment, the SOX2 nucleic acid is mouse Sox2 nucleic acid and the OCT4 nucleic acid is mouse Oct4 nucleic acid.

In another aspect, the invention provides a composition comprising a population of human induced pluripotent stem cells produced according to any of the foregoing methods. In one embodiment, the population is a clonal population. The composition may comprise the population of human induced pluripotent stem cells in a pharmaceutically acceptable carrier. The carrier may be a liquid (e.g., sterile saline) or a solid or semi-solid (e.g., a hydrogel).

The invention in other aspects provides methods for producing human induced pluripotent stem cells from a subject having a genetic disease, disorder or condition.

In one aspect, the invention provides a method for producing human induced pluripotent stem cells from a subject having adenosine deaminase deficiency-related severe combined immunodeficiency (ADA-SCID) comprising ectopically expressing a SOX2 nucleic acid, an OCT4 nucleic acid and a KLF4 nucleic acid in a fibroblast obtained from the subject, and then culturing the fibroblast under culture conditions and for a time sufficient for detection of a human induced pluripotent stem cell derived from the fibroblast.

In one embodiment, the fibroblast is obtained from the subject when the subject is 1 year old or younger. In another embodiment, the fibroblast is obtained from the subject when the subject is 3 months of age.

In one aspect, the invention provides a method for producing human induced pluripotent stem cells from a subject having Gaucher disease comprising ectopically expressing a SOX2 nucleic acid, an OCT4 nucleic acid and a KLF4 nucleic acid in a fibroblast obtained from the subject, and then culturing the fibroblast under culture conditions and for a time sufficient for detection of a human induced pluripotent stem cell derived from the fibroblast.

In one aspect, the invention provides a method for producing human induced pluripotent stem cells from a subject having Duchenne type muscular dystrophy comprising ectopically expressing a SOX2 nucleic acid, an OCT4 nucleic acid and a KLF4 nucleic acid in a fibroblast obtained from the subject, and then culturing the fibroblast under culture conditions and for a time sufficient for detection of a human induced pluripotent stem cell derived from the fibroblast.

In one aspect, the invention provides a method for producing human induced pluripotent stem cells from a subject having Becker type muscular dystrophy comprising ectopically expressing a SOX2 nucleic acid, an OCT4 nucleic acid and a KLF4 nucleic acid in a fibroblast obtained from the subject, and then culturing the fibroblast under culture conditions and for a time sufficient for detection of a human induced pluripotent stem cell derived from the fibroblast.

In one aspect, the invention provides a method for producing human induced pluripotent stem cells from a subject having Down syndrome comprising ectopically expressing a SOX2 nucleic acid, an OCT4 nucleic acid and a KLF4 nucleic acid in a fibroblast obtained from the subject, and then culturing the fibroblast under culture conditions and for a time sufficient for detection of a human induced pluripotent stem cell derived from the fibroblast.

In one embodiment, the fibroblast is a foreskin fibroblast. In one embodiment, the fibroblast is a dermal fibroblast.

In one aspect, the invention provides a method for producing human induced pluripotent stem cells from a subject having Huntington disease comprising ectopically expressing a SOX2 nucleic acid, an OCT4 nucleic acid and a KLF4 nucleic acid in a fibroblast obtained from the subject, and then culturing the fibroblast under culture conditions and for a time sufficient for detection of a human induced pluripotent stem cell derived from the fibroblast.

In one aspect, the invention provides a method for producing human induced pluripotent stem cells from a subject having Pearson syndrome comprising ectopically expressing a SOX2 nucleic acid, an OCT4 nucleic acid and a KLF4 nucleic acid in a fibroblast obtained from the subject, and then culturing the fibroblast under culture conditions and for a time sufficient for detection of a human induced pluripotent stem cell derived from the fibroblast.

In one aspect, the invention provides a method for producing human induced pluripotent stem cells from a subject having Kearns-Sayre syndrome comprising ectopically expressing a SOX2 nucleic acid, an OCT4 nucleic acid and a KLF4 nucleic acid in a fibroblast obtained from the subject, and then culturing the fibroblast under culture conditions and for a time sufficient for detection of a human induced pluripotent stem cell derived from the fibroblast.

In one aspect, the invention provides a method for producing human induced pluripotent stem cells from a subject having retinoblastoma comprising ectopically expressing a SOX2 nucleic acid, an OCT4 nucleic acid and a KLF4 nucleic acid in a fibroblast obtained from the subject, and then culturing the fibroblast under culture conditions and for a time sufficient for detection of a human induced pluripotent stem cell derived from the fibroblast.

In one aspect, the invention provides a method for producing human induced pluripotent stem cells from a subject having Dyskeratosis congenita comprising ectopically expressing a SOX2 nucleic acid, an OCT4 nucleic acid and a KLF4 nucleic acid in a fibroblast obtained from the subject, and then culturing the fibroblast under culture conditions and for a time sufficient for detection of a human induced pluripotent stem cell derived from the fibroblast.

In one aspect, the invention provides a method for producing human induced pluripotent stem cells from a subject having Parkinson disease comprising ectopically expressing a SOX2 nucleic acid, an OCT4 nucleic acid and a KLF4 nucleic acid in a fibroblast obtained from the subject, and then culturing the fibroblast under culture conditions and for a time sufficient for detection of a human induced pluripotent stem cell derived from the fibroblast.

In one aspect, the invention provides a method for producing human induced pluripotent stem cells from a subject having juvenile type I diabetes mellitus comprising ectopically expressing a SOX2 nucleic acid, an OCT4 nucleic acid and a KLF4 nucleic acid in a fibroblast obtained from the subject, and then culturing the fibroblast under culture conditions and for a time sufficient for detection of a human induced pluripotent stem cell derived from the fibroblast.

In one aspect, the invention provides a method for producing human induced pluripotent stem cells from a subject having Shwachman-Bodian-Diamond syndrome (SBDS) comprising ectopically expressing a SOX2 nucleic acid, an OCT4 nucleic acid and a KLF4 nucleic acid in a mesenchymal cell obtained from the subject, and then culturing the mesenchymal cell under culture conditions and for a time sufficient for detection of a human induced pluripotent stem cell derived from the mesenchymal cell.

In one embodiment, the mesenchymal cell is a bone marrow mesenchymal cell. Various embodiments further comprise harvesting the human induced pluripotent stem cells. Various embodiments comprise ectopically expressing a MYC nucleic acid in the fibroblast or the mesenchymal cell in combination with the SOX2 nucleic acid, the OCT4 nucleic acid, and the KLF4 nucleic acid.

In various embodiments, the SOX2 nucleic acid is human SOX2 nucleic acid, and/or the OCT4 nucleic acid is human OCT4 nucleic acid, and/or the KLF4 nucleic acid is human KLF4 nucleic acid. In other embodiments, the SOX2 nucleic acid is mouse Sox2 nucleic acid, and/or the OCT4 nucleic acid is mouse Oct4 nucleic acid, and/or the KLF4 nucleic acid is mouse Klf4 nucleic acid.

In some embodiments, the MYC nucleic acid is human MYC nucleic acid. In other embodiments, the MYC nucleic acid is mouse Myc nucleic acid.

Various embodiments further comprise ectopically expressing an hTERT nucleic acid and an SV40 large T nucleic acid in the fibroblast or mesenchymal cell in combination with the SOX2 nucleic acid, the OCT4 nucleic acid, and the KLF4 nucleic acid.

In addition to the various embodiments recited above, the aforementioned methods may be carried out using any of the culture conditions as provided by the invention, including for example the use of a ROCK inhibitor, or the use of a retroviral construct bearing one, two, three or more of the genes required to dedifferentiate differentiated cells such as fibroblasts or mesenchymal cells.

In still other aspects, the invention provides the induced pluripotent stem cells produced according to the methods of the invention and compositions comprising such cells. Examples of such compositions include frozen aliquots, cultures, and suspensions.

Thus, in one aspect, the invention provides a composition comprising a human induced pluripotent stem that comprises an ADA-SCID mutation. In one aspect, the invention provides a composition comprising a human induced pluripotent stem that comprises a Gaucher disease mutation. In one aspect, the invention provides a composition comprising a human induced pluripotent stem that comprises a Duchenne type muscular dystrophy mutation. In one aspect, the invention provides a composition comprising a human induced pluripotent stem that comprises a Becker type muscular dystrophy mutation. In one aspect, the invention provides a composition comprising a human induced pluripotent stem that comprises a Down syndrome mutation. In one aspect, the invention provides a composition comprising a human induced pluripotent stem that comprises a Huntington disease mutation. In one aspect, the invention provides a composition comprising a human induced pluripotent stem that comprises a Pearson syndrome mutation. In one aspect, the invention provides a composition comprising a human induced pluripotent stem that comprises a Kearns-Sayre syndrome mutation. In one aspect, the invention provides a composition comprising a human induced pluripotent stem that comprises a retinoblastoma mutation. In one aspect, the invention provides a composition comprising a human induced pluripotent stem that comprises a Dyskeratosis congenita mutation. In one aspect, the invention provides a composition comprising a human induced pluripotent stem that comprises a Shwachman-Bodian-Diamond syndrome mutation.

In various embodiments, the human induced pluripotent stem cell is a population of induced pluripotent stem cells. In various embodiments, the human induced pluripotent stem cell comprises a retroviral nucleic acid comprising a SOX2 nucleic acid, an OCT4 nucleic acid, or a KLF4 nucleic acid.

In still other aspects, the invention provides particular species of induced pluripotent stem cells that comprise genetic mutations associated with particular conditions and compositions comprising such cell species. Thus, in one aspect, the invention provides a composition comprising ADA-iPS2 cells. In one aspect, the invention provides a composition comprising ADA-iPS3 cells. In one aspect, the invention provides a composition comprising GF-iPS1 cells. In one aspect, the invention provides a composition comprising GF-iPS3 cells. In one aspect, the invention provides a composition comprising DMD-iPS1 cells. In one aspect, the invention provides a composition comprising DMD-iPS2 cells. In one aspect, the invention provides a composition comprising BMD-iPS1 cells. In one aspect, the invention provides a composition comprising BMD-iPS4 cells. In one aspect, the invention provides a composition comprising DS1-iPS4 cells. In one aspect, the invention provides a composition comprising DS2-iPS1 cells. In one aspect, the invention provides a composition comprising DS2-iPS10 cells. In one aspect, the invention provides a composition comprising DS2-iPS10 cells. In one aspect, the invention provides a composition comprising PD-iPS1 cells. In one aspect, the invention provides a composition comprising PD-iPS5 cells. In one aspect, the invention provides a composition comprising JDM-iPS2 cells. In one aspect, the invention provides a composition comprising JDM-iPS4 cells. In one aspect, the invention provides a composition comprising SBDS-iPS1 cells. In one aspect, the invention provides a composition comprising SBDS-iPS3 cells. In one aspect, the invention provides a composition comprising HD-iPS4 cells. In one aspect, the invention provides a composition comprising HD-iPS11 cells.

The invention further provides in various aspects in vitro and in vivo methods for differentiating the normal and mutant iPS cells generated according to the methods of the invention. The differentiation methods may be those directed to generating any and all cell lineages or the cell lineage(s) that are affected by a particular mutation, in the case of the mutant iPS cells. The mutant iPS cells can also be used in screening methods aimed at identifying candidate therapeutics for the treatment of particular genetic conditions. These therapeutics may be gene therapies or small molecule therapies, or some combination thereof.

In another aspect, the invention provides a method for identifying a factor that promotes production of human induced pluripotent stem cells from differentiated human cells comprising ectopically expressing a SOX2 nucleic acid and an OCT4 nucleic acid in differentiated human cells in the presence and absence of a candidate factor, culturing the differentiated human cells under culture conditions and for a time sufficient for detection of a human induced pluripotent stem cell derived from the differentiated human cell, and measuring and comparing yield of human induced pluripotent stem cells produced in the presence and absence of the candidate factor. A yield of human induced pluripotent stem cells produced in the presence of the candidate factor that is greater than the yield in the absence of the candidate factor indicates a factor that promotes production of human induced pluripotent stem cells from differentiated human cells.

In one embodiment, the method further comprises ectopically expressing MYC nucleic acid in the differentiated human cells in combination with the SOX2 nucleic acid and the OCT4 nucleic acid.

In one embodiment, the candidate factor is a small molecule library member. In another embodiment, the candidate factor is a peptide or protein.

In one embodiment, the candidate factor is ectopically expressed in the differentiated human cells at the same time as the SOX2 nucleic acid and the OCT4 nucleic acid. The differentiated human cells may be fibroblasts, including but not limited to fetal fibroblasts. In another embodiment, the differentiated human cells are fibroblasts derived from differentiation of a human embryonic stem cell line.

In one embodiment, the culture conditions comprise the presence of a ROCK inhibitor.

In one embodiment, the SOX2 nucleic acid is human SOX2 nucleic acid and the OCT4 nucleic acid is human OCT4 nucleic acid. In another embodiment, the SOX2 nucleic acid is mouse Sox2 nucleic acid and the OCT4 nucleic acid is mouse Oct4 nucleic acid.

In still another aspect, the invention provides a method for identifying a factor that promotes production of human induced pluripotent stem cells from differentiated human cells comprising ectopically expressing a OCT4 nucleic acid and a MYC nucleic acid in differentiated human cells in the presence and absence of a candidate factor, culturing the differentiated human cells under culture conditions and for a time sufficient for detection of a human induced pluripotent stem cell derived from the differentiated human cell, and measuring and comparing yield of human induced pluripotent stem cells produced in the presence and absence of the candidate factor. A yield of human induced pluripotent stem cells produced in the presence of the candidate factor that is greater than the yield in the absence of the candidate factor indicates a factor that promotes production of human induced pluripotent stem cells from differentiated human cells.

In one embodiment, the candidate factor is a small molecule library member. In another embodiment, the candidate factor is a peptide or protein.

In one embodiment, the candidate factor is ectopically expressed in the differentiated human cells in combination with the OCT4 nucleic acid and the MYC nucleic acid. The differentiated human cells may be fibroblasts, including but not limited to fetal fibroblasts. The differentiated human cells may be fibroblasts derived from differentiation of a human embryonic stem cell line.

In one embodiment, the culture conditions comprise the presence of a ROCK inhibitor.

In one embodiment, the SOX2 nucleic acid is human SOX2 nucleic acid and the OCT4 nucleic acid is human OCT4 nucleic acid. In another embodiment, the SOX2 nucleic acid is mouse Sox2 nucleic acid and the OCT4 nucleic acid is mouse Oct4 nucleic acid.

In another aspect, the invention provides a method for producing human induced pluripotent stem cells comprising introducing a polycistronic nucleic acid that comprises an OCT4 nucleic acid, a SOX2 nucleic acid, and a KLF4 nucleic acid into a differentiated human cell, ectopically expressing the OCT4, SOX2, and KLF4 nucleic acids in the differentiated human cell, and then culturing the differentiated human cell under culture conditions and for a time sufficient for detection of a human induced pluripotent stem cell derived from the differentiated human cell.

In another aspect, the invention provides a method for producing human induced pluripotent stem cells comprising introducing a polycistronic nucleic acid that comprises an OCT4 nucleic acid, a SOX2 nucleic acid, a KLF4 nucleic acid, and a MYC nucleic acid into a differentiated human cell, ectopically expressing the OCT4, SOX2, KLF4 and MYC nucleic acids in the differentiated human cell, and then culturing the differentiated human cell under culture conditions and for a time sufficient for detection of a human induced pluripotent stem cell derived from the differentiated human cell.

In another aspect, the invention provides a method for producing human induced pluripotent stem cells comprising introducing a polycistronic nucleic acid that comprises an OCT4 nucleic acid, a SOX2 nucleic acid, a NANOG nucleic acid, and a LIN28 nucleic acid into a differentiated human cell, ectopically expressing the OCT4, SOX2, NANOG and MYC nucleic acids in the differentiated human cell, and then culturing the differentiated human cell under culture conditions and for a time sufficient for detection of a human induced pluripotent stem cell derived from the differentiated human cell.

In some embodiments, the polycistronic nucleic acid further comprises 2A nucleic acids that encode amino acid sequences selected from the group consisting of SEQ ID NOs: 22, 23, 24 and 25. In some embodiments, the 2A nucleic acids are the F2A, E2A, T2A and/or P2A sequences comprised within SEQ ID NOs: 19, 20 and 21. In some embodiments, the polycistronic nucleic acid further comprises loxP sites. In some embodiments, the loxP site has a sequence identical to the sequence of the loxP site in pEYK3.1.

In some embodiments, the polycistronic nucleic acid has a nucleotide sequence of SEQ ID NO:19. In some embodiments, the polycistronic nucleic acid has a nucleotide sequence of SEQ ID NO:20. In some embodiments, the polycistronic nucleic acid has a nucleotide sequence of SEQ ID NO:21.

In some embodiments the OCT4, SOX2, KLF4, MYC, NANOG and LIN28 sequences are all human sequences, while in some other embodiments they are all murine sequences. In still some embodiments, some of the sequences are human while others are murine.

In some embodiments, the method further comprises removing the polycistronic nucleic acid from the human induced pluripotent stem cell or its progeny using a Cre recombinase.

In some embodiments, the differentiated human cell is a fibroblast such as but not limited to a fibroblast derived from differentiating H1 ES cells (e.g., a dH1f cell). In some embodiments, the differentiated human cell is a fetal fibroblast cell such as a fetal fibroblast cell from an ADA-SCID human subject. In some embodiments, the differentiated human cell is a fetal skin fibroblast such as but not limited to a Detroit 551 cell.

In still a further aspect, the invention provides an induced pluripotent stem cell generated according to any of the foregoing methods, wherein the cell comprises one or more polycistronic nucleic acids in its genome. In some embodiments, the cell comprises 2 or 3 polycistronic nucleic acids in its genome.

These and other embodiments of the invention will be described in greater detail herein.

Each of the limitations of the invention can encompass various embodiments of the invention. It is therefore anticipated that each of the limitations of the invention involving any one element or combinations of elements can be included in each aspect of the invention.

This invention is not limited in its application to the details of construction and/or the arrangement of components set forth in the following description or illustrated in the Figures. The invention is capable of other embodiments and of being practiced or of being carried out in various ways.

The phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF THE FIGURES

FIG. 1. Change in morphology and gene expression during the differentiation of H1.1 human ES cells expressing GFP and Neomycin resistant gene in OCT4 locus (H1.1OGN). After differentiation for 4 weeks, differentiated H1.1OGN (dH1.1 fs) show fibroblast-like morphology (A) and lose the expression of GFP from OCT4 locus (B). The expression of pluripotency genes (OCT4, SOX2, and NANOG) is completely lost after 4 weeks of differentiation (C). (A) shows light phase pictures of H1.1OGN during differentiation. (B) is a FACS analysis of GFP before differentiation (H1.1OGN) and after 4 weeks of differentiation (dH1.1 fs). hFib2 was used as negative control for GFP expression. (C) shows data from a quantitative RT-PCR performed with RNA samples during H1.1OGN differentiation for the expression of OCT4, SOX2, NANOG, MYC and KLF4.

FIG. 2. Isolation of hiPS cells and expression of hES cell specific markers. hiPS cell line (A) expresses alkaline phosphatase (B), OCT4 (C) and (D), SSEA3 (E) and (F), SSEA4 (G) and (H), TRA-1-60 (I) and (J), and TRA-1-81 (K) and (L). (C), (E), (G), (I), and (K) were stained for antibody against the indicated antigen, and (D), (F), (H), (J), and (L) were stained with DAPI for the same cells.

FIG. 3. Colony of hiPS cells from adult dermal fibroblast cells (hFib2) 9 days after infection with OCT4, SOX2, KLF4, MYC, hTERT and SV40 Large T expressing retrovirus.

FIG. 4. Differentiation of human embryonic fibroblasts from human embryonic stem cells (H1-OGN). In the human ES cell line H1-OGN (Zwaka and Thomson, 2003), the OCT4 promoter drives expression of GFP-IRES-neo. (A) Time course of differentiation of H1-OGN cells into a population of adherent fibroblasts, and subsequent expansion of a colony into a clonal fibroblast cell line (dH1cf32). The differentiated fibroblast derivatives of H1-OGN cells are morphologically indistinguishable from dermal fibroblasts cultured from an adult volunteer donor (hFib2). (B) Quantitative real-time PCR demonstrates that the expression of a cohort of key pluripotency factors (OCT4, SOX2, NANOG and KLF4) is lost by the third week of differentiation, whereas expression of a fifth factor (MYC) persists.

FIG. 5. Multiple cultured human primary somatic cells yield iPS cells. (A) iPS cells produced from five independent human primary cell lines form colonies with a similarly compact, ES-cell-like morphology in co-culture with mouse embryonic feeder fibroblasts (MEFs). (B)-(F) As shown via immunohistochemistry (IHC), human iPS cell colonies express markers common to pluripotent cells, including alkaline phosphatase (AP), Tra-1-81, NANOG, OCT4, Tra-1-60, SSEA3 and SSEA4. 4,6-Diamidino-2-phenylindole (DAPI) staining indicates the total cell content per field. Fibroblasts surrounding human iPS colonies serve as internal negative controls for IHC staining. dH1f-iPS3-3 (B, from H1-OGN differentiated fibroblasts), MRC5-iPS2 (C, from MRC5 human fetal lung fibroblasts), BJ1-iPS1 (D, from neonatal foreskin fibroblasts), MSC-iPS1 (E, from mesenchymal stem cells), hFib2-iPS2 (F, dermal fibroblast from healthy adult male).

FIG. 6. Gene expression in human iPS cells is similar to human ES cells. (A-E) Quantitative realtime PCR assay for expression of OCT4, SOX2, NANOG, MYC, KLF4, hTERT, REX1 and GDF3 in human iPS and parental cells. Individual PCR reactions were normalized against internal controls (β-actin) and plotted relative to the expression level in the parent fibroblast cell line. (A) dH1f, dH1f-iPS3-3, dH1cf16-iPS-1 and dH1cf32-iPS-2 cells. (B) MRC5-iPS2, MRC5-iPS12 and MRC5-iPS17. (C) BJ1-iPS1. (D) MSC-iPS1. (E) hFib2-iPS2 and hFib2-iPS4. (F) Transgene-specific PCR primers permit determination of the relative expression levels between total, endogenous (Endo) and retrovirally expressed (Transgene) genes (OCT4, SOX2, MYC and KLF4) via semi-quantitative PCR. β-Actin is shown as a positive amplification and loading control.

FIG. 7. iPS cells are demethylated at the OCT4 and NANOG promoters relative to their fibroblast parent lines. Bisulphite sequencing analysis of the OCT4 and NANOG promoters in H1-OGN human ES cells, dH1f differentiated fibroblasts, dH1f-iPS-1, dH1cf32-iPS2, as well as the MRC5 neonatal foreskin fibroblast line and its derivatives MRC5-iPS2 and MRC5-iPS19. Each horizontal row of circles represents an individual sequencing reaction for a given amplicon. White circles represent unmethylated CpG dinucleotides; black circles represent methylated CpG dinucleotides. The cell line is indicated to the left of each cluster. The values above each column indicate the CpG position analysed relative to the downstream transcriptional start site (TSS). The percentage of all CpGs methylated (% Me) for each promoter per cell line is noted to the right of each panel.

FIG. 8. Global gene expression analysis of iPS cells. (A) A Pearson correlation was calculated and hierarchical clustering was performed with the average linkage method in H1-OGN, dH1f, dH1fiPS3-3, dH1cf16, dH1cf-iPS cells (dH1cf16-iPS5 and dH1cf32-iPS2), MRC5, MRC5-iPS2, BJ1 and BJ1-iPS1 cells. The distance metric calculated by GeneSpring GX7.3.1 for comparisons between different cell lines is indicated above the tree lines. The fibroblast lines dH1f, dH1cf16, MRC5 and BJ1 cluster together, whereas iPS cells cluster together with the H1-OGN human ES cell line. (B) Global gene expression patterns were compared between differentiated fibroblasts (dH1f, dH1cf16), reprogrammed somatic cells (dH1f-iPS3-3, MRC5-iPS2) and human ES cells (H1-OGN). Red lines indicate the linear equivalent and twofold changes in gene expression levels between the paired samples.

FIG. 9. Xenografts of human iPS cells generate well-differentiated teratoma-like masses containing all three embryonic germ layers. Immunodeficient mouse recipients were injected with human iPS cells (dH1f-iPS3-3) intramuscularly. Resulting teratomas demonstrate the following features in ectoderm, mesoderm and endoderm. Ectoderm: pigmented retinal epithelium (A), neural rosettes (B), glycogenated squamous epithelium (C); mesoderm: muscle (D), cartilage (E), bone (F); endoderm: respiratory epithelium (G). Of note, panel c contains all three germ layers: (1) glycogenated squamous epithelium, (2) immature cartilage, (3a) glandular tissue with surrounding stromal elements, and (3b) another small gland. All images were obtained from the same tumour. Tissue sections were stained with haematoxylin and eosin. Scale bar, 100 mm.

FIG. 10. Differentiation of hES cells results in transcriptional inactivity at the OCT4 locus. The H1-OGN hES cell line expresses GFP-ires-neo under the control of an endogenous OCT4 promoter, as demonstrated via flow cytometry where GFP positivity (45.7%) is apparent in undifferentiated cultures. Differentiation of H1-OGN to fibroblasts (dH1f) results in the loss of OCT4 expression as shown by the loss of GFP signal (0.26%).

FIG. 11. Viral integration site analysis indicates parent fibroblast lines and their derivative iPS cells have a common origin from single cell clones. Parent fibroblast lines were virally-infected with constructs encoding the fluorescent protein dTomato. Digestion of genomic DNA to reveal unique lentiviral integration sites from parent fibroblast lines and their corresponding iPS cell progeny, Southern blotting, and probing against the dTomato locus indicates common fragments, supporting a common, clonal origin for the iPS cell lines. dH1cf16 is the clonal, parent fibroblast line to dH1cf16-iPS1 and 5; dH1cf32 is the clonal, parent fibroblast line to dH1cf32-iPS2 and 4; dH1cf34, which carries two lentiviral integrants of equal band intensity is the clonal, parent fibroblast line to dH1cf34-iPS1 and 2.

FIG. 12. Gene expression profile of pluripotency factors in parental fibroblast lines differs extensively from hES cells. Quantitative RT-PCR was used to evaluate the expression profiles at key pluripotency-associated genes (OCT4, SOX2, MYC, KLF4, and NANOG) in hES cells (H1-OGN), and a panel of fibroblasts: dH1f (H1-OGN derived fibroblasts), clonal dH1cf16, MRC5 human fetal lung fibroblasts, BJ1 neonatal foreskin fibroblasts, hFib2 adult human dermal fibroblasts, and MSC mesenchymal stem cells. PCR reactions were normalized against beta-actin and plotted relative to the expression in hES cells (H1-OGN). OCT4, SOX2, and NANOG were not expressed in any of the fibroblast lines tested. All fibroblasts indicated varying degrees of expression for both MYC and KLF4.

FIG. 13. DNA fingerprinting analysis confirms that iPS cell lines are derived from their parent lines and not contaminating hES cell lines. Primer sets known to detect a high degree of heterozygosity were employed in genomic DNA PCR reactions. Each primer pair spans a genomic region containing a highly variable number of tandem tetranucleotide repeats. The resulting amplification patterns qualitatively verify that each iPS line is derivative of its indicated parent line as follows (from left to right): the hES cell line H1-OGN was used to generate the differentiated fibroblast line dH1f; dH1f is the parent line to the iPS cell lines dH1f-iPS3-3 and -1, and the clonal lines dH1cf16-iPS5 and 2; MRC5 fetal lung fibroblasts are the parent line to the clonal lines MRC5-iPS2 and 19; BJ1 neonatal foreskin fibroblasts are the parent line to the clonal lines BJ1-iPS1 and 2; MSC mesenchymal stem cells are the parent line to the clonal line MSC-iPS1; hFib2 adult dermal fibroblasts are the parent line to the clonal lines hFib2-iPS2 and 3; BG01 is a normal, undifferentiated hES cell line and 293T is a human embryonic kidney cell line. PCR primer sets (top to bottom): D10S1214, D17S1290, D7S796, and D21S2055.

FIG. 14. Southern hybridization analysis reveals multiple integrations of the (A) OCT4 and (B) SOX2 transgenes. The parent hES cell (H1-OGN) shares bands in common with all derivative iPS cell lines, which reflect the endogenous loci for OCT4 and SOX2. Retrovirally-inserted transgenic copies of these genes are indicated by the various fragments of unique mobility in all iPS derivatives.

FIG. 15. Xenograft of human iPS cells derived from clonal embryonic fibroblast derivative of H1-OGN cells demonstrates well-differentiated teratoma-like mass containing all three embryonic germ layers. Immunodeficient mouse recipients were injected with dH1cf16-iPS-1 intramuscularly. Resulting teratomas demonstrate: Mesoderm—(A) bone; Endoderm—(B) respiratory epithelium, and Ectoderm—(C) pigmented retinal epithelium and (D) immature mesenchyme and neurectoderm. All images were obtained from the same tumor. Tissue sections were stained with haematoxylin and eosin. Scale bar=100 μm.

FIG. 16. In vitro differentiated human iPS cells demonstrate gene expression from all three embryonic germ layers. Semi-quantitative RT-PCR was performed on sections of undifferentiated (Undiff.) iPS cell cultures and cognate differentiated (Diff.) regions from within the same culture dish. Beta-actin is shown as a positive amplification and loading control. The iPS lines dH1cf32-iPS2 (from fibroblast differentiated H1-OGN hES cells), MRC5-iPS3 (from MRC5 fetal lung fibroblasts), and MSC-iPS1 (from mesenchymal stem cells) all demonstrate upregulation of characteristic, tissue-specific markers upon differentiation relative to their iPS cell controls including: Endoderm—GATA4 and alphafeto-protein (AFP), Mesoderm—RUNX1 and Brachyury, and Ectoderm—NESTIN and N-CAM.

FIG. 17. Hematopoietic colony-forming assays demonstrate blood cell formation from human iPS cells. When differentiated as embryoid bodies prior to plating into hematopoietic growth factor-containing methylcellulose media, human iPS cells form multiple types of hematopoietic cells including burst-forming unit erythroid (BFU-E) colonies as shown here. Scale bar=100 microns.

FIG. 18. Human fibroblast-derived iPS cells maintain a normal karyotype. High-resolution, G-banded karyotypes indicate a normal, diploid, male chromosomal content. Human iPS cells were passaged five times prior to karyotype analysis.

FIG. 19. Genotypic analysis of disease-specific iPS cell lines. (A) Two different, primary fibroblast specimens, DS1 and DS2 from male patients with Down syndrome (trisomy 21) were used to derive DS1-iPS4 and DS2-iPS10. Each has a 47, XY+21 karyotype over several passages (G-banding analysis). (B) Fibroblast (ADA and GBA) or bone marrow mesenchymal cells (SBDS) were used to generate iPS lines. Mutated alleles identical to the original specimens were verified by DNA sequencing. Adenosine deaminase deficiency line ADA-iPS2, a compound heterozygote: GGG to GAA double transition in exon 7 of one allele (G216R substitution); the second allele is an exon 10 frame-shift deletion (-GAAGA) (Hirschhorn et al., 1993). Shwachman-Bodian-Diamond syndrome line SBDS-iPS8 is also a compound heterozygote: point mutations at the IV2+2T>C intron 2 splice donor site and an IVS3-1G>A mutation of the SBDS gene (Austin et al., 2005). GD-iPS3 (Gaucher disease type III); a 1226A>G point mutation (N370S substitution) and a guanine insertion at nucleotide 84 of the cDNA (84GG) (Beutler et al., 1991). (C) Fibroblasts from patients diagnosed with either Duchenne (DMD) or Becker type muscular dystrophy (BMD): DMD-iPS1 has a deletion over exons 45-52 (multiplex PCR for the dystrophin gene). We could not determine a deletion in BMD-iPS1 using two different multiplex PCR sets though these assays do not cover the entire coding region. DMD2 is a patient control (exon 4 deletion). The control is genomic DNA from a healthy volunteer. Huntington disease (HD) is caused by a tri-nucleotide repeat expansion within the huntington locus. DNA sequencing shows that HD-iPS has one normal (<35 repeats) and one expanded allele (72 repeats). HD2 is a positive control from a second Huntington patient with one normal and one expanded allele (54 repeats). The control is genomic DNA from a healthy volunteer.

FIG. 20. Patient-derived iPS lines exhibit markers of pluripotency. ADA-iPS2, GD-iPS1, DMD-iPS1, BMD-iPS1, DS1-iPS4, DS2-iPS10, PD-iPS1, JDM-iPS1, SBDS-iPS1, HD-iPS4, JDM-iPS2 were established from fibroblast or mesenchymal cells (Table 3). Disease specific iPS cell lines maintain a morphology similar to hES cells when grown in co-culture with mouse embryonic feeder fibroblasts (MEFs). Patient-specific iPS cells express alkaline phosphatase (AP). Also, as shown here via immunohistochemistry, patient-specific cells express pluripotency markers including Tra-1-81, NANOG, OCT4, Tra-1-60, SSEA3 and SSEA4. 4,6-Diamidino-2-phenylindole (DAPI) staining is shown at right and indicates the total cell content per image.

FIG. 21. Expression of pluripotency-associate genes is elevated in patient-specific iPS lines relative to their somatic cell controls. In each panel, quantitative real-time PCR (QRT-PCR) assays for OCT4, SOX2, NANOG, REX1, GDF3, and hTERT indicates increased expression in patient-specific iPS cells relative to parent cell lines while expression of KLF4 and cMYC remains largely unchanged. PCR reactions were normalized against internal controls (β-actin) and plotted relative to expression levels in their individual parent fibroblast cell lines. (A) Human iPS lines ADA-iPS2 and -iPS3 are derived from the adenosine deaminase deficiency-severe combined immunodeficiency fibroblast line ADA. (B) GD-iPS1 and -iPS3 are derived from the Gaucher disease type III fibroblast line GD. (C) DMD-iPS1 and -iPS2 are derived from the Duchenne muscular dystrophy fibroblast line DMD. (D) BMD-iPS1 and -iPS4 are derived from the Becker muscular dystrophy line BMD. (E) DS1-iPS4 is derived from the Down syndrome fibroblast line DS1. (F) DS2-iPS1 and -iPS10 are derived from the Down syndrome fibroblast line DS2. (G) PD-iPS1 and -iPS5 are derived from the Parkinson disease fibroblast line PD. (H) JDM-iPS2 and -iPS4 are derived from the juvenile-onset, type 1 diabetes mellitus line JDM. (I) SBDS-iPS1 and -iPS3 are derived from the Shwachman-Bodian-Diamond syndrome bone marrow mesenchymal fibroblast line SBDS. (J) HD-iPS4 and -iPS11 are derived from the Huntington disease fibroblast line HD. (K) Detroit 551 human fibroblasts are used as the standard here in order to demonstrate the previously described expression pattern in Detroit 551 derived iPS cells (551-iPS8) relative to two bona fide hES cell lines: H1-OGN and BG01.

FIG. 22. Pluripotency-promoting genes are chiefly expressed from the endogenous loci in patient-specific iPS lines, while the virally-delivered transgene is predominantly silenced. The patient-specific iPS cell lines shown here are preceded by their parental fibroblast controls (from left to right at top): adenosine deaminase deficiency-associate severe combined immunodeficiency (ADA), Becker muscular dystrophy (BMD), Parkinson disease (PD), juvenile type one diabetes mellitus (JDM), Huntington disease (HD), Detroit 551 control cells, Duchenne muscular dystrophy (DMD), Shwachman-Bodian-Diamond syndrome (SBDS), Down syndrome (DS), and Gaucher disease type III (GD). The semi-quantitative expression (RT-PCR) of the four pluripotency-promoting genes used in the reprogramming process, OCT4, SOX2, cMYC, and KLF4 is shown for each line using amplification conditions specific to the endogenous (Endo) or virally-delivered transgene (Trans) as well as the total expression for each (Total). Beta-actin is shown at the bottom as a loading control for each lane.

FIG. 23. Differentiation of patient-specific iPS lines reveals lineage-specific gene expression and mature cell formation. (A) At top (from left to right) are nine iPS cell lines in their undifferentiated (U) or differentiated (D) state. The lines are adenosine deaminase deficiency-associated severe combined immunodeficiency (ADA), juvenile-onset type one diabetes mellitus (JDM), Down syndrome 1 (DS1), Gaucher disease type III (GD), Huntington disease (HD), Duchenne muscular dystrophy (DMD), Down syndrome 2 (DS2), and normal control Detroit 551 (551) cells. Differentiation (D) of these patient-specific iPS cells as embryoid bodies (EB) followed by RT-PCR analysis shows upregulated expression of lineage markers from the three embryonic germ layers relative to their undifferentiated controls (U) including GATA4 and AFP (endoderm), RUNX1 and Brachyury (mesoderm), and Nestin and NCAM (ectoderm). Beta-actin serves as a positive amplification control for each. (B) Differentiation of ADA-iPS2, a representative patient-specific iPS cell line, as embryoid bodies (EB) is highly reminiscent of that using hES cells where tight clusters of differentiating cells are well-formed by day 7 which will cavitate, becoming cystic, by day 10. Hematopoietic differentiation of patient-specific iPS cells yields various blood cell types in semi-solid methylcellulose colony-forming assays including burst-forming unit-erythroid (BFU-E) which are derivative of red blood cell progenitor cells.

FIG. 24. Patient-specific iPS lines form teratomas in immunodeficient mice. Shown here are the representative series of hematoxylin-eosin (H/E) stained sections from a formalin fixed teratoma produced from ADA-iPS2, BMD-iPS1, DS1-iPS4, HD-iPS1, PD-iPS1, SBDS-iPS3, and JDM-iPS1 cell lines. They formed mature, cystic teratomas with tissues representing all three embryonic germ layers including: respiratory epithelium (endoderm), bone and cartilage (mesoderm), and pigmented retinal epithelium and immature neural tissue (ectoderm).

FIG. 25. Qualitative DNA fingerprint analysis indicates that each line is derivative of its indicated parental fibroblast source. PCR-based DNA fingerprint analysis using primer sets spanning highly variable tetra-nucleotide repeats are shown for four different loci: D7S796, repeat (GATA)n, average heterozygosity 0.95; D21S2055, repeat (GATA)n, average heterozygosity 0.88; D17S1290, repeat (GATA)n, average heterozygosity 0.84; and D10S1214, repeat (GGAA)n, average heterozygosity 0.97. Of note, the Down syndrome derived iPS lines (DS1-iPS4 and DS2-iPS3) as well as their respective parent fibroblasts (DS1 and DS2) each show three alleles at D21S2055 in keeping with the observation that most cases of DS derive from errors occurring within meiosis I of female germ cell development, where the two maternal amplicons represent alleles from each maternal grandparent with the third allele originating from within the paternal genome. From left to right at top are six lines of previously described (Park et al., 2008) human iPS cells: MRC5-iPS2 is a normal iPS line from fetal lung fibroblasts, BJ1-iPS4 is a normal iPS line from neonatal foreskin fibroblasts, MSC-iPS2 is a normal iPS line from mesenchymal fibroblasts, hFib2-iPS2 is a normal iPS line from adult fibroblasts, and 551-iPS8 is a normal fibroblast iPS line. These are followed (from left to right) by patient-specific iPS lines as well as their parental fibroblast controls: DMD=Duchenne muscular dystrophy, SBDS=Shwachman-Bodian-Diamond syndrome, DS=Down syndrome, GD=Gaucher disease type III, ADA=adenosine deaminase deficiency-associated severe combined immunodeficiency, BMD=Becker type muscular dystrophy, PD=Parkinson disease, JDM=juvenile-onset type one diabetes mellitus, HD=Huntington disease, H1-OGN and BG01 are human embryo-derived hES cells, and 293T is an immortalized human embryonic kidney-derived cell line used in the creation of the viral supernatants for reprogramming.

FIG. 26. Patient-specific iPS lines maintain normal karyotypes. When chromosomal contents were analyzed with high resolution G-banding karyotypes, ADA-iPS3, GD-iPS1, DMD-iPS1, BMD-iPS4, PD-iPS5, JDM-iPS1, SBDS-iPS3, and HD-iPS1 indicated normal, diploid chromosomal contents.

FIG. 27 is a schematic of the pEYK3.1 vector showing the loxP site in the LTR (designated by an arrow) and the GFP sequence that is substituted with the polycistronic constructs provided herein.

FIG. 28 is a schematic of the organization of the reprogramming factors within the E3, E4 and E4L (and correspondingly the M3, M4 and M4L) constructs. It is to be understood that the constructs further contain a downstream LTR and loxP site (although not illustrated in the Figure). Viral 2A sequences F2A, T2A, and E2A, as described herein, are used to separate the coding sequences of the reprogramming factors.

FIG. 29A-B are photographs of iPS cell colonies produced using dH1f cells and the M4 and M4L constructs respectively.

FIG. 30A-E are photographs of iPS cell colonies generated using the E4 construct using a starting cell population of ADA cells (A and C), 551 cells (B and D), and dH1f cells (E).

FIG. 31A-B is each a compilation of photographs showing expression of pluripotency markers by immunostaining in dH1f cells infected with the M4L construct.

FIG. 32A-D are photographs of Western blots showing OCT4 (A), SOX2 (B), KLF4 (C) and MYC (D) expression in iPS cell clones generated using M3, M4, M4L, E3, E4 and E4L constructs. Negative control (NEG) and positive control (OCT4, SOX2, KLF4, and MYC) are also shown.

FIG. 33A is a schematic showing the integrated E4 (or M4) and E4L (or M4L) constructs, the EcoRI sites (E), the HindIII sites (H), and the OCT4 (0), SOX2 (S), KLF4 (K), MYC (M), NANOG (N), and LIN28 (L) sequences. The probe used in the Southern blot hybridizes between the 5′ LTR and the OCT4 sequence. The probe binds to fragments that are in about the 2 kb range, although the length of each fragment will depend upon its integration site and the nearest genomic EcoRI or HindIII site.

FIG. 33B is a photograph showing data from a Southern blot carried out on a number of iPS cell clones generated using the polycistronic vectors of the invention. Each lane corresponds to one clone and the number of bands in each lane corresponds to the number of times the construct has integrated into the genome of that clone (i.e., the number of integration events). As shown, the number of integration events varies from at least 2 to about 8 per clone.

It is to be understood that the Figures are not required to enable the claimed invention.

### BRIEF DESCRIPTION OF THE SEQUENCE LISTING

SEQ ID NO:1 is the nucleotide sequence for human OCT4, transcript variant 1 (GenBank Accession No. NM002701).

SEQ ID NO:2 is the nucleotide sequence for mouse Oct4 (GenBank Accession No. NM 013633).

SEQ ID NO:3 is the nucleotide sequence for human SOX2 (GenBank Accession No. NM 003106).

SEQ ID NO:4 is the nucleotide sequence for mouse Sox2 (GenBank Accession No. NM 011443).

SEQ ID NO:5 is the nucleotide sequence for human MYC (GenBank Accession No. V00568).

SEQ ID NO:6 is the nucleotide sequence for mouse Myc (GenBank Accession No. NM 010849).

SEQ ID NO:7 is the nucleotide sequence for human KLF-4 (GenBank Accession No. NM 004235).

SEQ ID NO:8 is the nucleotide sequence for mouse Klf-4 (GenBank Accession No. MMU20344).

SEQ ID NO:9 is the nucleotide sequence of the ACTB forward primer.

SEQ ID NO:10 is the nucleotide sequence of the ACTB reverse primer.

SEQ ID NO:11 is the nucleotide sequence of the OCT4 forward primer.

SEQ ID NO:12 is the nucleotide sequence of the OCT4 reverse primer.

SEQ ID NO:13 is the nucleotide sequence of the NANOG forward primer.

SEQ ID NO:14 is the nucleotide sequence of the NANOG reverse primer.

SEQ ID NO:15 is the nucleotide sequence of the XIST forward primer.

SEQ ID NO:16 is the nucleotide sequence of the XIST reverse primer.

SEQ ID NO:17 is the nucleotide sequence of human telomerase reverse transcriptase (hTERT), transcript variant 1 (GenBank Accession No. NM_{—}198253).

SEQ ID NO:18 is the nucleotide sequence of SV40 LT (acquired through Addgene).

SEQ ID NO:19 is the nucleotide sequence of the EcoRI-OCT4-FMDV2-SOX2-T2A-KLF4-XhoI sequence present in the E3 (or M3) constructs.

SEQ ID NO:20 is the nucleotide sequence of the EcoRI-OCT4-FMDV2-SOX2-T2A-KLF4-E2A-MYC-XhoI sequence present in the E4 (or M4) constructs.

SEQ ID NO:21 is the nucleotide sequence of the EcoRI-OCT4-FMDV2-SOX2-T2A-NANOG-E2A-LIN28-XhoI sequence present in the E4L (or M4L) constructs.

SEQ ID NO:22 is the amino acid sequence of the FMDV2 (or F2A) sequence.

SEQ ID NO:23 is the amino acid sequence of the T2A sequence.

SEQ ID NO:24 is the amino acid sequence of the E2A sequence.

SEQ ID NO:25 is the amino acid sequence of the P2A sequence.

SEQ ID NO:26 is the nucleotide sequence for human NANOG (Ensembl No. ENST00000229307).

SEQ ID NO:27 is the nucleotide sequence for mouse Nanog (GenBank Accession No. NM 028016).

SEQ ID NO:28 is the nucleotide sequence for human LIN28 (Ensembl No. ENST00000254231).

SEQ ID NO:29 is the nucleotide sequence for mouse Lin28 (GenBank Accession No. NM 145833).

Nucleotide sequences from GenBank or other commercial sources (e.g., Addgene) are provided in the accompanying Sequence Listing. Those of ordinary skill in the art can use these sequences or they can refer directly to GenBank for the nucleotide sequences of interest.

### DETAILED DESCRIPTION OF THE INVENTION

The invention is based in part on the surprising discovery that differentiated human cells can be reprogrammed into immature precursor cells. These immature precursor cells are referred to herein as human induced pluripotent stem (hiPS) cells. Reprogramming occurs as a result of induced expression of transcription factors associated with pluripotency that are not normally expressed in the differentiated cells.

It has been further found in accordance with the invention that iPS cells may be generated from differentiated human cells from subjects having select conditions. It has been found unexpectedly that iPS cells can be generated from some but not all tested differentiated cells known to carry genetic mutations attributed to select conditions. It was not known prior to the invention which of the differentiated “mutant” cells could be dedifferentiated and which could not. Rather, certain mutant cells which were expected to yield iPS cells did not, and some mutant cells where were expected not to yield iPS cells did. Interestingly, although many attempts were made, it was not possible to produce iPS cells from differentiated cells from subjects having Fanconi anemia even using the same conditions and reagents used to generate many of the other “mutant” iPS cells provided herein. Additionally, the ability to generate iPS cells from subjects having Dyskeratosis congenita was also unexpected given that the mutation results in shorter (than normal) telomere length and therefore limited proliferative activity. However, unexpectedly, iPS cells from such subjects were generated. The telomere lengths in these iPS cells had not increased as a result of dedifferentiation. This is in itself unexpected since reprogramming such as occurs in a dedifferentiation process has been thought to be associated with reactivation of telomerase activity, resulting in an increase in telomere length. This was not the case with iPS cells derived from Dyskeratosis congenital subjects. It was also unexpected that differentiated cells from subjects having Pearson syndrome could be dedifferentiated due to the mitochondrial defect that characterizes this disorder. It was further unexpected to obtain a trisomy 21 containing iPS cell line from a Down syndrome subject since these subjects can be mosaics with not all cells harboring the mutation.

The invention therefore provides iPS cells that individually harbor (or carry or comprise) the genetic mutation(s) associated with adenosine deaminase deficiency-related severe combined immunodeficiency (ADA-SCID), Down syndrome, Gaucher disease, Duchenne type muscular dystrophy, Becker type muscular dystrophy, Huntington disease (e.g., Huntington chorea), Pearson syndrome, Kearns-Sayre syndrome, retinoblastoma, Shwachman-Bodian-Diamond syndrome (SBDS), Dyskeratosis congenita, Parkinson's disease, and juvenile type I diabetes mellitus. These mutant iPS cells were derived from primary fibroblasts or mesenchymal cells that are available from cell depositories such as Coriell and ATCC. Importantly, the invention therefore demonstrates that iPS cells can be generated from primary cells of subjects having select conditions. This provides the possibility that a patient specific iPS based therapy may be available to such subjects in the future.

The invention further provides methods for generating iPS cells from normal or mutant human cells using genetic vectors that comprise coding sequences for more than one of the reprogramming factors. Such vectors, which are referred to as polycistronic vectors, may comprise coding sequence for two, three, four or more reprogramming factors. In some instances, they comprise coding sequences for all the reprogramming factors used. The reprogramming factors may be selected from OCT4, SOX2, KLF4, NANOG, MYC and LIN28. Nucleic acids comprising two or more reprogramming factors, and optionally LTR sequences, and further optionally loxP sequences, are referred to herein as polycistronic nucleic acids. Such nucleic acids are non-naturally occurring and preferably further comprise one or more viral 2A sequences such as but not limited to F2A, T2A, E2A and P2A, the nucleotide sequences of some of which are provided in SEQ ID NOs: 19, 20 and 21, or are otherwise known in the art. An example of a wild type loxP sequence that can be used in accordance with the invention is 5′ ATAACTTCGTATA ATGTATGC TATACGAAGTTAT 3′ (SEQ ID NO: 88).

This aspect of the invention additionally provides for the removal of retroviral sequences from infected cells, thereby reducing or eliminating the risk of tumor formation that is associated with retroviral use in vivo. One mechanism for removal of the retroviral sequences is the use of the Cre/lox recombination system which excises from the genome sequences present between loxP sites using Cre recombinase.

hiPS cells are defined, according to the invention, as immature cells that resemble human embryonic stem (hES) cells in a number of respects. Morphologically, iPS cells are small round translucent cells that preferably grow in vitro in colonies that are themselves characterized as tightly packed and sharp-edged. Genetically, iPS cells express markers of pluripotency such as OCT4 and NANOG, cell surface markers such as SSEA3, SSEA4, Tra-1-60, and Tra-1-80, and the intracellular enzyme alkaline phosphatase. Consistent with these expression profiles, the OCT4 and NANOG promoters in these cells are demethylated to a greater extent than in differentiated cells (e.g., fibroblasts). These cells have a normal karyotype. The X chromosome in these cells appears activated and XIST expression is undetectable by PCR. Their cell cycle profile can be characterized by a short G1 phase, similar to that of hES cells.

The invention provides methods for producing hiPS cells from differentiated cells. These methods generally involve inducing the expression of certain transcriptional factors. Gene expression induction can be carried out in a number of ways, and the invention is not limited in this regard. The Examples demonstrate ectopic expression of these factors following retroviral transfection. Briefly, populations of differentiated cells were infected with retroviral supernatants carrying OCT4 and SOX2, and either or both KLF4 and MYC in some cases, and OCT4, SOX2, NANOG and LIN28 in other cases. Following retroviral infection, the cells are plated in culture conditions conducive to the growth and proliferation of human immature cells such as hES cells. For example, the cells can be cultured in hES cell culture medium, whether in the presence or absence of mouse embryonic fibroblasts (MEF). Various hES cell culture media are available commercially. An exemplary hES cell culture medium is described in greater detail in the Examples.

It has been found according to the invention that the production of mutant iPS cells described herein could be accomplished using a three factor cocktail of SOX2, OCT4 and KLF4 if differentiated cells from younger subjects (e.g., preferably subjects younger than a year). If differentiated cells from older subjects (e.g., subjects who are 15+ years old) then the four factor cocktail of SOX2, OCT4, KLF4 and MYC are preferred. For still other starting cell populations, a factor cocktail of OCT4, SOX2, NANOG and LIN28 was used.

In a preferred embodiment, the culture conditions include a Rho-associated kinase (ROCK) inhibitor. As used herein, a ROCK inhibitor is an agent that inhibits Rho-associated kinase. The inhibitor can be nucleic acid or amino acid in nature, and in some important embodiments it is a chemical compound, whether organic or inorganic in nature. In another preferred embodiment, the ROCK inhibitor is (R)-(+)-trans-N-(4-Pyridyl)-4-(1-aminoethyl)-cyclohexanecarboxamide, 2HCl. This compound is described in U.S. Pat. No. 4,997,834 and published PCT application WO98/06433A1 to Mitsubishi Pharma Corp., and by Watanabe et al., 2007. This compound is commercially available from Calbiochem as Y27632. Another example of a ROCK inhibitor is (5-isoquinolinesulfonyl)homopiperazine, 2HCl (also known as Fasudil HA 1077, Dihydrochloride; CAS103745-39-7) which is also commercially available from Calbiochem as HA1077.

The cultures are performed for a time sufficient for growth and proliferation, and thus ultimately detection, of hiPS cells. This time can vary depending on particular starting cell population. One of ordinary skill in the art will be able to determine this time using routine experimentation.

The differentiated cells are induced to express particular factors, referred to herein as reprogramming factors. These factors are SOX2, OCT4 (also known as POU5F1, OCT3 and OTF3), and optionally MYC, KLF-4 (also known as EZF and GKLF), NANOG and LIN28. In one embodiment, SOX2, OCT4 and MYC are used. In one embodiment, OCT4, SOX2 and KLF4 are used. In another embodiment, SOX2, OCT4, KLF4 and MYC are used. In still another embodiment, OCT4, SOX2, NANOG and LIN28 are used. In still other embodiments, additional factors can be used with any of the foregoing combinations. Additional factors include but are not limited to hTERT and SV40 Large T antigen (SV40 LT). Thus, in yet another embodiment, the factor combination is OCT4, SOX2, MYC, KLF4, hTERT, and SV40 LT. Preferably, all genes within a combination are ectopically expressed at the same time, there being no time lag between expression of one or another of the factors. This can be achieved for example when using retroviral infection by simultaneously infecting the differentiated cells with retroviral particles expressing the factors. In one important embodiment, each particle encodes only one of the factors. In other embodiments, each particle encodes two, three or all four factors.

To this end, the invention provides polycistronic genetic vectors, such as genetically engineered retroviruses, that encode two or more of the factors used to reprogram cells into iPS cells. The vectors may contain two, three, four or more of the reprogramming factors, including all of the reprogramming factors used. These vectors take advantage of viral mechanisms for generating polycistronic nucleic acids. One such mechanism is the use of 2A sequences which are described in de Felipe et al., Gene Therapy, 6:198-208, 1999; de Felipe et al., Human Gene Therapy, 11:1921-1931, 2000; and Luke et al., Biologist, 53(4):190-194, 2006. Examples of suitable 2A sequences include those from foot-and-mouth disease virus (FMDV) (referred to herein as F2A), equine rhinitis A virus (referred to herein as E2A), Thosea asigna (insect) virus (referred to herein as T2A), and porcine teschovirus-1 (referred to herein as P2A). The amino acid 2A sequences are shown and compared below, with the ultimate P residue being part of the 2B sequence of these viruses.

| | |
|---|---|
| F2A VKQTLNFDL L KLA GDVE S NPG P | (SEQ ID NO: 22) |
| E2A QCTNYAL L KLA GDVE S NPG P | (SEQ ID NO: 23) |
| T2A EGRGS L LTC GDVE E NPG P | (SEQ ID NO: 24) |
| P2A ATNFSL L KQA GDVE E NPG P | (SEQ ID NO: 25) |

The invention contemplates the use of any combination of these sequences to generate polycistronic nucleic acids and vectors. Nucleic acid sequences that encode these amino acid sequences are comprised in SEQ ID NOs: 19, 20 and 21. These sequences are important because they facilitate the production of a single mRNA species from the E3, E4, E4L (or M counterpart constructs) but also lead to the production of independent protein products. Therefore the E3 construct can yield a single mRNA species and that mRNA can yield three separate protein products (i.e., OCT4, SOX2 and KLF4 proteins). The E4 construct can yield a single mRNA species and that mRNA can yield four separate protein products (i.e., OCT4, SOX2, KLF4 and MYC proteins). And the E4L construct can yield a single mRNA species and that mRNA can yield four separate protein products also (i.e., OCT4, SOX2, NANOG and LIN28 proteins).

Examples of polycistronic sequences that have been generated using a plurality of 2A sequences include the EcoRI-OCT4-FMDV2-SOX2-T2A-KLF4-XhoI sequence (referred to herein as E3, SEQ ID NO:19), EcoRI-OCT4-FMDV2-SOX2-T2A-KLF4-E2A-MYC-XhoI sequence (referred to herein as E4, SEQ ID NO:20), and EcoRI-OCT4-FMDV2-SOX2-T2A-NANOG-E2A-LIN28-XhoI sequence (referred to herein as E4L, SEQ ID NO:21). The generation of these constructs is described in greater detail in the Examples. The invention contemplates various orders of the reprogramming factors within a polycistron, but in some preferred embodiments the order is identical to that of the E3, E4 and E4L constructs. Similarly, the invention contemplates the use of a variety of 2A sequences, and order of these sequences may vary from construct to construct. However, in some preferred embodiments, the choice and order of 2A sequences is identical to that of the E3, E4 and E4L constructs.

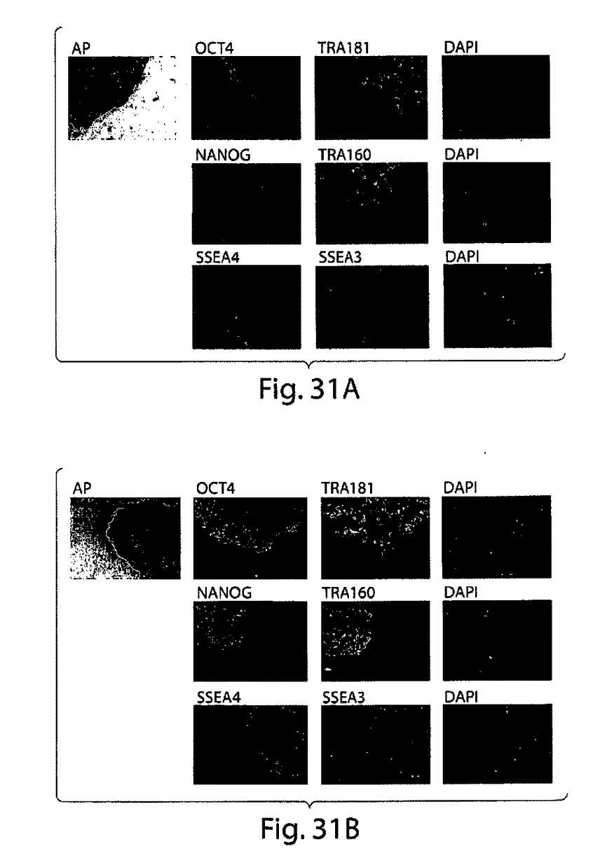

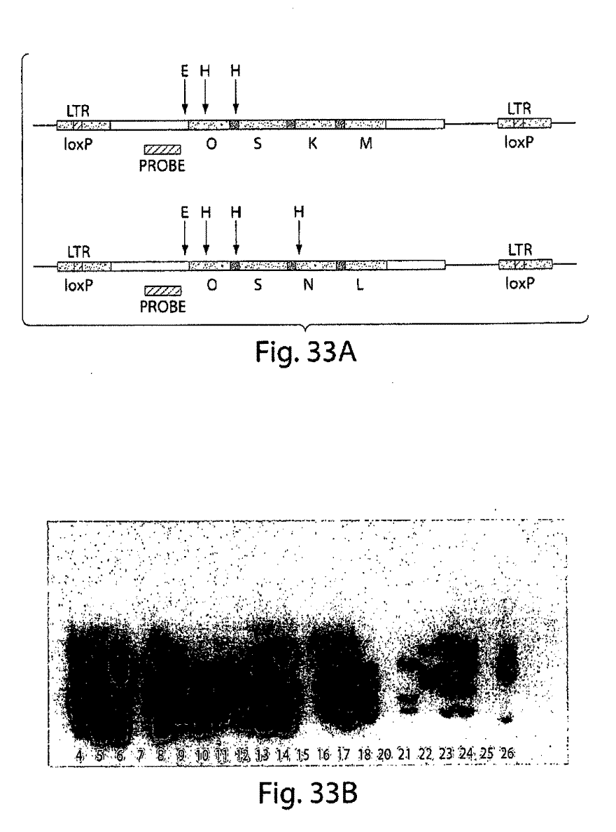

iPS cells have been generated from a number of starting cell populations using each of these polycistronic constructs. For example, 8 iPS cell clones have been generated using ADA cells (fetal fibroblasts from an ADA-SCID patient) as a starting population and the E3 construct, about 35 iPS cell clones have been generated using dH1f cells (differentiated H1-OGN fibroblasts) as a starting population and the E3 construct, 30 iPS cell clones have been generated using ADA cells as a starting population and the E4 construct, 12 iPS cell clones have been generated using dH1f cells as a starting population and the E4 construct, 20 iPS cell clones have been generated using 551 cells (Detroit 551 fetal skin fibroblasts) as a starting population and the E4 construct, 6 iPS cell clones have been generated using ADA cells as a starting population and the E4L construct, 48 iPS cell clones have been generated using dH1f cells as a starting population and the E4L construct, and 4 iPS cell clones have been generated using 551 cells as a starting population and the E4L construct. FIGS. 29 and 30 show representative iPS cell colonies generated using the OCT4-SOX2-KLF4-MYC construct and the OCT4-SOX2-NANOG-LIN28 construct from a variety of starting cell populations. These iPS cells contain anywhere from 2-8 integrations of the polycistron, as shown by Western analysis (FIG. 33B). They also express markers of pluripotency such as alkaline phosphatase, SSEA3, SSEA4, TRA-1-81 and TRA-1-60, as shown in FIGS. 31A and B.

The cDNA nucleotide sequences for the reprogramming factors are known and representative public database (such as GenBank) submissions are provided in the Sequence Listing. In some embodiments, the human sequences are used, while in others the mouse sequences are used. However, the invention contemplates use of a combination of human and mouse sequences. In one preferred embodiment, the human nucleotide sequences for OCT4, SOX2, KLF4 and MYC are used. In another embodiment, the mouse nucleotide sequences for Oct4, Sox2 and Klf4 and the human nucleotide sequence for MYC are used.

These factors are ectopically expressed in the starting differentiated cell or cell population. As used herein, ectopic expression refers to the expression of a gene (and its associated gene product) in a cell or cell population that doesn't normally express the gene or gene product. For example, OCT4 and SOX2 are “ectopically expressed” in differentiated fibroblasts, according to the invention, because differentiated fibroblasts do not normally express these genes (i.e., in the absence of any genetic manipulation of these cells, they would not express these genes). Ectopic expression can come about by any method and the invention is not so limited.

Exemplary protocols are described in the Examples. Briefly, this protocol involves infecting differentiated fibroblasts with OCT4, SOX2, MYC and KLF4 for 3-4 days, and then splitting the cell cultures and reculturing on mouse embryonic fibroblasts (MEF) for another 3-4 days. At this point, small colonies resembling hES cell colonies growing in contact with the MEF become apparent. The media is then changed to hES cell medium containing a ROCK inhibitor such as Y27632. The cultures are maintained for a total of about 14-15 days post infection, at which time colonies are picked, expanded and further characterized. As described herein, the cells are analyzed for cell surface expression of SSEA3, SSEA4, TRA-1-60 and TRA-1-80, protein expression of OCT4, and alkaline phosphatase enzyme activity.

The starting differentiated cell population can be a fibroblast population, although the invention is not so limited. In some embodiments, the fibroblast population is a fetal fibroblast population. The Examples demonstrate production of hiPS cells from the fetal fibroblast cell line MRC5 (ATCC Accession No. ATCC CCL-171). In some embodiments, the fibroblast population is an adult fibroblast population such as an adult dermal fibroblast. In other embodiments, it may be a related cell type such as but not limited to a mesenchymal stem cell.

The Examples further demonstrate production of hiPS cells from a population of differentiated fibroblasts derived from hES cells. This latter population is a cell line of fibroblasts differentiated from the H1.1OGN hES cell line. H1.1OGN is a derivative of the H1.1 hES cell line that includes the green fluorescent protein (GFP) gene and the neomycin resistance (neoR) gene under the control of the OCT4 promoter. As demonstrated in the Examples, these cells can be used to select for or against the presence of hES cells in a population based on one or both of these selectable markers. For example, as shown in the Examples, cells differentiated from H1.1OGN hES cells can be subjected to G418 in order to determine whether any hES cells are still in the population since only those cells will be resistant to the drug selection. GFP can be used in a similar manner except that the non-fluorescent differentiated cells would still be viable.

A differentiated fibroblast cell line has been generated from the H1.1OGN line. The line was derived as follows: The H1.1OGN cell line was cultured to form ES cell colonies, at which point the cultures were trypsinized to generate a single cell suspension. The single cell suspension was then cultured in the presence of embryoid body (EB) differentiation media (as described in the Examples) for a total of about 4 weeks. The cultures were passaged (with a 1:3 to 1:4 split using trypsin/EDTA) every 3-4 days. At the end of the differentiation period, the cells were tested for the presence of starting H1.1OGN cells by G418 resistance and/or by green fluorescence. The resulting cell line which is referred to herein as dH1.1f is maintained in alpha-MEM containing 10% inactivated fetal serum. The cell line is negative for both GFP and neomycin resistance.

The invention provides a variety of mutant iPS cell lines also. These mutant cell lines are generated from differentiated cells from human subjects having a condition known to have a genetic basis, and in some cases a clearly defined genetic basis. These lines therefore are referred to herein for example as cells (or cell lines) that comprise a particular mutation or mutations such as for example a Down syndrome mutation, a Duchenne type muscular dystrophy mutation, etc. These mutations are known in the art and reference can be made to any genetic analysis text or reference. It will be understood by those of ordinary skill in the art that depending on the mutation, some lines will harbour one mutation while others will harbour two mutations. Lines may harbour more than two mutations, but usually one or two mutations are necessary for manifestation of the disease phenotype. Thus some lines will harbour only one mutation and this mutation alone will be sufficient to manifest the condition in the subject harbouring the mutation. Such mutations are referred to as dominant mutations. In these lines, the other allele of the afflicted gene may be completely normal, but its presence is not sufficient to dampen the effects of the mutant allele. Other lines will harbour two mutations that may be identical or may be different. The end result of these mutations is that together they result in a mutant phenotype in the subject carrying both mutations. Example 3 provides various examples of lines that carry different mutations in the two copies of the affected gene (i.e., the alleles of that gene). Such lines may be referred to as compound heterozygotes since the alleles carry different mutations (and are thus heterozygous) that contribute to the mutant phenotype.

The presence of one or mutations at the iPS stage may not be immediately apparent but may be confirmed through molecular genetic analyses such as Southern blots, karyotyping (for example for trisomy 21), PCR, restriction fragment length polymorphisms, and the like. Those of ordinary skill in the art will be familiar with the techniques used to identify the various mutations described herein or otherwise associated with the conditions described herein. Similarly, those of ordinary skill in the medical arts including most notably medical practitioners will be able to readily diagnose a subject having any of the conditions recited herein, such that subjects having any of these conditions can be readily identified and their differentiated cells (including fibroblasts or mesenchymal cells) may be used to generate patient specific iPS cells.

The Examples describe the genetic mutations present in the differentiated cells and the iPS cells derived therefrom for a number of conditions. For example, mutant iPS cells comprising ADA-SCID mutations are generated in Example 3 and are characterized as being compound heterozygotes that have one ADA allele that comprises a GGG to GAA transition mutation in exon 7 that results in the G216R amino acid substitution and another ADA allele that comprises a frameshift deletion (-GAAGA) in exon 10. It should be understood however that the invention contemplates iPS that carry other mutations such as other ADA-SCID mutations such as but not limited to mutations that result in G74C, V129M, G140E, R149W, Q199P, 462delG, E337del, R211H, R156H, and P126Q amino acid mutations.

Similarly, the invention contemplates iPS cells that comprise one, two or more SBDS mutation(s) such as those described in Example 3 as well as those listed in the mutation registry for Shwachman syndrome (SBDSbase). Examples of such mutations include but are not limited to IVS2+2 T>C, IVS3-1 G>A, 183-184TA>CT(K62X), 119delG, and 505C>T (R169C). (Austin et al. 2005.)

The invention further contemplates iPS cells that comprise Down syndrome mutations such as those described in Example 3 including trisomy 21 (i.e., a karyotype having three copies of chromosome 21).

The invention contemplates iPS cells that comprise one, two or more Parkinson's disease mutations. Such mutations include mutations in the PARK1, PARK2, PARK3, PARK4, PARK 5, PARK 6, PARK 7 and/or PARK 8 genes (as described by Foltynie et al., 2002), the monoamine oxidase B gene, the N-acetyl transferase 2 detoxification enzyme, the glutathione transferase detoxification enzyme T1, or the tRNA Glu mitochondrial gene.

The invention contemplates iPS cells that comprise one, two or more Huntington disease mutations such as those described in Example 3. Such mutations are commonly present in the huntington gene which codes for the Huntington protein. The mutations generally introduce repeated glutamine coding codons into the gene sequence thereby resulting in a protein that has polyglutamine tracts. Sequences that result in less than 27 glutamines are considered normal, while those that result in 27-35 glutamine repeats show intermediate phenotype, those that result in 36-39 glutamines are associated with reduced penetrance, and finally those having more than 39 glutamines are associated with full penetrance.

The invention contemplates iPS cells that comprise one, two or more Duchenne type or Becker type muscular dystrophy mutations such as those described in Example 3. Other examples of such mutations include deletions in one or more of the exons 3-6, 8, 12, 13, 17, 19, 32-34, 43-48, 50, 51 and 60 of the dystrophin gene.

The invention contemplates iPS cells that comprise one, two or more Pearson syndrome mutations. Examples of such mutations include deletions of mitochondrial DNA (mtDNA) ranging from 1 to 10 kb in length. One of the more common mutations is a 4977 by deletion.

The invention contemplates iPS cells that comprise one, two or more Kearns-Sayre syndrome mutations. Examples of such mutations include deletions of mitochondrial DNA (mtDNA) ranging from 1 to 10 kb in length.

The invention contemplates iPS cells that comprise one, two or more retinoblastoma mutations. Examples of such mutations include deletions of the Rb-1 gene resulting in deletion of Rb-1 gene product function. The gene exists on chromosome 13, specifically at 13q 14.1-14.2. A variety of mutations have been observed associated with retinoblastoma including splicing errors, point mutations, small deletion in the gene promoter region, and the like. Specific mutations include but are not limited to deletions of 13q14, and translocations such as t(6:11) (q13:q25), t(12:13) (q23:q33), t(1:13) (p22:q12), and t(13:4) (q14:p16.3).

The invention contemplates iPS cells that comprise one, two or more Dyskeratosis congenita mutations. Examples of such mutations include mutations in the DKC1 gene that encodes dyskerin, or the TERC and/or TERT genes having gene products involved in telomere length.

The invention contemplates iPS cells that comprise one, two or more Gaucher disease mutations such as those described in Example 3. Thus, such mutations include genetic changes in the glucocerebrosidase gene that result in the following amino acid changes in the encoded gene: N370S, V395L, R120W, R48W, F37V, L444P, G46E, N188S, F2131I, V15L. The genetic mutations further include a G insertion resulting in 84GG, a C to T mutation at cDNA position 475 (yielding the R120W amino acid substitution), a C to T mutation at cDNA position 259 (yielding the R48W amino acid substitution), a T to G mutation at cDNA position 226 (yielding the F37V amino acid substitution), and the A to G mutation at cDNA position 1226.

The invention further provides methods for identifying additional factors that promote production of hiPS cells from differentiated human cells. These screening methods can be performed using any population of differentiated human cells. However, to minimize variability between test groups and between test and control groups, it is preferable to use a homogeneous population of differentiated cells. Thus, while primary cells can be used, it may be preferable in some instances to use cell lines, such as for example the dH1.1f cell line described herein. This line represents a homogeneous population of mature differentiated fibroblasts and thus it acts as a surrogate for primary fibroblasts harvested from an adult human. Another line that is useful in this regard is the MRC5 fetal fibroblast line discussed herein. Yet another cell population that can be used is adult fibroblasts such as the hFib2 cells described herein. Mesenchymal stem cells may also be used as the starting population in other embodiments.

As used herein, a factor that promotes production of hiPS cells is a factor that improves the yield of hiPS cells, whether quantitatively or qualitatively. As used herein, a candidate factor is a moiety that is being tested for its ability to promote hiPS cell production from differentiated human cells. It may be a chemical compound whether naturally occurring or not, a peptide or protein including but not limited to transcription factors, chromatin remodeling factors, and the like, or a nucleic acid including but not limited to an antisense nucleic acid or a short interfering nucleic acid (e.g., miRNA). Each of these factor classes may be generated as a library. Libraries facilitate the generation and screening of hundreds or thousands of candidates. The libraries themselves may comprise naturally occurring and/or non-naturally occurring members. The libraries may be small molecule libraries, transcript libraries, peptide libraries, and the like. It will be understood that in some instances screening of proteins and/or peptides may require the use of a library of nucleic acids that encode the candidate proteins or peptides.

The invention provides various screening methods. One screening method comprises ectopically expressing SOX2 and OCT4 in differentiated human cells in the presence or absence of a candidate factor, then culturing the cells under culture conditions and for a time sufficient for detection of hiPS cells, and measuring and comparing the yield of hiPS cells produced in the presence and absence of the candidate factor. A yield of hiPS cells produced in the presence of the candidate factor that exceeds the yield in the absence of the candidate factor indicates that the candidate factor promotes hiPS cell production.

The cells may also ectopically express MYC nucleic acid in combination with the SOX2 nucleic acid and the OCT4 nucleic acid.

In a retroviral context, the candidate factor may be present at the time of infection, or following infection. In the instance that the candidate factor is provided as a nucleic acid that encodes a protein or peptide, the nucleic acid may be ectopically expressed in the differentiated human cells in combination with the SOX2 and OCT4 nucleic acids. The nucleic acids (and their gene products) may be of mouse or human origin.

The culture conditions for such screening assays are similar to those recited herein, and thus in some instances include culturing in the presence of a ROCK inhibitor (e.g., Y27632).

Another screening method comprises ectopically expressing an OCT4 nucleic acid and a MYC nucleic acid in differentiated human cells in the presence and absence of a candidate factor, then culturing the cells under culture conditions and for a time sufficient for detection of hiPS cells, and measuring and comparing yield of hiPS cells produced in the presence and absence of the candidate factor. A yield of hiPS cells produced in the presence of the candidate factor that exceeds the yield in the absence of the candidate factor indicates a candidate factor that promotes hiPS production. In the instance that the candidate factor is provided as a nucleic acid that encodes a protein or peptide, the nucleic acid may be ectopically expressed in the differentiated human cells in combination with the OCT4 and MYC nucleic acids. The nucleic acids (and their gene products) may be of mouse or human origin.

The invention also contemplates screening methods that require the differentiation of the mutant iPS cells provided by the invention. The ability to differentiate such cells provides an opportunity not previously available to study the effects of one or more genetic mutations on differentiation. Thus, while an analysis of a human subject having a particular mutation and associated condition provides information relating to the final phenotype caused by the mutation, it often does not yield information about where the mutation manifests its effects during differentiation. By differentiating the mutant iPS cells provided by the invention into one or more lineages, the particular stages of differentiation affected by the mutation should be readily identified.

Moreover, such differentiation assays, whether in vitro or in vivo, also provide the platform from which therapies for each of the conditions can be tested. Such therapies may be gene therapies, or small molecule therapies, or some combination thereof, although they are not so limited. The differentiative profile of mutant iPS cells may be analyzed and preferably quantitated (e.g., via enumeration of cells of a given phenotype) in the presence or absence of a candidate molecule. In most cases, a change in a differentiative profile that resembles a normal profile (more so than a mutant profile) is indicative of a candidate molecule that should be pursued.

The hiPS cells may be provided as pharmaceutical compositions, together with a pharmaceutically acceptable carrier. The hiPS cells may be provided as a frozen aliquot of cells, or a culture of cells, possibly including MEFs also. In some instances the hiPS will be a clonal population. The iPS cells may also be provided as part of a cell population comprising cells that are the differentiated progeny of the iPS cells. The iPS cells may be identified by the presence of the retroviral or other ectopic expression constructs used to express the factor cocktails used to dedifferentiate the differentiated cells into iPS. They may also be recognized by expression of SOX2, OCT4 and/or KLF4 from endogenous loci rather than from the infected retroviral construct and loci contained therein.

As used herein, a pharmaceutically-acceptable carrier means a non-toxic material that does not interfere with the effectiveness of the biological activity of the active ingredients. Pharmaceutically acceptable carriers include diluents, fillers, salts, buffers, stabilizers, solubilizers and other materials which are well-known in the art. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The hiPS cells may be formulated for intravenous administration or alternatively as part of an implant.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

The following Examples demonstrate an experimental protocol for generating iPS cells from human differentiated cells. The human differentiated cells are provided either as fibroblasts differentiated from a human embryonic stem cell or as the human fetal fibroblast line MRC5.

These Examples show that expression of the transcription factors OCT4 and SOX2 together with either MYC or KLF4 are sufficient to reprogram fibroblasts differentiated from human ES cell lines and fibroblasts isolated from human fetal lung. The hiPS cells express markers characteristic of hES cells, form well-differentiated teratomas in immune-deficient mice, and can be differentiated into embryoid bodies in vitro. These data suggest that defined genetic factors are able to reprogram fetal human cells to pluripotency.

### Example 1

### Methods

Cell culture. H1.1 hES cells expressing GFP and Neo integrated into the OCT4 locus (H1.1OGN) were cultured in standard hES cell culture medium (DMEM/F12 containing 20% KOSR, 10 ng/ml of human recombinant bFGF, 1×NEAA, 5.5 mM 2-ME, 50 units/ml penicillin and 50 μg/ml streptomycin). H1.1OGN cells were split into differentiation medium (DMEM containing 15% IFS, 1 mM sodium pyruvate, 4.5 mM monothiolglycerol, 50 μg/mL ascorbic acid, 200 μg/mL iron-saturated transferrin, and 50 units/ml penicillin and 50 ug/ml streptomycin) for 4 weeks, with passaging every 3 to 4 days with 0.25% trypsin/EDTA. Differentiated H1.1OGN fibroblasts (dH1.1fs) were maintained in alpha-MEM containing 10% IFS. hFib2, MRC5 (purchased from ATCC), and 33Y (PT 2501, purchased from Lonza) were cultured in alpha-MEM containing 10% IFS.
Retroviral production and hiPS cell induction. Human OCT4, SOX2, and KLF4 were cloned by inserting cDNA produced by PCR into EcoRI and XhoI sites in pMIG vector (Van Parijs et al., 1999). pMIG expressing c-MYC was generously provided by Dr. Cleveland of St. Jude Hospital (Eischen et al., 2001). 293T cells in 10 cm plates were transfected with 2.5 μg of retroviral vector, 0.25 μg of VSV-G vector and 2.25 μg of Gag-Pol vector using FUGENE 6 reagents. Two days after transfection, supernatants were filtered through 0.45 μm cellulose acetate filter, centrifuged at 23,000 rpm for 90 min and stored −80 C until use. Lentivirus expressing dTomato was kindly provided by Niels Geijsen (Massachusetts General Hospital). 1×10⁵ dH1.1fs were plated in one well of six well plate and infected with retrovirus together with protamine sulfate. After three days of infection, dH1.1fs were split into plates pre-seeded with mouse embryonic fibroblasts (MEF). Medium was changed to hES culture medium 7 days post infection. 50 μg/ml of G418 were added after two weeks of infection to select hiPS cells.
Surface antigen staining. H1.1OGN, dH1.1f, hiPS, and human fibroblasts were fixed with 4% paraformaldehyde for 5 min (for alkaline phosphatase) or 30 min and stained for alkaline phosphatase, OCT4, NANOG, SSEA3, SSEA4, Tra-1-60 and Tra-1-80 according to the manufacturer's protocol.
RT-PCR, Southern blot, bisulfate sequencing and teratoma injection. RNAs from H1.1OGN, dH1.1fs, and hiPS were isolated using RNeasy kit (Qiagen) according to manufacturer's protocols. After RT reaction with oligo-dT, PCR was performed with primer sets:

| | |
|---|---|
| ACTB forward TGAAGTGTGACGTGGACATC, | (SEQ ID NO: 9) |
| ACTB reverse GGAGGAGCAATGATCTTGAT, | (SEQ ID NO: 10) |
| OCT4 forward AGCGAACCAGTATCGAGAAC, | (SEQ ID NO: 11) |
| OCT4 reverse TTACAGAACCACACTCGGAC, | (SEQ ID NO: 12) |
| NANOG forward TGAACCTCAGCTACAAACAG, | (SEQ ID NO: 13) |
| NANOG reverse TGGTGGTAGGAAGAGTAAAG, | (SEQ ID NO: 14) |
| XIST forward GTCATCACAACAGCAGTTCT, | (SEQ ID NO: 15) |
| and | |
| XIST reverse GACTACTAAGGACACATGCA. | (SEQ ID NO: 16) |

For Southern blot, genomic DNA (gDNA) was isolated using DNeasy kit according to manufacture's protocol, and digested with SpeI. The presence of integrated virus was identified by hybridizing blots with probes recognizing OCT4, SOX2, KLF4, and MYC. Southern blots were also performed for the presence of lentivirus expressing dTomato using a dTomato specific probe.

Bisulfite treatment of gDNA was carried out using a Chemicon CpGenome DNA Modification Kit according to the manufacturer's protocol. Nested PCR was then used to amplify OCT4 and NANOG promoters (Freberg et al., 2007). PCR products were cloned from two independent PCR reactions and resulting individual clones were sequenced.

For teratoma formation, 1×10⁶ cells of H1.1OGN, dH1.1fs and hiPS cells were resuspended in DMEM, Matrigel and collagen mixture (2:1:1 volume ratio) and injected intramuscularly into immune-compromised Rag2gammaC−/− mice.

Microarray analysis. Total RNA from H1.1OGN, dH1.1fs, and hiPS cells was isolated and processed for hybridization with Microarray according to manufacturers' protocols. Data was analyzed by using GeneSpring (Agilent).

### Results

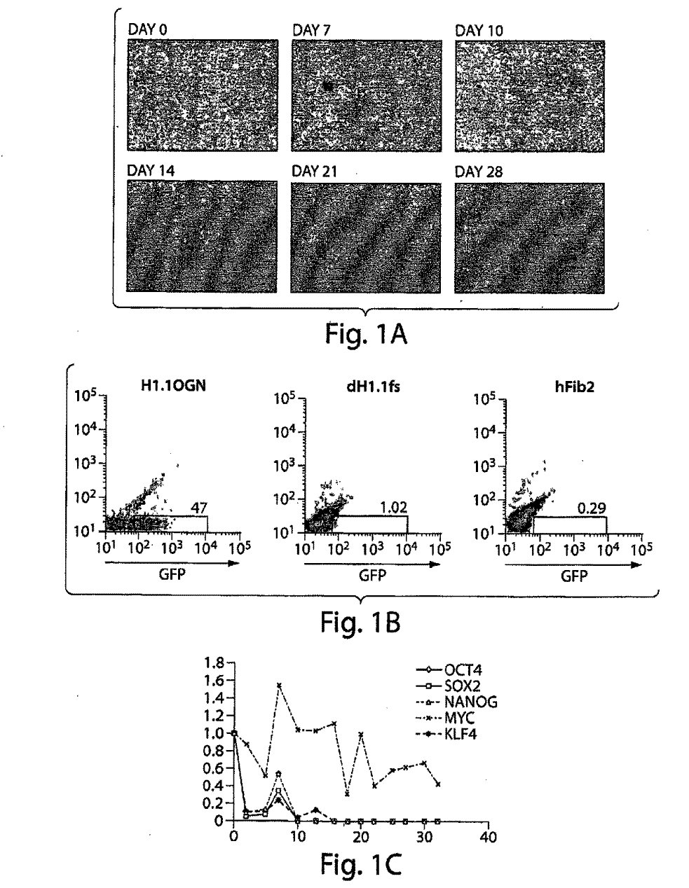

Generation of human fibroblasts with a reporter of pluripotency. In order to facilitate the isolation of iPS colonies from differentiated human fibroblasts, the previously described H1.1 hES cells that carry the GFP and Neo genes integrated into the OCT4 locus (H1.1OGN; Zwaka and Thomson, 2003) were used. H1.1OGN cells express GFP and show neomycin resistance only in the undifferentiated state. H1.1OGN cells were differentiated in vitro for four weeks, resulting in a homogeneous population of fibroblast-like cells, which were named dH1.1f cells. GFP expression was undetectable in dH1.1f cells, as assayed by flow cytometry (FIG. 1B). No differentiated dH10.1f cells survived selection in G418 (50 μg/ml). Expression of OCT4, NANOG (FIG. 1C) and REX1 (data not shown) was dramatically reduced in dH1.1f cells. The methylation status of the OCT4 and NANOG loci in dH1.1f cells was determined using bisulfite sequencing. While H1.1OGN showed largely unmethylated sequences, the OCT4 and NANOG loci in dH1.1f cells were highly methylated. No tumors formed following injection of dH1.1f cells into immune-compromised mice, in contrast to the parental H1.1OGN cells, which readily formed teratomas. Taken together, these data establish that dH1.1f cells represent a differentiated population that has lost the essential features of pluripotency.

To eliminate the possibility of contamination from residual undifferentiated hES cells in dH1.1f cultures, the population was infected with a lentiviral construct carrying dTomato, and individual colonies generated on plates by serial dilution were picked and expanded. Southern hybridization confirmed a single integration of the lentivirus in dH1.1cf (cloned fibroblast) cells, thereby confirming their derivation from a single clone. The dH1.1cf cells were GFP-negative, G418 sensitive, did not express OCT4 or NANOG protein, and failed to induce tumors in immune-deficient mice.

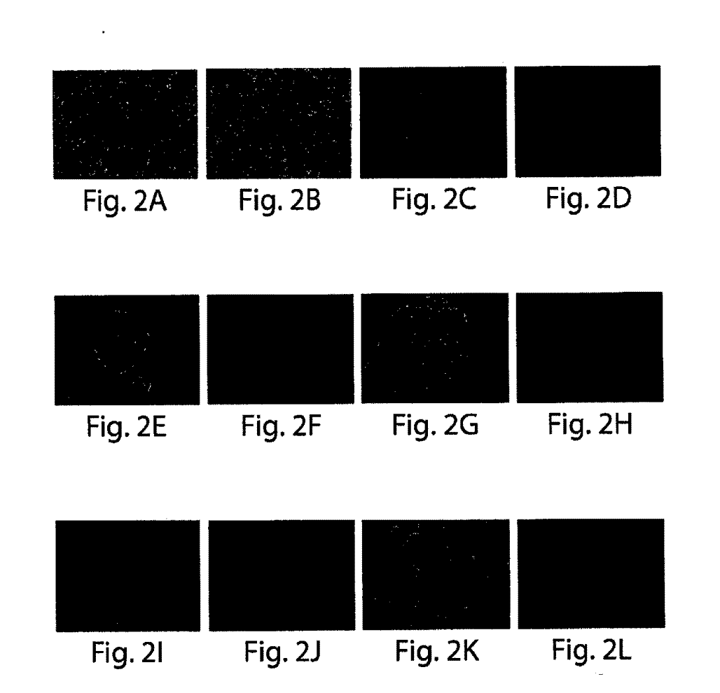

Generation of human induced pluripotent stem (hiPS) cells. Cultures of dH1.1f and cloned dH1.1cf cells were infected with retroviral supernatants carrying OCT4, SOX2, KLF4, and MYC. Three or four days after infection, cells were split into plates with MEFs and further cultured. Seven days infection, cells were cultured in hES cell culture medium supplemented with the ROCK inhibitor Y27326, previously shown to enhance survival and clonogenicity of single dissociated hES cells (Watanabe et al., 2007). Twelve days after infection, G418 was added to the cultures to select for cells that had re-activated the OCT4 locus. G418-resistant colonies with an ES-like morphology appeared, and were picked and expanded. Cultures showed a morphology indistinguishable from the parental H1.1OGN cells (FIG. 2). A yield of approximately 100 G418-resistant colonies was observed per infection of 1×10⁵ dH1.1f cells with the four factors, consistent with a reprogramming efficiency of 0.1%. Infection of different clones of dH1.1cf cells yielded a variation frequency of 2-100 colonies, suggesting clonal variation in susceptibility to OCT4 reactivation. Cultures of G418-resistant colonies from dH1.1cf clones, which were deemed hiPS cells, carried the identical lentiviral integration site as the parental clone, thereby confirming their derivation from the dH1.1cf clone, and ruling out the possibility that a contaminating H1.1OGN cell had been recultured.

hiPS cells have a similar growth profile as H1.1OGN cells, and showed a similar cell cycle profile, with the short G1 phase that is characteristic of hES cells (Fluckiger et al., 2006)). HiPS cells show normal karyotypes. They also expressed hES cell-specific pluripotent proteins, OCT4 and NANOG, and surface markers, SSEA3, SSEA4, Tra-1-60 and Tra-1-80 together with alkaline phosphatase (FIG. 2). Quantitative PCR analysis of viral transgenes demonstrated reduced expression relative to the endogenous loci, suggesting that the viruses had undergone transcriptional silencing, despite their multiple integrations. Expression of the endogenous OCT4 and NANOG loci were equivalent in hiPS and H1.1OGN cells, and global gene expression analysis by microarray showed highly similar patterns of gene expression. The hiPS cells formed teratomas in immune-compromised mice, containing differentiated tissue from all three embryonic germ layers.

The methylation status of the OCT4 and NANOG promoters was assessed in hiPS cells and hES cells by bisulfite sequencing. While the promoters of OCT4 and NANOG are largely methylated in dH1.1f cells, those of iPS and hES cells show demethylation, suggesting the reactivation of OCT4 and NANOG loci.

Transduction of defined factors into fetal, neonatal, and adult human fibroblasts. Genetic selection for the reactivated OCT4 or NANOG locus is not required when isolating iPS cells from the mouse, as selection of colonies based on ES-like morphology alone is sufficient to identify fully reprogrammed clones (Meissner et al., 2007) Likewise, hiPS cells were readily isolated from dH1.1f fibroblasts by morphology alone. Therefore, OCT4, SOX2, KLF4, and MYC were introduced into human somatic cells from developmentally diverse stages and isolation of hiPS cells by morphologic assessment was attempted.

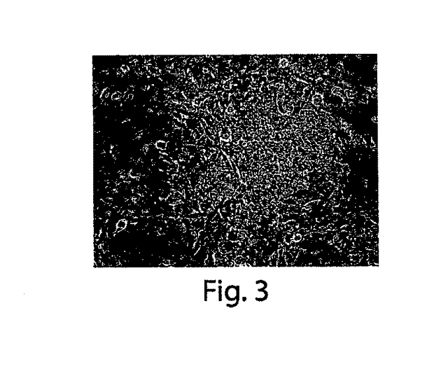

It is well known that, compared to rodent cells, human cells acquire distinct genetic lesions during immortalization and tumorigenesis. Therefore, an attempt was made to supplement the four factors (OCT4, SOX2, KLF4 and MYC) with genes known to play a role in establishing human cells in culture. These candidates included the catalytic subunit of human telomerase, hTERT, separately or together with SV40 Large T, which has potent anti-apoptotic activity and is permissive for transforming human fibroblasts to tumorigenicity. (Hahn et al., 1999) When hTERT and SV40 LT were introduced together with the four transcription factors into hFib2 fibroblasts, cultures grew more rapidly and there was cellular loss and sloughing of cells into the media. Although we originally believed that the colony morphology was clearly distinct from hES cell colonies, a closer retrospective analysis revealed that these colonies were indeed iPS cell colonies as shown in FIG. 3. These colonies had been observed prior to November 2007. Similar results were obtained by ectopically expressing the same six factor cocktail in BJ1 (neonatal foreskin fibroblasts) and MRC5 (primary fetal lung fibroblasts) cells, although MRC5 cells also appear to give rise to hiPS-like cells when infected with only four factors (i.e., OCT4, SOX2, KLF4 and MYC).

### Conclusions

These experiments demonstrate that differentiated derivatives of hES cells (as well as fetal lung fibroblasts) which lack the essential features of pluripotency can be reprogrammed to iPS cells by the same factors that were successful in the mouse: OCT4, SOX2, KLF4 and MYC. Similarly, adult fibroblasts such as adult dermal fibroblasts can be reprogrammed to iPS cells by the same four factors, either alone or together with hTERT and SV40 large T. A comparison of relative gene expression profiles and epigenetic modifications between adult human fibroblasts and dH1.1f cells will be instrumental in identifying additional candidates that might be required to reprogram adult cells. Our results establish the feasibility of reprogramming of human cells with defined factors.

### Example 2

### Abstract

Pluripotency pertains to the cells of early embryos that can generate all of the tissues in the organism. Embryonic stem cells are embryo-derived cell lines that retain pluripotency and represent invaluable tools for research into the mechanisms of tissue formation. Recently, murine fibroblasts have been reprogrammed directly to pluripotency by ectopic expression of four transcription factors (Oct4, Sox2, Klf4 and Myc) to yield induced pluripotent stem (iPS) cells. Using these same factors, we have derived iPS cells from fetal, neonatal and adult human primary cells, including dermal fibroblasts isolated from a skin biopsy of a healthy research subject. Human iPS cells resemble embryonic stem cells in morphology and gene expression and in the capacity to form teratomas in immune-deficient mice. These data demonstrate that defined factors can reprogramme human cells to pluripotency, and establish a method whereby patient-specific cells might be established in culture.

### Methods

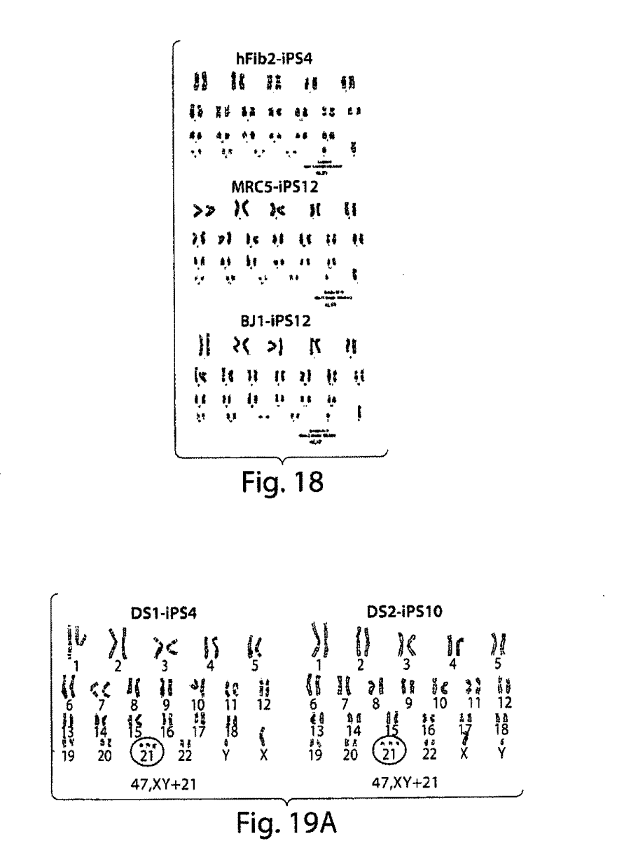

Cell culture. H1.1 human ES cells expressing GFP and neo integrated into the OCT4 locus (H1-OGN; Zwaka (2003)) were cultured in standard human ES cell culture medium (DMEM/F12 containing 20% KOSR, 10 ng ml⁻¹ of human recombinant basic fibroblast growth factor, 1×NEAA, 5.5 mM 2-ME, 50 units ml⁻¹ penicillin and 50 μg ml⁻¹ streptomycin). H1-OGN cells were split into differentiation medium (DMEM containing 15% IFS, 1 mM sodium pyruvate, 4.5 mM monothioglycerol, 50 μg ml⁻¹ ascorbic acid, 200 μg ml⁻¹ iron-saturated transferrin, and 50 units ml⁻¹ penicillin and 50 μg ml⁻¹ streptomycin) for 4 weeks, with passaging every 3 to 4 days with 0.25% trypsin/EDTA. Differentiated fibroblasts (dH1f) and clones (dH1cf) were maintained in alpha-MEM containing 10% IFS. The following cell lines were obtained from commercial vendors and cultured in alpha-MEM containing 10% IFS: MRC5 (fibroblasts isolated from normal lung tissue of a 14-week-old male fetus; ATCC), BJ1 (neonatal foreskin fibroblast; ATCC) and MSC (mesenchymal stem cells cultured from bone marrow of a 33-yr-old male; Lonza). To form embryoid bodies, confluent undifferentiated iPS cells were mechanically scraped into strips and transferred to 6-well, low-attachment plates in differentiation medium consisting of knockout DMEM (Invitrogen) supplemented with 20% fetal bovine serum (Stem Cell Technologies), 0.1 mM non-essential amino acids (Invitrogen), 1 mM L-glutamine (Invitrogen) and 0.1 mM β-mercaptoethanol (Sigma).
Derivation of primary human fibroblast lines (hFib2). Procurement of skin tissue for use in reprogramming experiments was obtained via informed consent under a protocol approved by the Institutional Review Board and the Embryonic Stem Cell Research Oversight Committee of Children's Hospital Boston. Using sterile technique, a 6-mm full-thickness skin punch biopsy was obtained from the volar surface of the forearm of a healthy volunteer male. The biopsy was cut into 2×2 mm pieces. The pieces were plated in a 6-well plate and were trapped under a sterile cover slip to maintain them in place. Human fibroblast derivation media consisted of DMEM (Invitrogen), 10% FBS (Invitrogen) and penicillin/streptomycin (Invitrogen). A dense outgrowth of cells appeared after 7-14 days, which were passaged using 0.25% trypsin EDTA.
Retroviral production and human iPS cell induction. Human OCT4, SOX2 and KLF4 were cloned by inserting cDNA produced by PCR into the EcoRI and XhoI sites of the pMIG vector (Van Parijs et al., 1999). pMIG expressing c-MYC was provided by J. Cleveland (Eischen et al., 2001). SV40 large T in the pBABE-puro vector (plasmid 13970, T. Roberts) and hTERT in the pBABE-hygro vector (plasmid 1773, R. Weinberg) were obtained from Addgene. 293T cells in 10-cm plates were transfected with 2.5 μg of retroviral vector, 0.25 μg of VSV-G vector and 2.25 μg of Gag-Pol vector using FUGENE 6 reagents. Two days after transfection, supernatants were filtered through 0.45 μm cellulose acetate filter, centrifuged at 23,000 r.p.m. for 90 min and stored at −80° C. until use. Lentivirus expressing dTomato was provided by N. Geijsen. 1×10⁵ of target somatic cells were plated in one well of a six-well plate and infected with retrovirus together with protamine sulphate. After 3 days of infection, cells were split into plates pre-seeded with mouse embryonic fibroblasts (MEFs). Medium was changed to human ES culture medium containing Y27632 7 days after infection. Chromosome counts of cell lines dH1f-iPS3-3, dH1cf32-iPS2, MRC5-iPS2, BJ-1-iPS1, BJ1-iPS3, MSC-iPS1 and hFib2-iPS1 all revealed a normal diploid number of 46. Normal karyotypes were documented for BJ1-iPS12, MRC5-iPS12 and hFib2-iPS4 (FIG. 18). The earliest cell line derived, dH1f-iPS3-3, has been maintained in continuous cell culture for over 5 months (30 passages).
Surface antigen staining. Cells were fixed in 4% paraformaldehyde for 30 min, permeabilized with 0.2% Triton X-100 for 30 min, and blocked in 3% BSA in PBS for 2 h. Cells were incubated with primary antibody overnight at 4° C., washed, and incubated with Alexa Fluor (Invitrogen) secondary antibody for 2 h. SSEA3, SSEA4, TRA-1-60 and TRA-1-81 antibodies were obtained from Millipore. OCT3/4 and NANOG antibodies were obtained from Abcam. Alkaline phosphatase staining was done per the manufacturer's recommendations (Millipore).
RT-PCR. RNA was isolated using an RNeasy kit (Qiagen) according to manufacturer's protocol. First-strand cDNA was primed via random hexamers and RT-PCR was performed with primer sets corresponding to Table 2. For quantitative RT-PCR, Brilliant SYBR green was used (Stratagene).
Bisulphite genomic sequencing. Bisulphite treatment of genomic DNA (gDNA) was carried out using a CpGenome DNA Modification Kit (Chemicon) according to the manufacturer's protocol. Sample treatment and processing were performed simultaneously for all cell lines, with the exception of dH1f. Converted gDNA was amplified by PCR using OCT4 primer sets 1, 4 and 7 (from Freberg et al., 2007; Deb-Rinker et al., 2005) and NANOG primer sets 1 and 2 (from Freberg et al., 2007). PCR products were gel purified and cloned into bacteria using TOPO TA cloning (Invitrogen). Bisulphite conversion efficiency of non-CpG cytosines ranged from 80% to 99% for all individual clones for each sample.
Microarray analysis. Total RNA was isolated from cells using RNeasy kit with DNase treatment (Qiagen). RNA probes for microarray hybridization were prepared and hybridized to Affymetrix HG U133 plus 2 oligonucleotide microarrays according to the manufacturer's protocols (processed by the Biopolymer facility of Harvard Medical School). Microarrays were scanned and data were analysed using GeneSpring GX7.3.1.
Fingerprinting analysis. PCR was used to amplify across discrete genomic intervals containing highly variable numbers of tandem repeats (VNTR) in order to verify the genetic relatedness of iPS cell lines relative to their parent fibroblasts. A total of 50 ng of genomic DNA was used per reaction, cycled 35 times through 94° C.×1 min, 55° C.×1 min, and 72° C.×1 min, and run on 2.5% agarose gels. Qualitative determinations were made based on differential amplicon mobility for each primer set: D10S1214, repeat (GGAA)ₙ, average heterozygosity 0.97; D17S1290, repeat (GATA)ₙ, average heterozygosity 0.84; D7S796, repeat (GATA)ₙ, average heterozygosity 0.95; and D21S2055, repeat (GATA)ₙ, average heterozygosity 0.88 (Invitrogen).
Southern hybridization. For Southern blots, gDNA was isolated using the DNeasy kit (Qiagen) according to the manufacturer's protocol, digested with XbaI (for dTomato), or SpeI and EcoRI (for OCT4 and SOX2) and separated via agarose gel electrophoresis. Transfer to nylon membranes (Nytran Supercharge, Schleicher & Schuell Bioscience) was completed overnight in 10×SSC. Probes were labelled with P-dCTP (Ready-to-Go DNA Labelling Beads, Amersham) and blots were hybridized (MiracleHyb, Stratagene) overnight to detect the presence of integrated viruses encoding dTomato, OCT4, or SOX2.
Assay for teratoma formation. For teratoma formation, 1×10⁶ cells were resuspended in a mixture of DMEM, Matrigel and collagen (ratio of 2:1:1) and injected intramuscularly into immune-compromised Rag^{-/-}/γc^{-/-} mice. Xenografted masses formed within 4 to 6 weeks and paraffin sections were stained with haematoxylin and eosin for all histological determinations.
Haematopoietic colony forming assays. Human iPS lines were differentiated for 14 days as embryoid bodies in culture media described above supplemented with SCF (300 ng ml⁻¹), Flt-3 ligand (300 ng ml⁻¹), IL-3 (10 ng ml⁻¹), IL-6 (10 ng m⁻¹), G-CSF (50 ng ml⁻¹) and BMP4 (50 ng ml⁻¹). Embryoid bodies were disassociated and plated into methylcellulose colony-forming assay media containing SCF, GM-CSF, IL-3 and Epo (H4434, Stem Cell Technologies) at a density of 25,000 cells ml⁻¹.
Karyotype analysis. Chromosomal studies were performed at the Cytogenetics Core of the Dana-Farber/Harvard Cancer Center using standard protocols for high-resolution G-banding.

### Results

Pluripotency can be induced in somatic cells by nuclear transfer into oocytes (Wakayama et al., 2001) and fusion with embryonic stem cells (Cowan et al., 2005), and for male germ cells by cell culture alone (Kanatsu-Shinohara et al., 2004). Ectopic expression of four transcription factors (Oct4, Sox2, Klf4 and Myc) in murine fibroblasts is sufficient to yield iPS cells that resemble embryonic stem (ES) cells in their capacity to form chimeric embryos and contribute to the germ lineage (Takahashi and Yamanaka, 2006; Wernig et al., 2007; Okita et al., 2007; Maherali et al., 2007). Direct, factor-based reprogramming might enable the generation of pluripotent cell lines from patients afflicted by disease or disability, which could then be exploited in fundamental studies of disease pathophysiology or drug screening, or in pre-clinical proof-of-principle experiments that couple gene repair and cell replacement strategies.

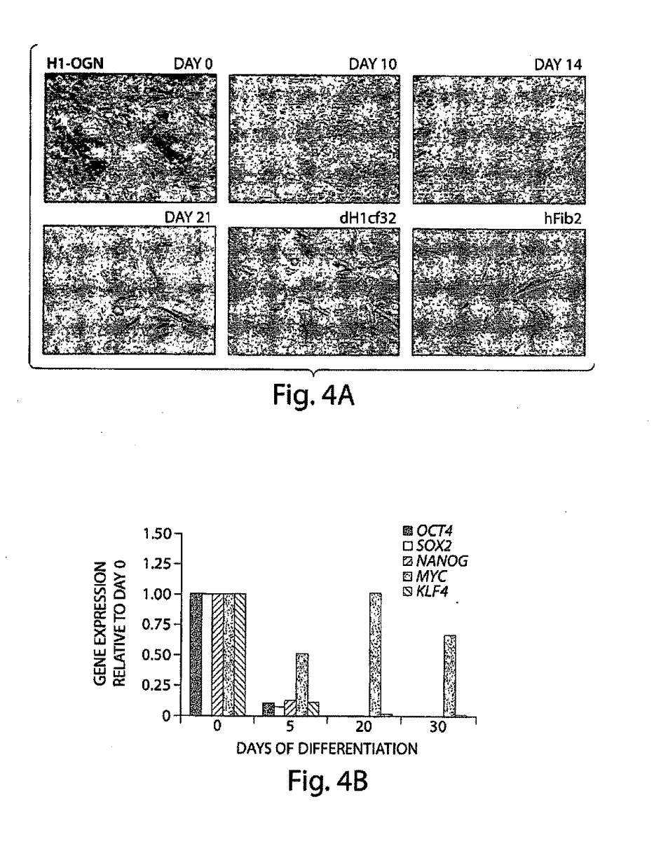

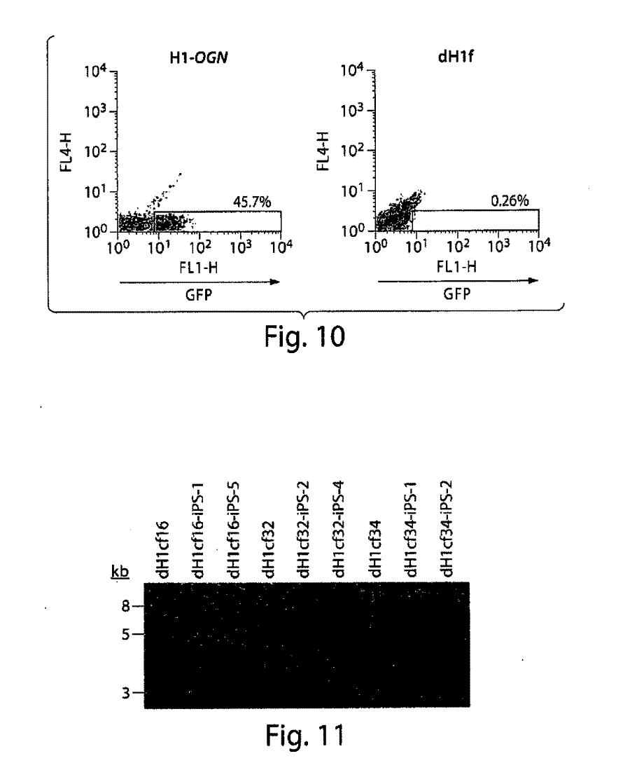

We attempted to use the original four reprogramming factors defined by Takahashi (2006) (OCT4, SOX2, KLF4 and MYC) to isolate iPS cells from human embryonic fibroblasts differentiated from H1-OGN cells, human ES cells that express the green fluorescence protein (GFP) reporter and neomycin (G418) resistance genes by virtue of their integration into the OCT4 locus by homologous recombination (H1-OGN; Zwaka and Thomson, 2003). We differentiated H1-OGN cells in vitro for 4 weeks, and propagated a homogeneous population of fibroblast-like cells (dH1f, differentiated H1-OGN fibroblast; FIG. 4A). GFP expression was undetectable in dH1f cells, as assayed by flow cytometry (FIG. 10). Expression of OCT4, SOX2, NANOG and KLF4 was extinguished in dH1f cells, whereas MYC expression persisted at near-comparable levels to undifferentiated H1-OGN cells (FIG. 4B). The dH1f cells could be cultured readily for at least 14 passages, after which their proliferation slowed markedly. No dH1f cells survived selection in G418 (50 ng ml⁻¹), and no tumours formed after injection of dH1f cells into immune-deficient mice. Taken together, these data establish that dH1f cells represent differentiated human ES cell derivatives that have lost the essential features of pluripotency.

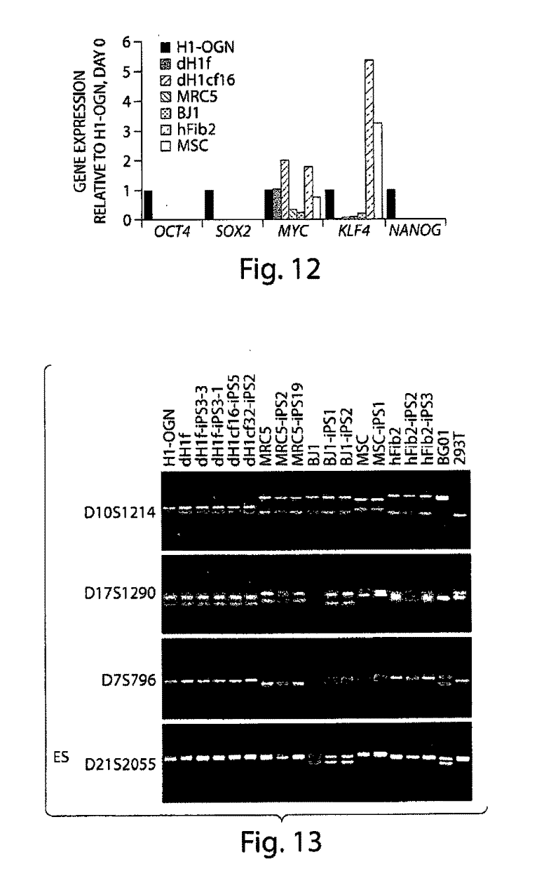

To ensure propagation of differentiated fibroblasts free of contamination by undifferentiated ES cells, we infected early passage dH1f cells with a lentiviral construct carrying the dTomato reporter gene, plated infected cells by serial dilution, and expanded individual colonies. Southern hybridization confirmed distinct single or double lentiviral integration sites in three cell lines, thereby confirming their clonal derivation from single cells (cloned dH1cf16, dH1cf32 and dH1cf34; FIG. 11). Proliferation of the cloned dH1cf cells began to slow markedly after an additional 4-5 passages. The dH1cf clones were G418 sensitive, negative for expression of GFP, OCT4 and NANOG, and failed to induce tumours in immunodeficient mice (FIG. 12 and data not shown).

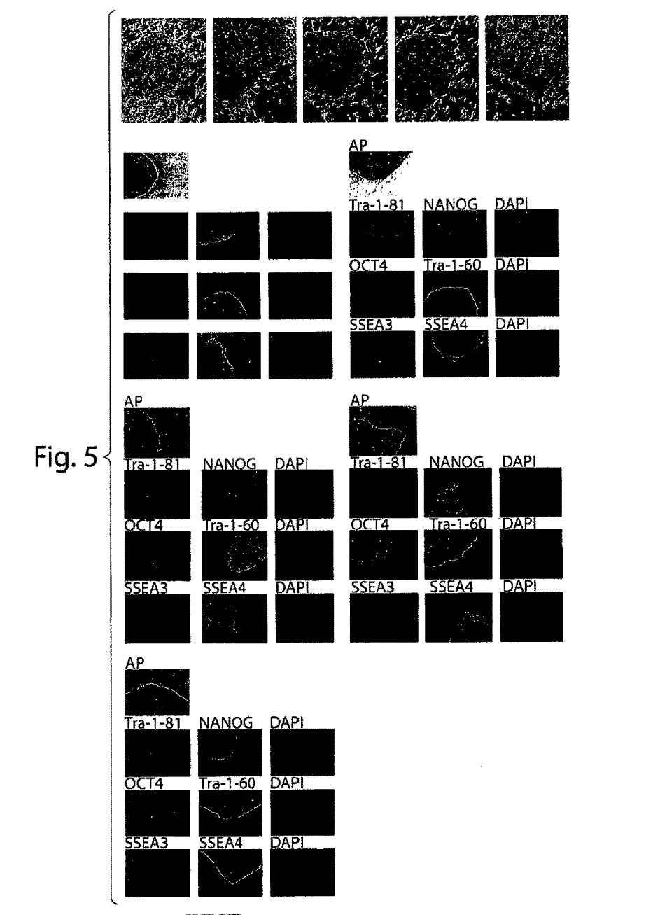

Reprogramming of human ES-cell-derived fetal fibroblasts. We infected cultures of dH1f and cloned dH1cf cells with a cocktail of retroviral supernatants carrying human OCT4, SOX2, MYC and KLF4. Seven days after infection, cells were plated in human ES cell culture medium supplemented with the ROCK inhibitor Y27632, previously shown to enhance survival and clonogenicity of single dissociated human ES cells (Watanabe et al., 2007). By 14 days after infection, cultures of infected dH1f cells showed distinct small colonies that were picked and expanded. The resulting cultures harboured colonies for which morphology was indistinguishable from the parental H1-OGN cells (FIG. 5A). Selection with G418 was not required to identify cells with ES-cell-like colony morphology; rather, morphology itself sufficed, as reported for identification of murine iPS cells (Meissner et al., 2007; Blelloch et al., 2007). We performed ten independent infections of 1×10⁵ dH1f cells with the four factors, and consistently observed approximately 100 human ES-cell-like colonies, for a reprogramming efficiency of ˜0.1% (Table 1). Surprisingly, we obtained human ES-cell-like colonies when we eliminated either MYC or KLF4 from the cocktails, although with markedly lower efficiency (Table 1). Infection of different clones of dH1cfs revealed a lower efficiency and delayed appearance of ES-cell-like colonies (between 6-47 colonies per 10⁵ cells after 21 days). Expanded cultures of human ES-cell-like colonies from dH1cf clones carried the identical lentiviral integration site as the parental cell line, thereby confirming their derivation from the original dH1cf clone, and eliminating the possibility that a contaminating undifferentiated H1-OGN cell had been re-isolated (FIG. 11).
Reprogramming of fetal, neonatal and adult fibroblasts We next tested a diverse panel of human primary cells available from commercial sources, as well as primary dermal fibroblasts isolated from a skin biopsy from a healthy volunteer, which were obtained following informed consent for reprogramming studies under a protocol approved by the Institutional Review Board and Embryonic Stem Cell Research Oversight Committee of Children's Hospital Boston.

We isolated cells with human ES-cell-like morphology from cultures of MRC5 fetal lung fibroblasts around 21 days after infection with the four transcription factors. We were also able to identify human ES-cell-like colonies by introduction of the four factors into Detroit 551 cells, another human primary cell culture derived from fetal skin (data not shown). In contrast to our results with human ES-cell-derived fibroblasts (dH1f, dH1cf) and primary fetal cells (MRC5, Detroit 551), transduction of the four transcription factors into more developmentally mature somatic cells, for example, neonatal foreskin fibroblasts (BJ1), adult mesenchymal stem cells (MSC) and adult dermal fibroblasts (hFib2), resulted in slowed proliferation and cellular senescence, and in these experiments we failed to identify colonies with obvious E-cell-like morphology from any of these infected cell cultures. We reasoned that adult human somatic cells might require additional factors to grow in continuous cell culture and to be reprogrammed to pluripotency, and thus we supplemented the four factors (OCT4, SOX2, MYC and KLF4) with genes known to have a role in establishing human cells in culture: the catalytic subunit of human telomerase, hTERT (Bodnar et al., 1998), and SV40 large T, which has potent anti-apoptotic activity (Hahn et al., 1999). When hTERT and SV40 large T were introduced together with the four transcription factors into BJ1, MSC and hFib2 cells, the cultures grew more rapidly but still showed significant cellular loss and sloughing into the media. However, against the background of adherent cells, we were able to recognize colonies with human ES-cell-like morphology (FIG. 5A and Table 1). Individual colonies of human ES-cell-like cells were picked and expanded. All ES-cell-like colonies shared DNA fingerprints with the line from which they derived, thereby ruling out the possibility of contamination with existing human ES cells being carried in the laboratory (FIG. 13).

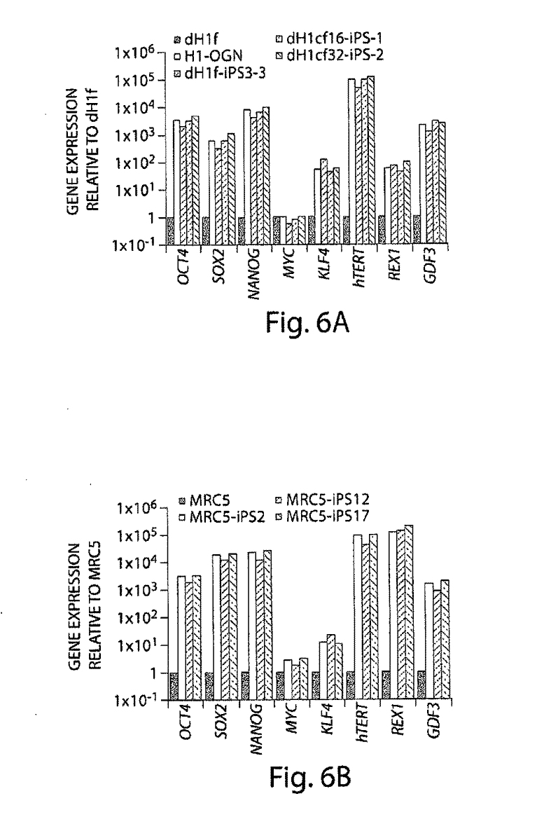

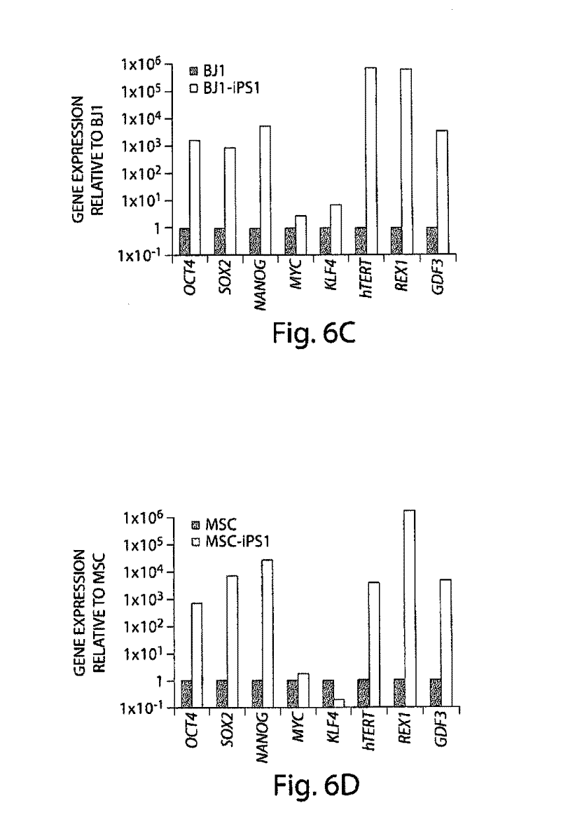

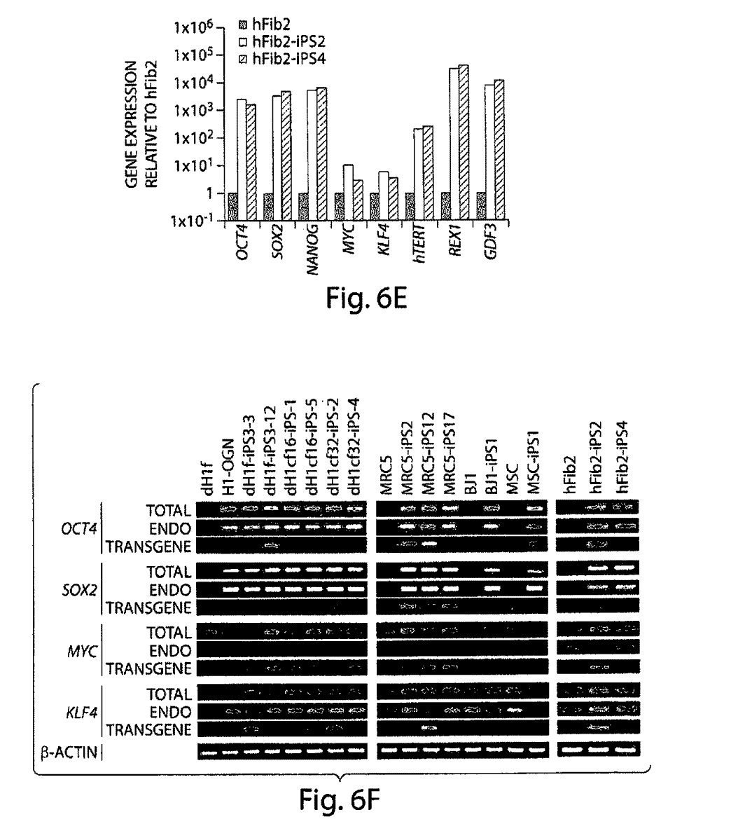

Characterization of reprogrammed somatic cell lines. We analysed colonies selected for human ES-cell-like morphology from dH1f, MRC5, BJ1, MSC and hFib2 by immunohistochemistry, and detected expression of alkaline phosphatase, Tra-1-81, Tra-1-60, SSEA3, SSEA4, OCT4 and NANOG (FIG. 2B-F), all markers shared with human ES cells (Adewumi et al., 2007). We also analysed gene expression by quantitative polymerase chain reaction (PCR) analysis, and noted that for derivatives of dH1f, dH1cf, MRC5, BJ1, MSC and hFib2, expression of OCT4, SOX2, NANOG, KLF4, hTERT, REX1 and GDF3 was markedly elevated over the respective fibroblast population, and comparable to the parental H1-OGN human ES cells (FIG. 6A-E). Expression of MYC did not vary markedly from the parental cell lines, suggesting that a consistent expression level was required to sustain cell proliferation in multiple cell types under our culture conditions (FIG. 6A-E). In murine iPS cells, retroviral expression of murine Oct4, Sox2, Myc and Klf4 is silenced during iPS derivation and complemented by reactivation of expression from the endogenous gene loci (Takahashi and Yamanaka, 2006; Wernig et al., 2007; Okita et al., 2007; Maherali et al., 2007).

TABLE 1
ES-cell-like colony formation with various donor cells and reprogramming factors

| Cell line | OCT4 and SOX2 | Three factors | Four factors | Six factors‡ |
|---|---|---|---|---|
| ES-cell-derived fibroblasts dH1f | 0 | -OCT4*, 0; -SOX2†, 0; | 118 ± 35 | 250 |
| | | -KLF4, 63; -MYC, 11 | | |
| ES-cell-derived fibroblasts dH1cf | ND | ND | dH1cf16, 47; | d |
| (clones 16, 32, 34) | | | dH1cf32, 12; | dH1cf32, 40; |
| | | | dH1cf34, 6 | dH1cf34, 17 |
| Fetal lung fibroblasts MRC5 | ND | ND | 39 | ND |
| Neonatal foreskin fibroblasts BJ1 | ND | ND | 0 | 21 |
| Mesenchymal stem cells | ND | ND | 0 | 3 |
| Adult dermal fibroblasts hFib2 | ND | ND | 0 | 7 |

The four factors were OCT4, SOX2, MYC and KLF4; the six factors were OCT4, SOX2, MYC, KLF4, hTERT and SV40 large T. Numbers are for colonies showing human ES-cell-like morphology per 10⁵ infected cells.
ND, not determined.
*No human ES-cell-like colonies but numerous (~10²) colonies with flat morphology were observed.
†No colonies observed, not even the flat variety seen with the three-factor combination lacking OCT4.
‡Only human ES-cell-like colonies scored, despite observation of frequent flat colonies.

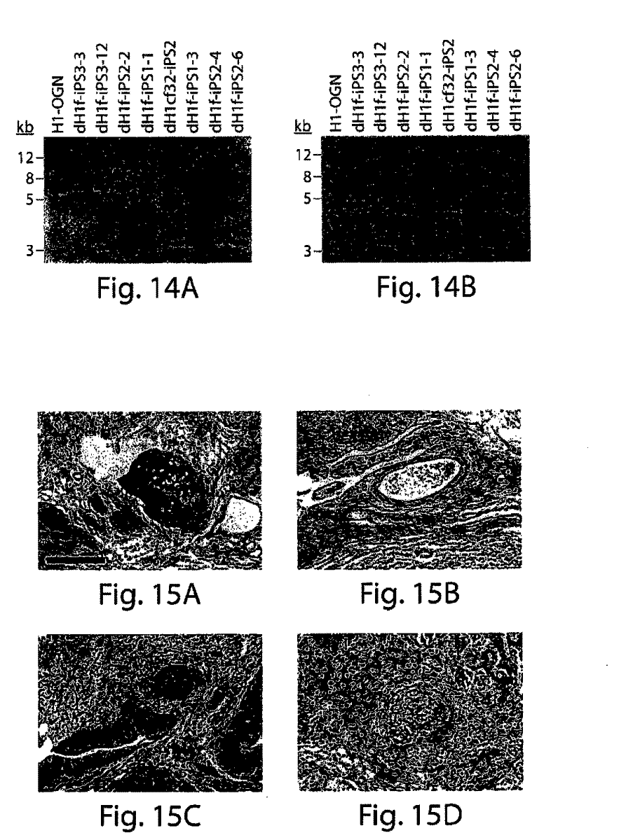

We analysed the expression of the endogenous loci and retroviral transgenes, and found that total expression of OCT4, SOX2, MYC and KLF4 was comparable to human ES cells (FIG. 6F). Expression of the endogenous OCT4 and SOX2 loci was consistently upregulated relative to parental cells, and accompanied by variable levels of retroviral transgene expression, with silencing in some cells (FIG. 6F). These data suggest that expression of OCT4 and SOX2 is titrated to a specific range during selection in cell culture. There was variable but persistent expression of the retroviral MYC and KLF4 transgenes (FIG. 6F). Single or multiple integrations (2-6 copies) of the OCT4 and SOX2 transgenes were detected by Southern blot analysis in different cell lines (FIG. 14A, B).

We were successful in recovering human ES-cell-like colonies from the postnatal BJ1, MSC and hFIB2 cells only when we used six factors in our retroviral cocktail (adding hTERT and SV40 large T to the original four factors). Although PCR analysis of genomic DNA from the bulk early post-infection cultures detected the respective retroviruses, the human ES-cell-like colonies that we ultimately isolated failed to show integration or expression of hTERT and SV40 large T (data not shown). We thus conclude that hTERT and SV40 large T are not essential to the intrinsic reprogramming of the recovered ES-cell-like cells. Because the six-factor cocktail showed a higher frequency of human ES-cell-like colony formation in all cell contexts tested (Table 1), these factors may act indirectly on supportive cells in the culture to enhance the efficiency with which the reprogrammed colonies can be selected.

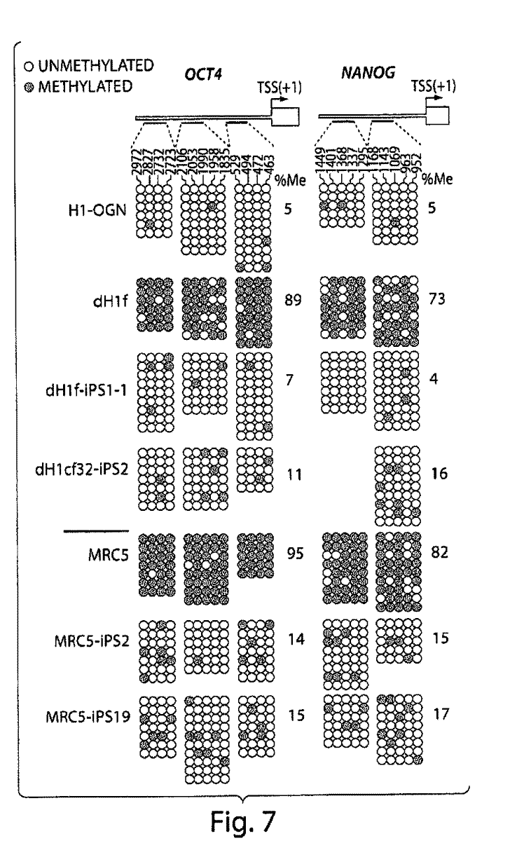

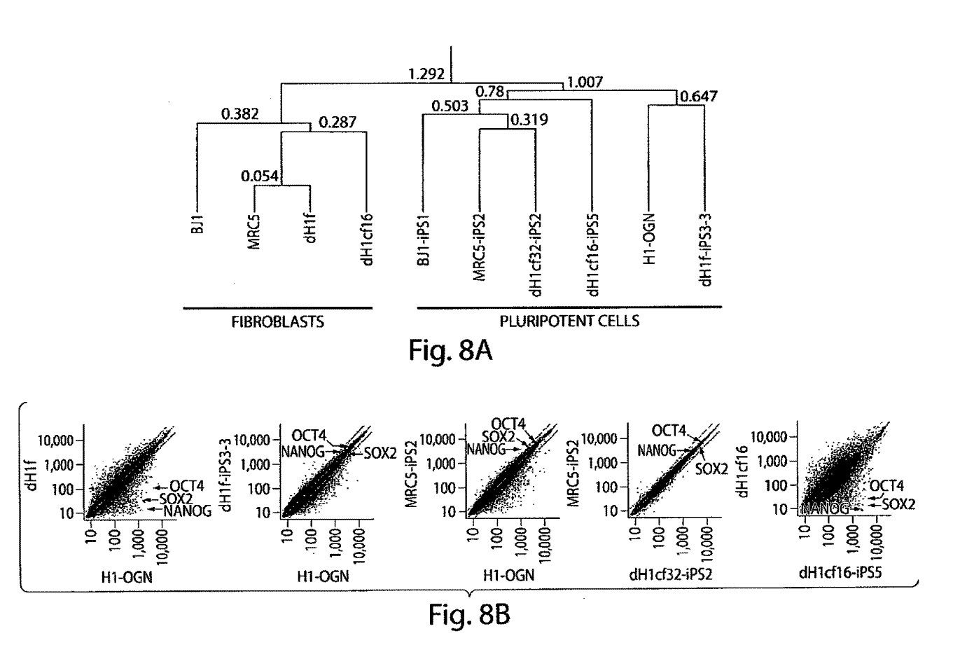

Reprogramming of somatic cells is accompanied by demethylation of promoters of critical pluripotency genes (Cowan et al., 2005; Tada et al., 2001). Therefore, we performed bisulphite sequencing to determine the extent of methylation at the OCT4 and NANOG gene promoters for two parental cell lines and their reprogrammed ES-cell-like derivatives. As expected, H1-OGN human ES cells were predominantly demethylated at the OCT4 and NANOG promoters. In contrast, the dH1f fibroblasts showed prominent methylation at these loci, consistent with transcriptional silencing in these differentiated cells. The ES-cell-like derivatives dH1f-iPS1-1 and dH1cf32-iPS2 revealed prominent demethylation, comparable to the state of these loci in H1-OGN human ES cells (FIG. 7, top). Similar data were obtained for MRC5 fetal lung fibroblasts, which showed prominent methylation of OCT4 and NANOG loci, whereas analysis of the ES-cell-like derivatives MRC5-iPS2 and MRC5-iPS19 revealed prominent demethylation (FIG. 7, bottom). These data are consistent with epigenetic remodeling of the OCT4 and NANOG promoters after retroviral infection, culture and selection for colonies with an ES-cell-like morphology.

Whereas expression analysis of a subset of genes by RT-PCR was consistent with reactivation of genes associated with pluripotency of human ES cells (FIG. 6), we performed global messenger RNA expression analysis on H1-OGN cells, parental fibroblast cells and their reprogrammed ES-cell-like derivatives. Clustering analysis revealed a high degree of similarity among the reprogrammed ES cell-like derivatives (dH1f-iPS3-3, dH1cf16-iPS5, dH1cf32-iPS2, MRC5-iPS2 and BJ1-iPS1), which clustered together with the H1-OGN ES cells and were distant from the parental somatic cells, as determined by Pearson correlation (FIG. 8A). The differentiated dH1f and dH1cf derivatives of the H1-OGN human ES cells clustered tightly with the MRC5 fetal lung fibroblasts (FIG. 8A), suggesting their close resemblance to fetal fibroblasts. Analysis of scatter plots similarly shows a tighter correlation between reprogrammed somatic cells (dH1f-iPS3-3, MRC5-iPS2) and human ES cells (H1-OGN) than between differentiated fibroblasts (dH1f) and human ES cells (H1-OGN) or differentiated fibroblasts (dH1cf16) and their reprogrammed derivative (dH1cf16-iPS5) (FIG. 8B). Different lines of reprogrammed somatic cells are particularly well correlated (MRC5-iPS2 versus dH1cf32-iPS2) (FIG. 8B). Therefore, our data indicate that the cells reprogrammed from somatic sources are highly similar to embryo-derived human ES cells at the global transcriptional level.

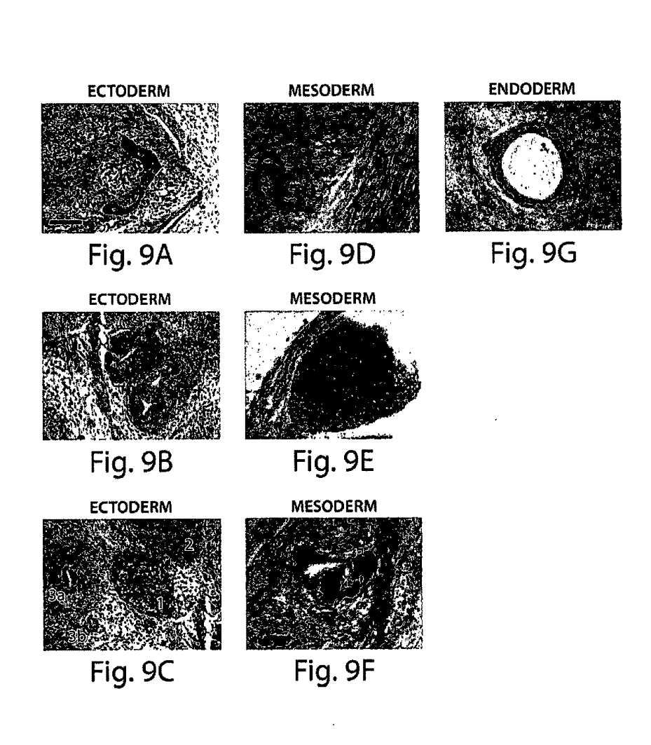

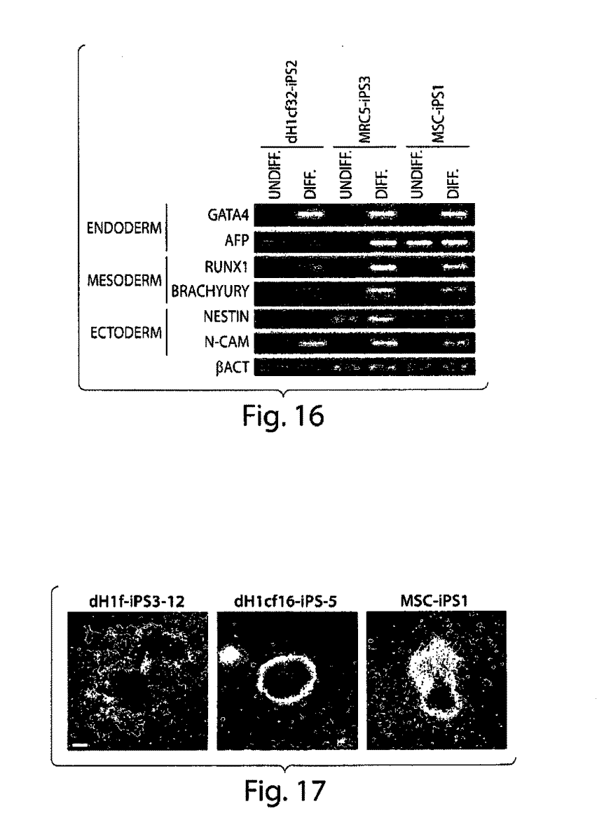

Human ES cells will form teratoma-like masses after cell injection into immunodeficient mice, an assay that has become the accepted standard for demonstrating their developmental pluripotency (Adewumi et al., 2007; Lensch et al., 2007; Lensch and Ince, 2007). We injected the human ES-cell-like cells derived from dH1f and dH1cf fibroblasts into Rag2^{-/-}/γc^{-/-} mice, and observed formation of well-encapsulated cystic tumours that harboured differentiated elements of all three primary embryonic germ layers (FIG. 9 and FIG. 15). The human ES-cell-like cells derived from dH1f, dH1cf, MRC5 and MSCs differentiated in vitro into embryoid bodies, and RT-PCR of differentiated cells showed marker gene expression for all three embryonic germ layers: GATA4 (endoderm), NCAM (ectoderm) and Brachyury and RUNX1 (mesoderm; FIG. 16). Some embryoid bodies manifest spontaneous beating, evidence of the formation of contractile cardiomyocytes with pacemaker activity (data not shown). We dissociated embryoid bodies from human ES-cell-like cells derived from dH1f, dH1cf and MSCs and plated cells in methylcellulose supplemented with haematopoietic cytokines, and detected robust formation of myeloid and erythroid colonies (FIG. 17). Taken together, our analysis of the selected derivatives of the retrovirally infected cells suggests restoration of pluripotency. Hence, consistent with the precedent in the mouse, we labelled these cells human induced pluripotent stem (iPS) cells.

TABLE 2A
Primer sets for QRT-PCR reactions

| | Forward | Reverse |
|---|---|---|
| Gene | sequence | sequence |
| ACTB | TGAAGTGTGA | GGAGGAGCAA |
| | CGTGGACATC | TGATCTTGAT |
| | (SEQ ID NO: 30) | (SEQ ID NO: 31) |
| OCT4 | AGCGAACCAG | TTACAGAACC |
| | TATCGAGAAC | ACACTCGGAC |
| | (SEQ ID NO. 32) | (SEQ ID NO: 33) |
| SOX2 | AGCTACAGCA | GGTCATGGAG |
| | TGATGCAGGA | TTGTACTGCA |
| | (SEQ ID NO: 34) | (SEQ ID NO: 35) |
| NANOG | TGAACCTCAG | TGGTGGTAGG |
| | CTACAAACAG | AAGAGTAAAG |
| | (SEQ ID NO: 36) | (SEQ ID NO: 37) |
| MYC | ACTCTGAGGA | TGGAGACGTG |
| | GGAACAAGAA | GCACCTCTT |
| | (SEQ ID NO: 38) | (SEQ ID NO: 39) |
| KLF4 | TCTCAAGGCA | TAGTGCCTGG |
| | CACCTGCGAA | TCAGTTCATC |
| | (SEQ ID NO: 40) | SEQ ID NO: 41) |
| hTERT | TGTGCACCAA | GCGTTCTTGG |
| | CATCTACAAG | CTTTCAGGAT |
| | (SEQ ID NO: 42) | (SEQ ID NO: 43) |
| REX1 | TCGCTGAGCT | CCCTTCTTGA |
| | GAAACAAATG | AGGTTTACAC |
| | (SEQ ID NO: 44) | (SEQ ID NO: 45) |
| GDF3 | AAATGTTTGT | TCTGGCACAG |
| | GTTGCGGTCA | GTGTCTTCAG |
| | (SEQ ID NO: 46) | (SEQ ID NO: 47) |
| OCT4 | CCTCACTTCA | CAGGTTTTCT |
| endo | CTGCACTGTA | TTCCCTAGCT |
| | (SEQ ID NO: 48) | (SEQ ID NO: 49) |
| OCT 4 | CCTCACTTCA | CCTTGAGGTA |
| transgene | CTGCACTGTA | CCAGAGATCT |
| | (SEQ ID NO: 50) | (SEQ ID NO: 51) |
| SOX 2 | CCCAGCAGAC | CCTCCCATTT |
| endo | TTCACATGT | CCCTCGTTTT |
| | (SEQ ID NO: 52) | (SEQ ID NO: 53) |
| SOX2 | CCCAGCAGAC | CCTTGAGGTA |
| transgene | TTCACATGT | CCAGAGATCT |
| | (SEQ ID NO: 54) | (SEQ ID NO: 55) |
| MYC | TGCCTCAAAT | GATTGAAATTC |
| endo | TGGACTTTGG | TGTGTAACTGC |
| | (SEQ ID NO: 56) | (SEQ ID NO: 57) |
| MYC | TGCCTCAAAT | CGCTCGAGGT |
| transgene | TGGACTTTGG | TAACGAATT |
| | (SEQ ID NO: 58) | (SEQ ID NO: 59) |
| KLF4 | GATGAACTGA | GTGGGTCATA |
| endo | CCAGGCACTA | TCCACTGTCT |
| | (SEQ ID NO: 60) | (SEQ ID NO: 61) |
| KLF4 | GATGAACTGA | CCTTGAGGTA |
| transgene | CCAGGCACTA | CCAGAGATCT |
| | (SEQ ID NO: 62) | (SEQ ID NO: 63) |
| RUNX1 | CCCTAGGGGA | TGAAGCTTTT |
| | TGTTCCAGAT | CCCTCTTCCA |
| | (SEQ ID NO: 64) | (SEQ ID NO: 65) |
| AFP | AGCTTGGTGG | CCCTCTTCAG |
| | TGGATGAAAC | CAAAGCAGAC |
| | (SEQ ID NO: 66) | (SEQ ID NO: 67) |
| GATA4 | CTAGACCGTG | TGGGTTAAGT |
| | GGTTTTGCAT | GCCCCTGTAG |
| | (SEQ ID NO: 68) | (SEQ ID NO: 69) |
| BRACHYURY | ACCCAGTTCA | CAATTGTCAT |
| | TAGCGGTGAC | GGGATTGCAG |
| | (SEQ ID NO: 70) | (SEQ ID NO: 71) |
| NCAM | ATGGAAACTCTAT | TAGACCTCATACT |
| | TAAAGTGAACCTG | CAGCATTCCAGT |
| | (SEQ ID NO: 72) | (SEQ ID NO: 73) |
| NESTIN | GCGTTGGAAC | TGGGAGCAAA |
| | AGAGGTTGGA | GATCCAAGAC |
| | (SEQ ID NO: 74) | (SEQ ID NO: 75) |

TABLE 2B

| | Forward | Reverse | Sequenceing |
|---|---|---|---|
| Gene | primer | primer | primer |
| SBDS | GCAAATGGTAAAGG | AAGAAAATATCTGA | AAAGACCTCGATGA |
| | CAAATACGG | CGTTTACAACATCT | AGTT |
| | (SEQ ID NO: 76) | AA | (SEQ ID NO: 78) |
| | | (SEQ ID NO: 77) | |
| HD | AGGTTCTGCTTTTAC | CGGCTGAGGAAGCT | AGGTTCTGCTTTTAC |
| | CTG | GAGGA | CTG |
| | (SEQ ID NO: 79) | (SEQ ID NO: 80) | (SEQ ID NO: 81) |
| ADA | CATGACTAGGATGG | CCTGTTATAAAGGG | CATGACTAGGATGG |
| | TTCA | CCTG | TTCA |
| | (SEQ ID NO: 82) | (SEQ ID NO: 83) | (SEQ ID NO: 84) |
| GBA | TGTGTGCAAGGTCC | ACCACCTAGAGGGG | TAGCTACTAAGGAA |
| | AGGATCAG | AAAGTG | TGTG |
| | (SEQ ID NO: 85) | (SEQ ID NO: 86) | (SEQ ID NO: 87) |

### Conclusions

We observed that differentiated fibroblast derivatives of human ES cells, primary fetal tissues (lung, skin), neonatal fibroblasts and adult fibroblasts and MSCs can be reprogrammed to pluripotency using the same four genes (OCT4, SOX2, KLF4 and MYC) that enable derivation of iPS cells from embryonic and adult fibroblasts in the mouse. When we eliminated single genes from the four-factor retroviral cocktail, we found that only OCT4 and SOX2 were essential, whereas MYC and KLF4 enhanced the efficiency of colony formation (Table 1). As a significant percentage of mice carrying iPS cells develop tumours (Okita et al., 2007), eliminating these potentially oncogenic factors would be imperative before consideration of any clinical intervention with iPS cells. Taken together, our data demonstrate that OCT4, SOX2 and either MYC or KLF4 seem to be sufficient to induce reprogramming in human cells. Other combinations of factors, including novel factors, may also promote reprogramming, and indeed NANOG and LIN28 have been shown to complement OCT4 and SOX2 in reprogramming (Yu et al., 2007).

Our results establish the feasibility of reprogramming of human primary cells with defined factors, and furthermore we provide a method for obtaining, culturing and reprogramming dermal fibroblasts from adult research subjects, which should allow the establishment of human pluripotent cells in culture from patients with specific diseases for use in research.

### Example 3

### Introduction

Human embryonic stem cells isolated from excess embryos from in vitro fertilization clinics represent an immortal propagation of pluripotent cells that theoretically can generate any cell type within the human body (Lerou et al., 2008; Murry and Keller, 2008). Human embryonic stem cells allow investigators to explore early human development through in vitro differentiation, which recapitulates aspects of normal gastrulation and tissue formation. Embryos shown to carry genetic diseases by virtue of preimplantation genetic diagnosis (PGD; genetic analysis of single blastomeres obtained by embryo biopsy) can yield stem cell lines that model single gene disorders (Verlinsky et al., 2005), but the vast majority of diseases that show more complex genetic patterns of inheritance are not represented in this pool.

A tractable method for establishing immortal cultures of pluripotent stem cells from diseased individuals would not only facilitate disease research, but also lay a foundation for producing autologous cell therapies that would avoid immune rejection and enable correction of gene defects prior to tissue reconstitution. One strategy for producing autologous, patient-derived pluripotent stem cells is somatic cell nuclear transfer (NT). In a proof of principle experiment, NT-ES cells generated from mice with genetic immunodeficiency were used to combine gene and cell therapy to repair the genetic defect (Rideout et al., 2002). To date, NT has not proven successful in the human, and given the paucity of human oocytes, is destined to have limited utility. In contrast, introducing a set of transcription factors linked to pluripotency can directly reprogram human somatic cells to produce induced pluripotent stem (iPS) cells, a method that has been achieved by several groups worldwide (Lowry et al., 2008; Park et al., 2008b; Takahashi et al., 2007; Yu et al., 2007). Given the robustness of the approach, direct reprogramming promises to be a facile source of patient-derived cell lines. Such lines would be immediately valuable for medical research, but current methods for reprogramming require infecting the somatic cells with multiple viral vectors, thereby precluding consideration of their use in transplantation medicine at this time.

Human cell culture is an essential complement to research with animal models of disease. Murine models of human congenital and acquired diseases are invaluable but provide a limited representation of human pathophysiology. Murine models do not always faithfully mimic human diseases, especially for human contiguous gene syndromes such as trisomy 21 (Down syndrome or DS). A mouse model for the DS critical region on distal human chromosome 21 fails to recapitulate the human cranial abnormalities commonly associated with trisomy 21 (Olson et al., 2004). Orthologous segments to human chromosome 21 are present on mouse chromosomes 10 and 17 and distal human chromosome 21 corresponds to mouse chromosome 16 where trisomy 16 in the mouse is lethal (Nelson and Gibbs, 2004). Thus, a true murine equivalent of human trisomy 21 does not exist. Murine strains carrying the same genetic deficiencies as the human bone marrow failure disease Fanconi anemia demonstrate DNA repair defects consistent with the human condition (e.g. (Chen et al., 1996), yet none develop the spontaneous bone marrow failure that is the hallmark of the human disease.

For cases where murine and human physiology differ, disease-specific pluripotent cells capable of differentiation into the various tissues affected in each condition could undoubtedly provide new insights into disease pathophysiology by permitting analysis in a human system, under controlled conditions in vitro, using a large number of genetically-modifiable cells, and in a manner specific to the genetic lesions in each—whether known or unknown. Here, we report the derivation of human iPS cell lines from patients with a range of human genetic diseases.

### Methods

Somatic cell culture, isolation and culture of iPS cells Fibroblasts from patients with ADA-SCID (ADA, GM01390), Gaucher disease (GD, GM00852), Duchenne type muscular dystrophy (DMD, GM04981; DMD2, GM05089), Becker type muscular dystrophy (BMD, GM04569), Down syndrome (DS1, AG0539A), Parkinson disease (PD, AG20446), juvenile (Type I) diabetes mellitus (JDM, GM02416), and Huntington disease (HD, GM04281; HD2, GM01187) were obtained from Coriell. Fibroblasts from patients with Down syndrome (DS2, DLL54) and normal fetal skin fibroblasts (Detroit 551) were purchased from ATCC. Bone marrow mesenchymal cells from SBDS patient (SBDS, DF250) has been described (Austin et al., 2005). Cells were grown in alpha-MEM containing 10% inactivated fetal serum (IFS), 50 U/ml penicillin, 50 mg/ml streptomycin, and 1 mM L-glutamine. Retroviruses expressing OCT4, SOX2, KLF4, and MYC were pseudotyped in VSVg and used to infect 1×10⁵ cells in one well of a six-well dish. iPS cells were isolated as described previously (Park et al., 2008b). iPS colonies were maintained in hES medium (80% DMEM/F12, 20% KO Serum Replacement, 10 ng/ml bFGF, 1 mM L-glutamine, 100 μM nonessential amino acids, 100 μM 2-mercaptoethanol, 50 U/ml penicillin, and 50 mg/ml streptomycin).
Characterization of genetic defects in iPS cells Genomic DNA was isolated from cells using DNeasy kit (Qiagen). PCR reactions were performed using 50 ng of genomic DNA with primers corresponding to the mutated regions of genes responsible for each condition (ADA-SCID, Gaucher disease, SBDS (Calado et al., 2007), and Huntington disease). Primer sequences are provided in Table 2B. PCR products were resolved via agarose gels, purified and sequenced, or cloned into the TOPO vector (Invitrogen) for sequencing. The number of CAG repeats in the HD gene was determined by amplifying the 5′ end of the huntington gene by PCR and sequencing. The deletion of exons within the dystrophin gene in DMD-iPS cells and BMD-iPS cells was determined by PCR using Chamberlain or Beggs' multiplex primer sets (Beggs et al., 1990; Chamberlain et al., 1988).
Karyotype analysis Chromosomal studies including karyotype of trisomy 21 in DS1-iPS and DS2-iPS10 cells were performed at the Cytogenetics Core of the Dana-Farber/Harvard Cancer Center or Cell Line Genetics using standard protocols for high-resolution G-banding.
Fingerprinting analysis 50 ng of genomic DNA was used to amplify across discrete genomic intervals containing highly variable numbers of tandem repeats (VNTR). PCR products were resolved in 3% agarose gels to examine the differential amplicon mobility for each primer set: D10S1214, repeat (GGAA)n, average heterozygosity 0.97; D17S1290, repeat (GATA)n, average heterozygosity 0.84; D7S796, repeat (GATA)n, average heterozygosity 0.95; and D21S2055, repeat (GATA)n, average heterozygosity 0.88 (Invitrogen).
Immunohistochemistry and AP staining of iPS cells iPS cells grown on feeder cells were fixed in 4% paraformaldehyde for 20 min, permeabilized with 0.2% Triton X-100 for 30 minutes, and blocked in 3% BSA in PBS for 2 hours. Cells were incubated with primary antibody overnight at 4° C., washed, and incubated with Alexa Fluor (Invitrogen) secondary antibody for 3 hours. SSEA-3, SSEA-4, TRA 1-60, TRA 1-81 antibodies were obtained from Millipore. OCT3/4 and NANOG antibodies were obtained from Abcam. Alkaline phosphatase staining was done per the manufacturer's recommendations (Millipore).
Analysis of gene expression Total RNA was isolated from iPS cells using an RNeasy kit (Qiagen) according to the manufacturer's protocol. 0.5 μg of RNA was subjected to the RT reaction using Superscript II (Invitrogen). Quantitative PCR was performed with Brilliant SYBR Green Master MiX in Stratagene MX3000P machine using previously described primers (Park et al., 2008b). Semi-quantitative PCR was performed to look at the expression of total, endogenous and recombinant pluripotency genes, and genes representing the three embryonic germ layers using primers described previously and in Table 2A.

Differentiation of iPS cells iPS cells were washed with DMEM/F12, treated with collagenase for 10 min, and collected by scraping. Colonies were washed once with DMEM/F12, and gently resuspended in EB differentiation medium. EBs were differentiated with low-speed shaking and the medium was changed every three days. After two weeks of differentiation, EBs were dissociated and plated in MethoCult (Stem Cell Technologies).

Teratoma formation from iPS cells iPS cells were washed with DMEM/F12, treated with collagenase for 10 min at room temperature, scraped using glass pipette, and collected by centrifugation. Cells were washed once with DMEM/F12, and mixed with Matrigel (BD Biosciences) and collagen (Sigma). 2×10⁶ cells were intramuscularly injected into immune deficient Rag2^{-/-}/γC^{-/-} mice. After 6 weeks of injection, teratomas were dissected, rinsed once with PBS, and fixed in 10% formalin. Embedding in paraffin, sectioning of tissue, and Hematoxylin/Eosin staining were performed by the Rodent Histopathology service of the Dana Farber Cancer Institute.

### Results

Dermal fibroblasts or bone marrow-derived mesenchymal cells were obtained from patients with a prior diagnosis of a specific disease, and used to establish disease-specific lines of human iPS cells (Table 3). This initial cohort of cell lines was derived from patients with Mendelian or complex genetic disorders, including: Down syndrome (DS; trisomy 21); adenosine deaminase deficiency-related severe combined immunodeficiency (ADA-SCID); Shwachman-Bodian-Diamond syndrome (SBDS); Gaucher disease (GD) type III; Duchenne type (DMD) and Becker type (BMD) muscular dystrophy; Huntington chorea (Huntington disease; HD); Parkinson disease (PD); and juvenile-onset, type 1 diabetes mellitus (JDM).

Patient-derived somatic cells were transduced with either four (OCT4, SOX2, KLF4, and c-MYC) or three reprogramming factors (lacking c-MYC). Following two to three weeks of culture in hES cell supporting conditions, compact refractile ES-like colonies emerged amongst a background of fibroblasts, as previously described (Park et al., 2008a; Park et al., 2008b). Although our previous report used additional factors (hTERT and SV40 LT) to achieve reprogramming of adult somatic cells, we have found the four-factor cocktail to be sufficient as long as we employ a higher multiplicity of retroviral infection. Characterization of the iPS lines is presented below.

### Mutation Analysis in iPS Lines

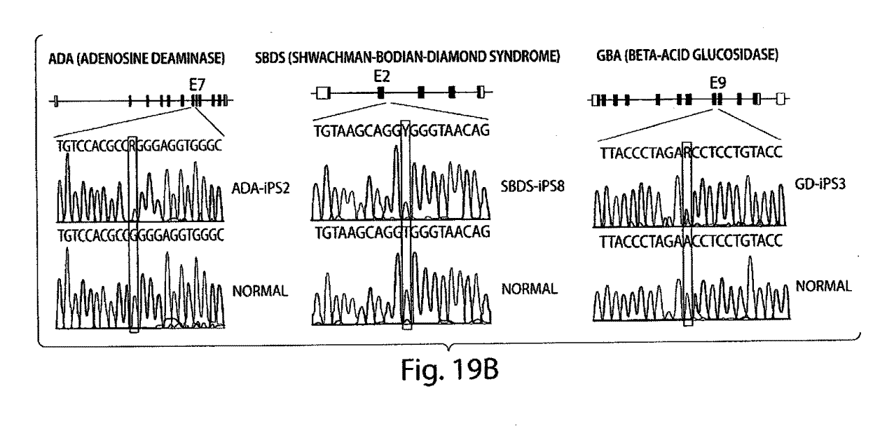

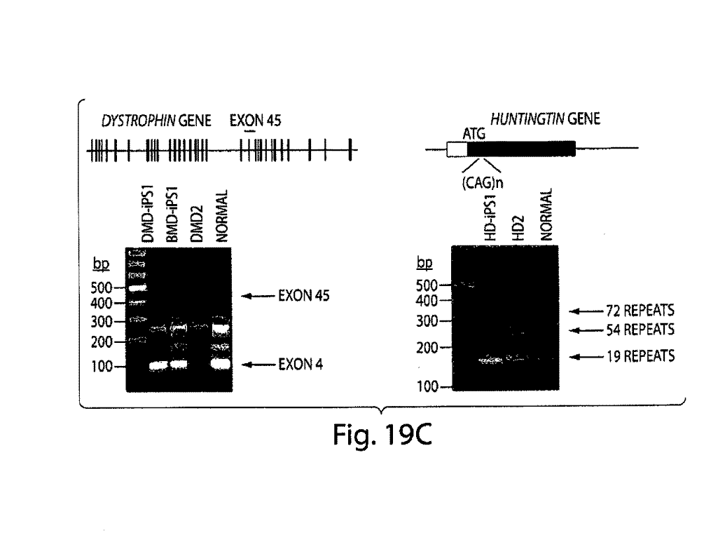

The iPS lines were evaluated to confirm, where possible, the disease-specific genotype of their parental somatic cells. Analysis of the karyotype of iPS lines derived from two individuals with Down syndrome showed the characteristic trisomy 21 anomaly (FIG. 19A). Aneuploidies such as that occurring in DS are unambiguously associated with advanced maternal age (reviewed in Antonarakis et al., 2004) and, as such, are occasionally detected in the preimplantation embryo when IVF is coupled with PGD. While it is possible that a discarded IVF embryo found to have trisomy 21 could be donated to attempt hES cell derivation, it is important to point out that many gestating DS embryos do not survive the prenatal period. Some studies place the frequency of spontaneous fetal demise (miscarriage) in DS to be above 40% (Bittles et al., 2007). Thus, the derivation of a human iPS line with trisomy 21 from an existing individual may be preferable, as such a line is most likely to harbor the complex genetic and epigenetic modifiers that favor full term gestation, and by virtue of the often lengthy medical history, will be a more informative resource for correlative clinical research.

Creation of iPS lines from patients with single-gene disorders allows experiments on disease phenotypes in vitro, and an opportunity to repair gene defects ex vivo. The resulting cells, by virtue of their immortal growth in culture, can be extensively characterized to ensure that gene repair is precise and specific, thereby reducing the safety concerns of random, viral-mediated gene therapy. Repair of gene defects in pluripotent cells provides a common platform for combined gene repair and cell replacement therapy for a variety of genetic disorders, as long as the pluripotent cells can be differentiated into relevant somatic stem cell or tissue populations.

Three diseases in our cohort of iPS cells are inherited in a classical Mendelian manner as autosomal recessive congenital disorders, and are caused by point mutations in genes essential for normal immunologic and hematopoietic function: adenosine deaminase deficiency, which causes severe combined immune deficiency (ADA-SCID) due to the absence of T-cells, B-cells, and NK-cells; Shwachman-Bodian-Diamond syndrome, a congenital disorder characterized by exocrine pancreas insufficiency, skeletal abnormalities, and bone marrow failure; and Gaucher disease type III, an autosomal recessive lysosomal storage disease characterized by pancytopenia and progressive neurological deterioration due to mutations in the acid beta-glucosidase (GBA) gene. Sequence analysis of the ADA gene in the disease-associated ADA-iPS2 line revealed a compound heterozygote: a GGG to GAA transition mutation at exon 7, causing a G216R amino acid substitution (FIG. 19B); the other allele is known to have a frame-shift deletion (-GAAGA) in exon 10 (Hirschhorn et al., 1993). The SBDS-iPS8 line harbors point mutations at the IV2+2T>C intron 2 splice donor site (FIG. 19B) and IVS3−1G>A mutation (Austin et al., 2005). Molecular analysis of the GBA gene in the Gaucher disease line revealed a 1226A>G point mutation, causing a N370S amino acid substitution (FIG. 19B); the second allele is known to have a frame-shifting insertion of a single guanine at cDNA nucleotide 84 (84GG) (Beutler et al., 1991).

Two lines were derived from dermal fibroblasts cultured from patients with muscular dystrophy. Multiplex PCR analysis with primer sets amplifying several (but not all) intragenic intervals of the dystrophin gene (Beggs et al., 1990; Chamberlain et al., 1988) revealed the deletion of exons 45-52 in the iPS cells derived from a patient with Duchenne muscular dystrophy (DMD; FIG. 19C). Despite analysis for gross genomic defects by multiplex PCR, a deletion was not detected in iPS cells derived from a patient with Becker type muscular dystrophy (BMD; FIG. 19C). As BMD is a milder form of disease, and the dystrophin gene one of the largest in the human genome, definition of the genetic lesion responsible for this condition is sometimes elusive (Prior and Bridgeman, 2005).

Given that numerous groups have pioneered the directed differentiation of neuronal subtypes, and that genetically defined ES cells from animal models of amyotrophic lateral sclerosis have revealed important insights into the pathophysiology of motor neuron deterioration (Di Giorgio et al., 2007), there is considerable interest in generating iPS lines from patients afflicted with neurodegenerative disease. We generated iPS lines from a patient with Huntington chorea (Huntington disease; HD), and verified the presence of expanded (CAG)n polyglutamine triplet repeat sequences (72) in the proximal portion of the huntington gene (FIG. 19C; (Riess et al., 1993) in one allele and 19 repeats in the other (where the normal range is 35 or less (Chong et al., 1997).

Pluripotent cell lines will likewise be valuable for studying neurodegenerative conditions with more complex genetic predisposition, as well as metabolic diseases known to have familial predispositions but for which the genetic contribution remains unexplained. We have generated lines from a patient diagnosed with Parkinson disease and another from a patient with juvenile onset (Type I) diabetes mellitus (Table 3). Given that these conditions lack a defined genetic basis, genotypic verification is impossible at this time.

### Characterization of Disease-Related iPS Lines

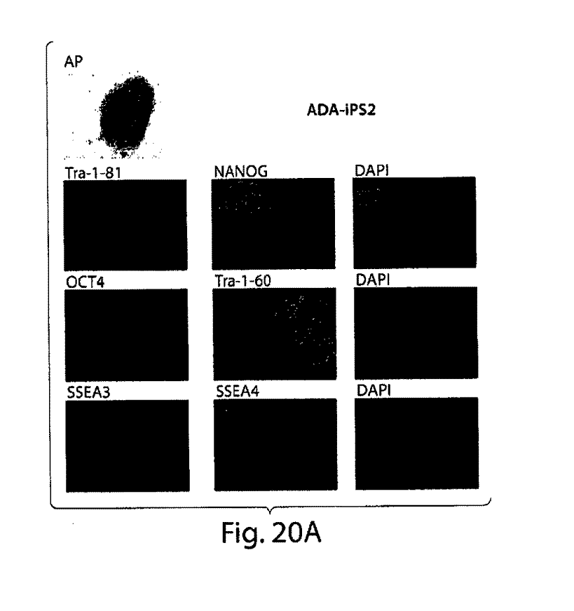

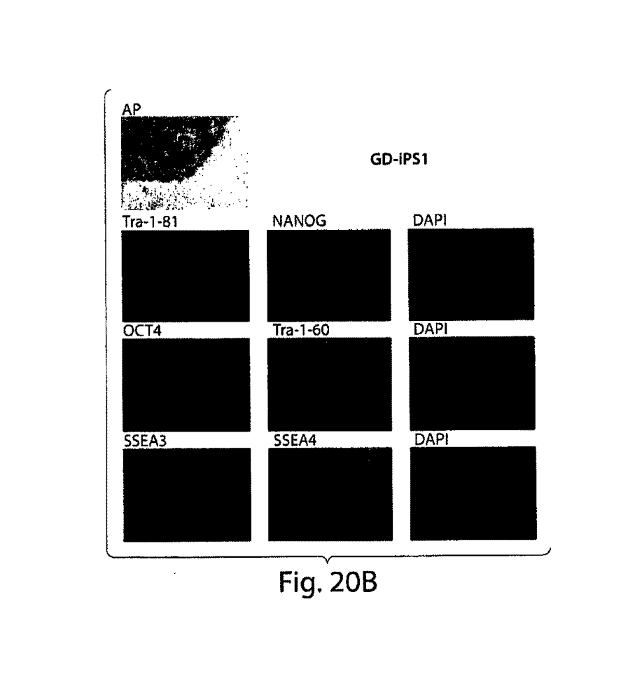

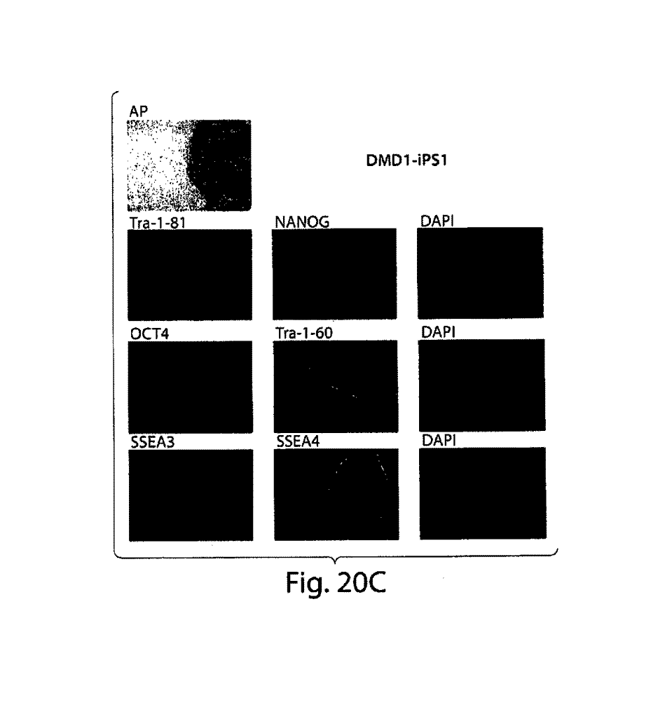

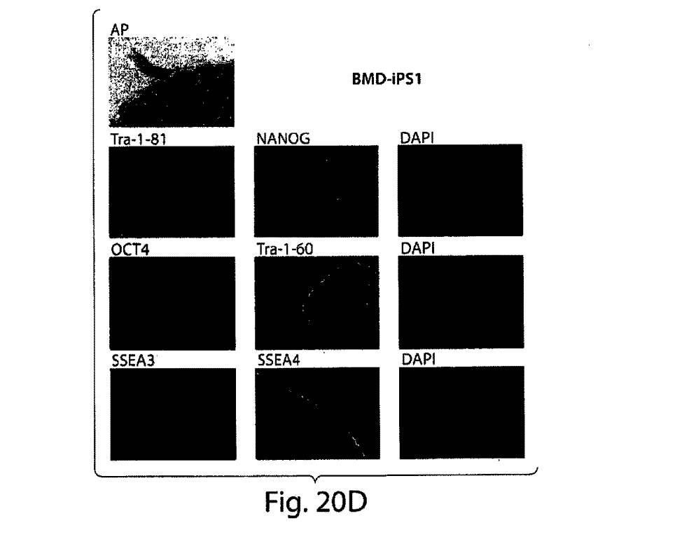

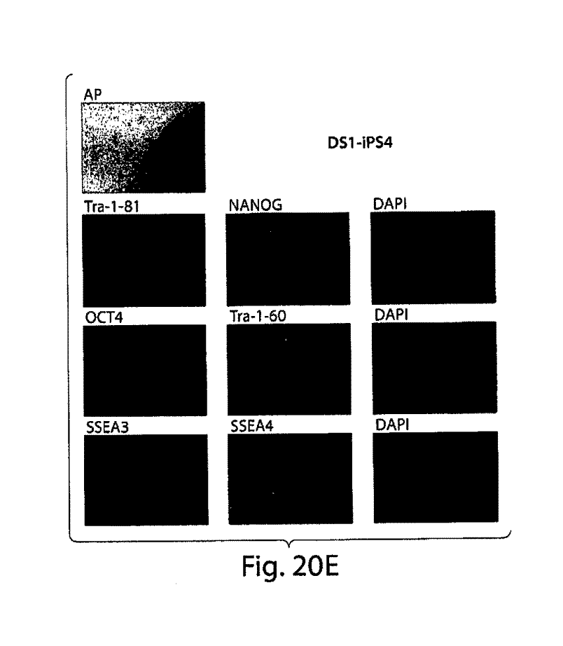

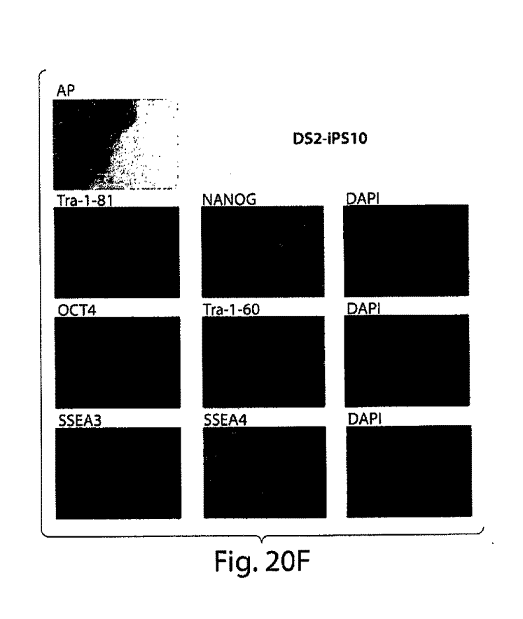

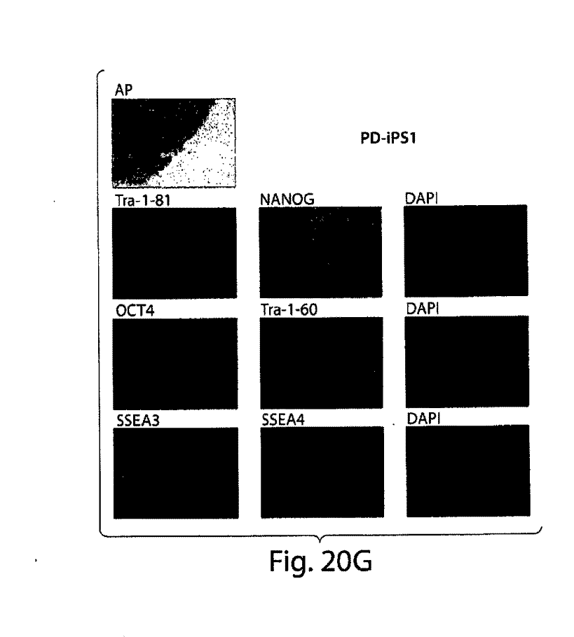

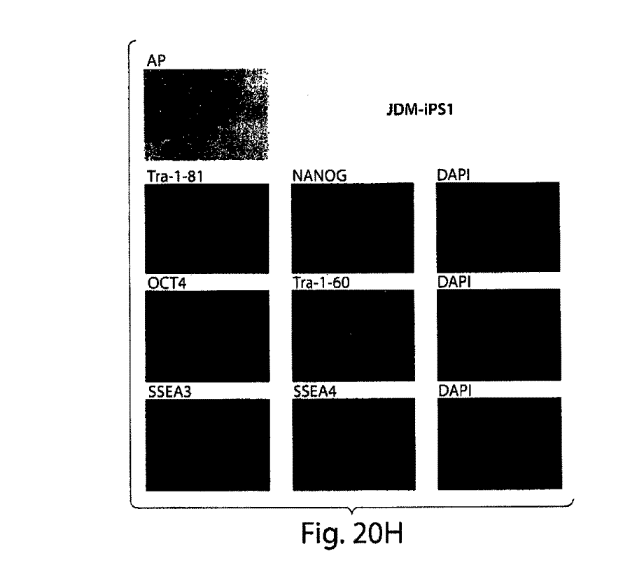

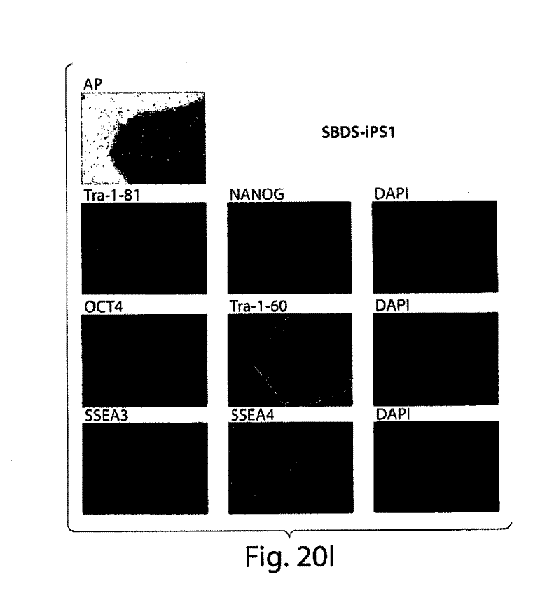

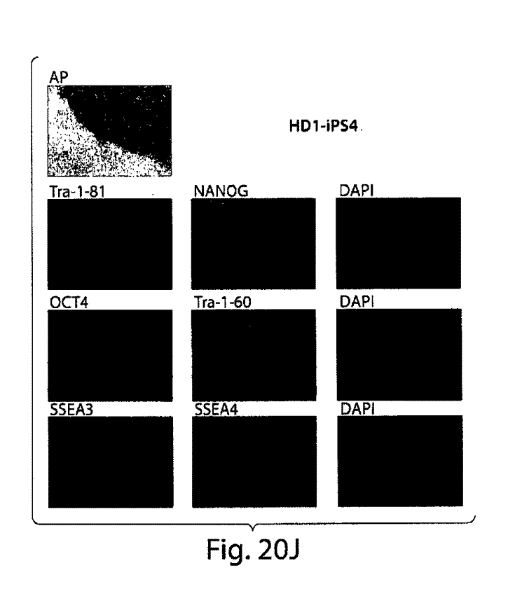

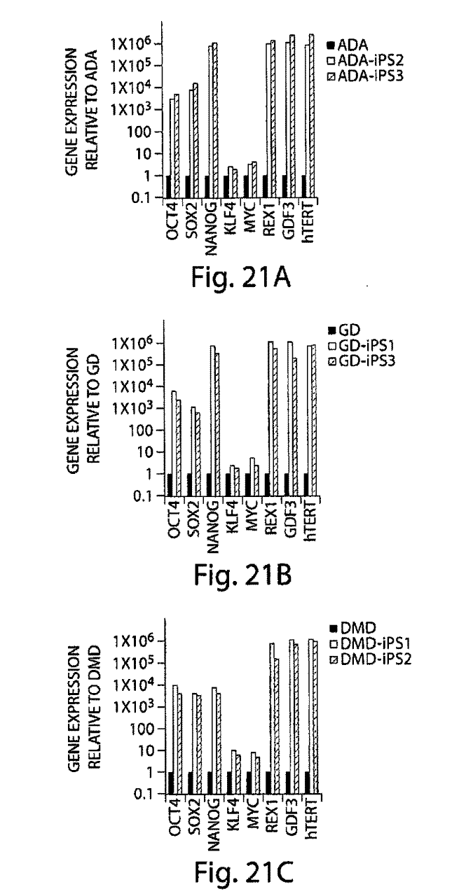

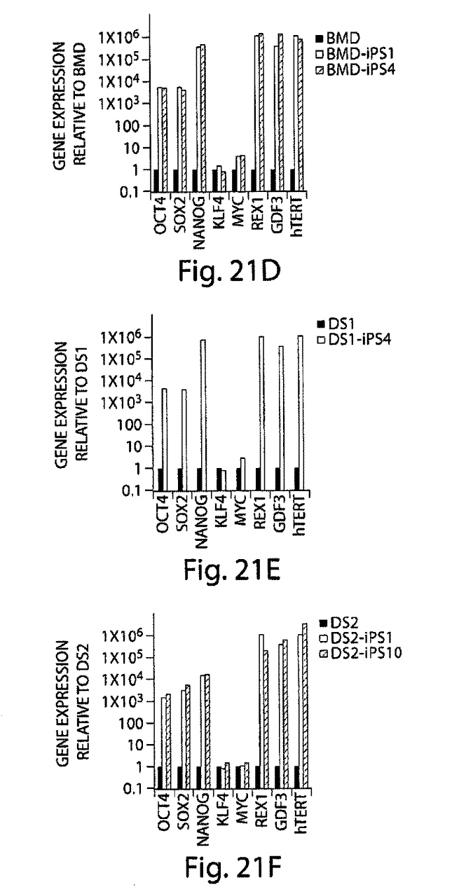

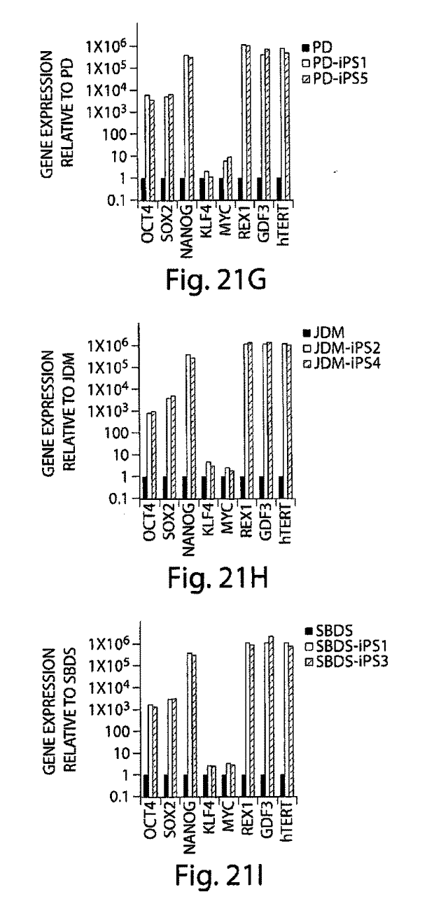

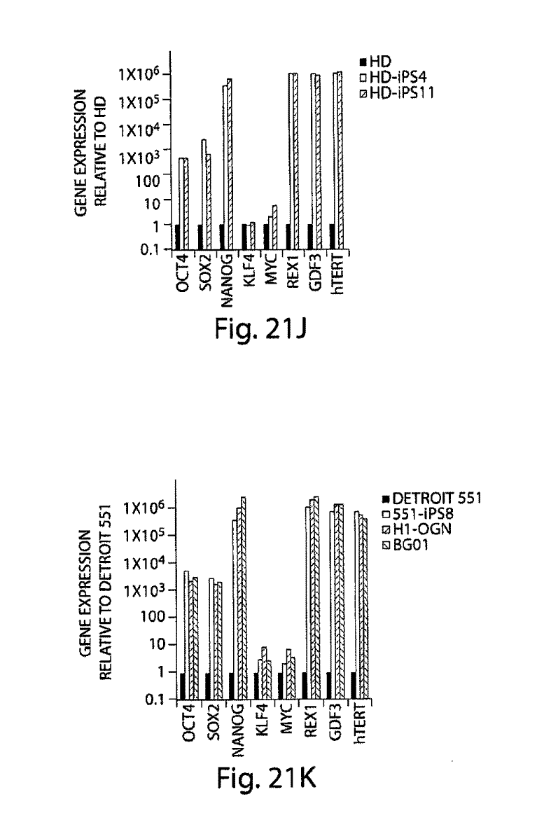

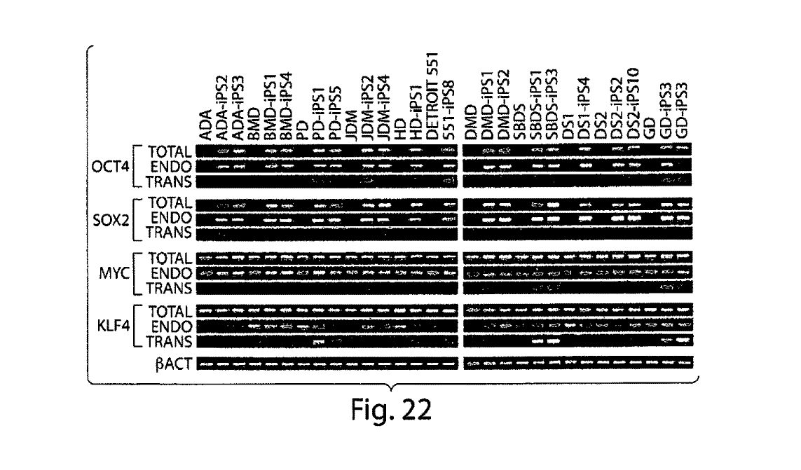

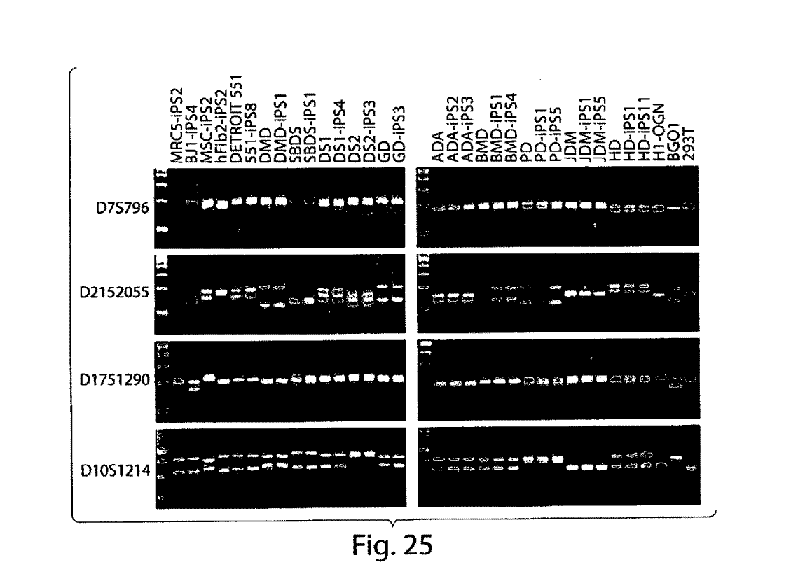

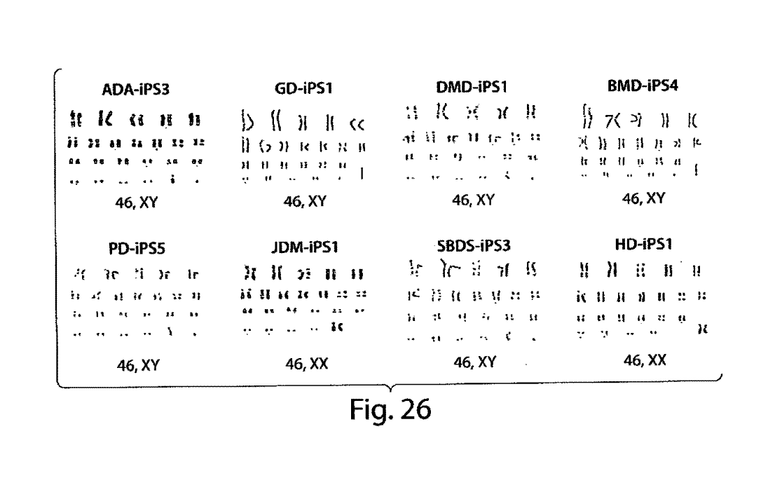

All iPS colonies, which were selected based on their morphologic resemblance to colonies of ES cells, demonstrated compact colony morphology and markers of pluripotent cells, including alkaline phosphatase (AP), Tra-1-81, Tra-1-60, OCT4, NANOG, SSEA3 and SSEA4 (FIG. 20). Quantitative RT-PCR indicated the expression of pluripotency-related genes including OCT4, SOX2, NANOG, REX1, GDF3, and hTERT regardless of the genetic condition represented within the parental somatic cells (FIG. 21; control lines are shown in panel 1). Retroviral transgenes were largely silenced in the iPS lines, with expression of the relevant reprogramming factors assumed by endogenous loci (FIG. 22), as described (Park et al., 2008b). PCR-based DNA fingerprint analysis using highly-variable number of tandem repeats (VNTR) confirmed that the iPS lines were genetically matched to their parental somatic lines, ruling out the possibility of cross-contamination from existing cultures of human pluripotent cells (FIG. 25). Also, iPS cells showed normal 46 XX, or 46 XY karyotypes (FIG. 26).

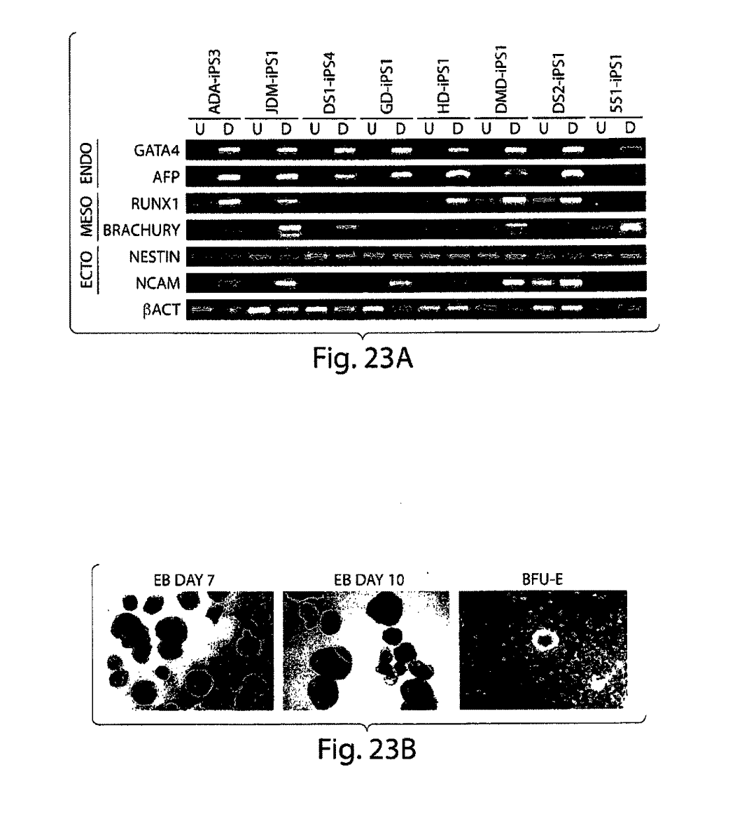

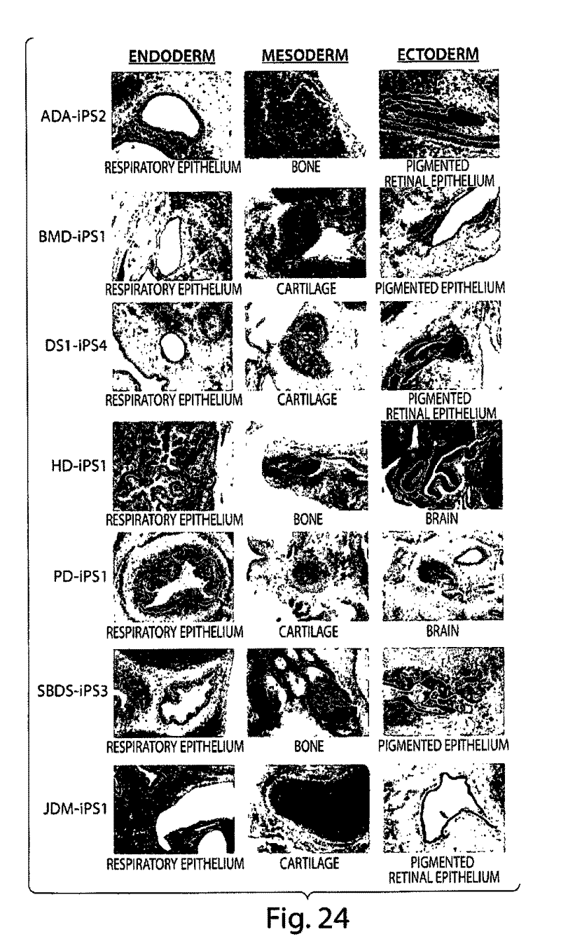

Human disease-associated iPS lines were characterized by a standard set of assays to confirm pluripotency and multi-lineage differentiation. iPS lines (n=7) were allowed to differentiate in vitro into embryoid bodies as described (Park et al., 2008b), and their potential to develop along specific lineages was confirmed by PCR for markers of all three embryonic germ layers (ectoderm, mesoderm, and endoderm; FIG. 23A). Hematopoietic differentiation of disease-specific iPS lines (n=2) produced myeloid and erythroid colony types (FIG. 23B). The ultimate standard of pluripotency for human cells is teratoma formation in immunodeficient murine hosts (Lensch et al., 2007). When injected subcutaneously into immunodeficient Rag2^{-/-}/γc^{-/-} mice, disease-specific iPS lines (n=7) produced mature, cystic masses representing all three embryonic germ layers (FIG. 24).

The technique of factor-based reprogramming of somatic cells generates pluripotent stem cell lines that are effectively immortal in culture and can be differentiated into any of a multitude of human tissues. By comparison of normal and pathologic tissue formation, and by assessment of the reparative effects of drug treatment in vitro, cell lines generated from patients offer an unprecedented opportunity to recapitulate pathologic human tissue formation in vitro, and a new technology platform for drug screening.

### Conclusion

Tissue culture of immortal cell strains from diseased patients is an invaluable resource for medical research, but is largely limited to tumor cell lines or transformed derivatives of native tissues. Here we describe the generation of induced pluripotent stem (iPS) cells from patients with a variety of genetic diseases with either Mendelian or complex inheritance that include: adenosine deaminase deficiency-related severe combined immunodeficiency (ADA-SCID), Shwachman-Bodian-Diamond syndrome (SBDS), Gaucher disease (GD) type III, Duchenne (DMD) and Becker muscular dystrophy (BMD), Parkinson disease (PD), Huntington disease (HD), juvenile-onset, type 1 diabetes mellitus (JDM), and Down syndrome (DS)/trisomy 21. Such patient-specific stem cells offer an unprecedented opportunity to recapitulate both normal and pathologic human tissue formation in vitro, thereby enabling disease investigation and drug development.

### Example 4

iPS cells have been generated using retroviral infection strategies. Retroviruses however may increase the probability of tumor development when used in vivo. To this end, various approaches are contemplated for generating iPS cells without the need for retroviruses. These include replacement of genetic reprogramming factors (as described herein) with chemical agents and the use of non-integrating viruses such as adenoviral vectors, adeno-associated viral vectors, and non-integrating lentivirus. The present invention provides an approach that involves retroviral infection but also extracts retroviral sequences (including the sequences coding for the reprogramming factors) after iPS cell generation. The invention contemplates doing this by using a Cre/lox recombination system to splice out reprogramming factor coding sequences.

The invention further contemplates the use of genetic vectors, such as retroviral vectors, that comprise coding sequences for more than one reprogramming factors. In this Example, vectors that comprise three or four reprogramming factors are provided and their ability to reprogram differentiated starting cells into iPS cells is demonstrated. These vectors preferably comprise loxP sites that flank the coding sequences for the reprogramming factors. Cre recombinase, which recombines loxP sites thereby splicing out intervening sequences, is then introduced into such cells. The Cre recombinase sequence may be introduced using another viral vector system, including for example non-integrating viruses such as adenoviruses, adeno-associated viruses, or non-integrating lentiviruses.

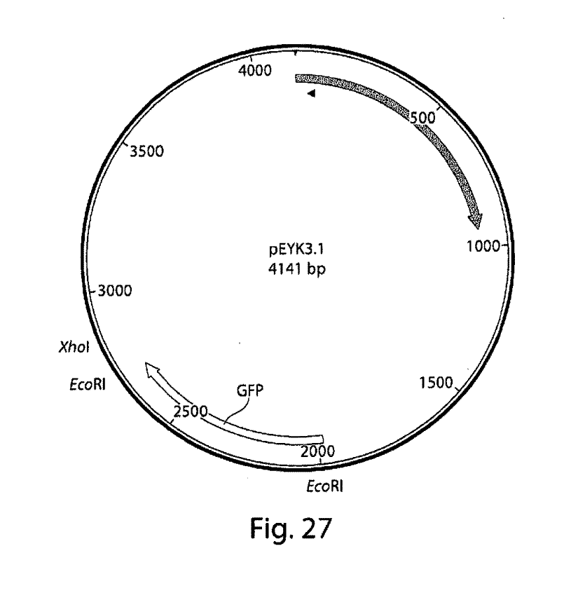

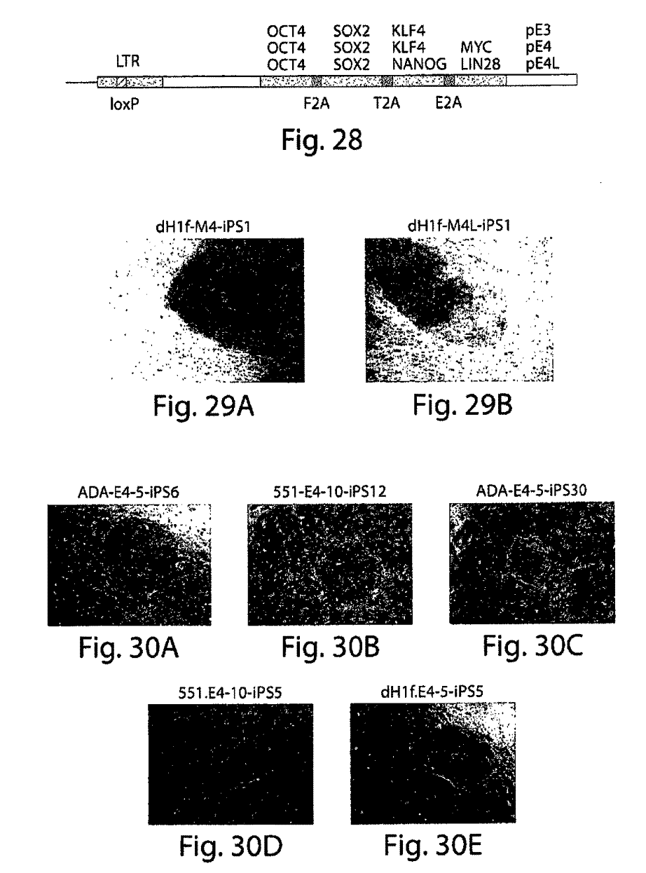

Some of the constructs generated according to the invention are derived from the pEYK3.1 vector. This vector has a single LTR that has a loxP site, which as described above, can be used to remove vector sequence including the ectopically expressed reprogramming factor sequences and the LTRs themselves. The pEYK3.1 map is shown in FIG. 27. The E3, E4 and E4L constructs were cloned into pEYK3.1 via the EcoRI and XhoI sites. In order to do this, 2 XhoI sites in the OCT4 sequence were mutated. The E3 (SEQ ID NO:19), E4 (SEQ ID NO:20) and E4L (SEQ ID NO:21) constructs replaced the GFP coding sequence in pEYK3.1. The arrangement of the various reprogramming factors and intervening viral 2A sequences are shown in FIG. 28. It to be understood that the downstream region (at the right) of these constructs will also contain a loxP site in order to allow for Cre-mediated recombination and splicing. Three and four factor encoding constructs were also generated using pMSCV-IRES-GFP (pMIG) vector. The polycistronic constructs contained in the pMIG vector are referred to herein as M3, M4 and M4L, and are identical in sequence to E3, E4 and E4L. iPS cells have been generated using either vector.

These constructs were used to reprogram a number of starting populations including ADA, dH1f and 551 cells discussed herein. Infection was performed on day 0 using 1×10⁵ cells in a 6 well plate with an MOI of 1, 2.5, 5, 10 or 20. At day 5, the cells were split and plated onto mouse embryonic fibroblasts (MEF), and at day 7 the media was replaced with hES media, as described herein.

FIGS. 29A-B show iPS colonies generated after infecting dH1f cells with retroviruses harboring pMIG-derived vectors containing M4 and M4L constructs. FIGS. 30A-E show iPS colonies generated after infecting ADA, dH1f, and 551 cells with retroviruses harboring pEYK3.1-derived vectors containing E4 constructs. iPS cell clones were generated using each of the starting populations with the E3, E4 or E4L constructs, as shown in Table 5.

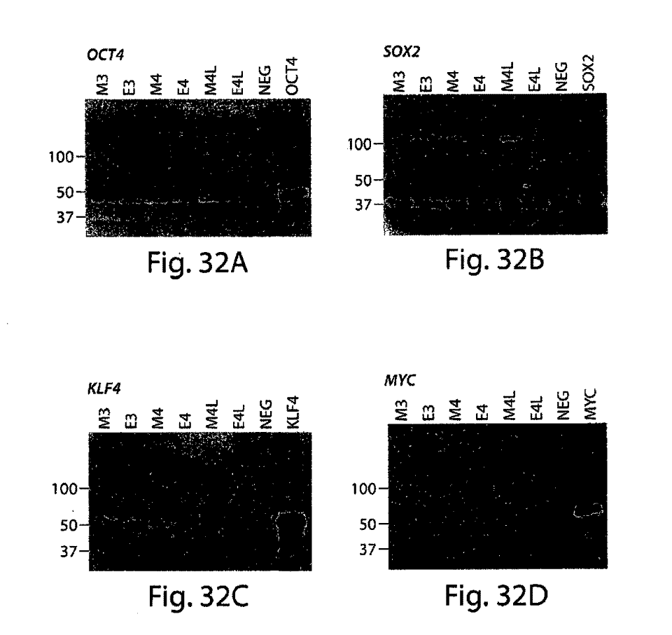

Western analysis of iPS cell clones generated using the M3, M4, M4L, E3, E4 and E4L constructs show expression of OCT4 and SOX2, as shown in FIGS. 32A and B. The same analysis also shows expression of KLF4 in iPS cell clones generated using the M3, M4, E3 and E4 constructs but not the M4L or E4L constructs, as expected, as shown in FIG. 32C. Interestingly, low or undetectable expression of MYC was found in iPS cell clones generated using M4 and E4, as shown in FIG. 32D, even though these constructs gave rise to more iPS cell colonies than did the M3 and E3 constructs which lacked MYC. This strongly suggested that MYC is being expressed, resulting in more efficient iPS cell colony generation, and that the Western analysis itself was not able to detect MYC expression.

The iPS cell clones generated using the E3, E4, E4L (and their pMIG counterparts) possess the same markers of pluripotency as do iPS cells generated by infection with multiple retroviral particles (as described herein). Representative iPS cell clones generated from dH1f cells using the M4L construct express alkaline phosphatase (AP), OCT4, NANOG, TRA-1-81, TRA-1-60, SSEA3 and SSEA4, as shown in FIGS. 31A and B.

A further analysis of the degree of retroviral construct integration into the genome of each of the generated iPS cell clones was conducted by digesting genomic DNA from each clone with EcoRI and HindIII. Southern blots were performed using a probe that binds upstream of the OCT4 sequence. FIG. 33A shows the locus of the integrated polycistron for the E4 and E4L (or M4 and M4L) constructs, with E and H respectively designating the EcoRI and HindIII sites. LTRs with loxP sites are shown as is the organization of the OCT4 (0), SOX2 (S), KLF4 (K), MYC (M), and LIN28 (L) sequences. Exemplary Southern blot data are shown in FIG. 33B. From this Figure, it can be seen that most of the iPS cell clones comprise more than one retroviral integration, with maximum number of observed integration sites on the order of about 8 per clone. No differences between clones having differing number of integration sites have been observed. Those clones with the fewest number of integration events are preferred candidates for Cre-mediated removal of the integrated retroviral sequences.

These data demonstrate that iPS cell clones can be generated efficiently using polycistronic vectors that encode all reprogramming factors of a given induction protocol.

### REFERENCES

Adewumi, O. et al. Characterization of human embryonic stem cell lines by the International Stem Cell Initiative. Nature Biotechnol. 25, 803-816 (2007).
Antonarakis, S. E., Lyle, R., Dermitzakis, E. T., Reymond, A., and Deutsch, S. (2004). Chromosome 21 and down syndrome: from genomics to pathophysiology. Nat Rev Genet 5, 725-738.
Austin, K. M., Leary, R. J., and Shimamura, A. (2005). The Shwachman-Diamond SBDS protein localizes to the nucleolus. Blood 106, 1253-1258.
Beggs, A. H., Koenig, M., Boyce, F. M., and Kunkel, L. M. (1990). Detection of 98% of DMD/BMD gene deletions by polymerase chain reaction. Hum Genet 86, 45-48.
Beutler, E., Gelbart, T., Kuhl, W., Sorge, J., and West, C. (1991). Identification of the second common Jewish Gaucher disease mutation makes possible population-based screening for the heterozygous state. Proc Natl Acad Sci USA 88, 10544-10547.
Bittles, A. H., Bower, C., Hussain, R., and Glasson, E. J. (2007). The four ages of Down syndrome. Eur J Public Health 17, 221-225.
Blelloch, R., Venere, M., Yen, J. & Ramalho-Santos, M. Generation of induced pluripotent stem cells in the absence of drug selection. Cell Stem Cell 1, 245-247 (2007).
Bodnar, A. G. et al. Extension of life-span by introduction of telomerase into normal human cells. Science 279, 349-352 (1998).
Brambrink, T., Foreman, R., Welstead, G. G., Lengner, C. J., Wernig, M., Suh, H., and Jaenisch, R. (2008). Sequential expression of pluripotency markers during direct reprogramming of mouse somatic cells. Cell Stem Cell 2, 151-159.
Calado, R. T., Graf, S. A., Wilkerson, K. L., Kajigaya, S., Ancliff, P. J., Dror, Y., Chanock, S. J., Lansdorp, P. M., and Young, N. S. (2007). Mutations in the SBDS gene in acquired aplastic anemia. Blood 110, 1141-1146.
Chamberlain, J. S., Gibbs, R. A., Ranier, J. E., Nguyen, P. N., and Caskey, C. T. (1988). Deletion screening of the Duchenne muscular dystrophy locus via multiplex DNA amplification. Nucleic Acids Res 16, 11141-11156.
Chen, M., Tomkins, D. J., Auerbach, W., McKerlie, C., Youssoufian, H., Liu, L., Gan, O., Carreau, M., Auerbach, A., Groves, T., et al. (1996). Inactivation of Fac in mice produces inducible chromosomal instability and reduced fertility reminiscent of Fanconi anaemia. Nat Genet 12, 448-451.
Chong, S. S., Almqvist, E., Telenius, H., LaTray, L., Nichol, K., Bourdelat-Parks, B., Goldberg, Y. P., Haddad, B. R., Richards, F., Sillence, D., et al. (1997). Contribution of DNA sequence and CAG size to mutation frequencies of intermediate alleles for Huntington disease: evidence from single sperm analyses. Hum Mol Genet 6, 301-309.
Cowan, C. A., Atienza, J., Melton, D. A., and Eggan, K. (2005) Science 309(5739), 1369-1373.
Deb-Rinker, P., Ly, D., Jezierski, A., Sikorska, M. & Walker, P. R. Sequential DNA methylation of the Nanog and Oct-4 upstream regions in human NT2 cells during neuronal differentiation. J. Biol. Chem. 280, 6257-6260 (2005).
Di Giorgio, F. P., Carrasco, M. A., Siao, M. C., Maniatis, T., and Eggan, K. (2007). Non-cell autonomous effect of glia on motor neurons in an embryonic stem cell-based ALS model. Nat Neurosci 10, 608-614.
Doetschman, T., Gregg, R. G., Maeda, N., Hooper, M. L., Melton, D. W., Thompson, S., and Smithies, O. (1987). Targetted correction of a mutant HPRT gene in mouse embryonic stem cells. Nature 330, 576-578.
Doetschman, T., Maeda, N., and Smithies, O. (1988). Targeted mutation of the Hprt gene in mouse embryonic stem cells. Proc Natl Acad Sci USA 85, 8583-8587.
Eischen, C. M., Roussel, M. F., Korsmeyer, S. J., and Cleveland, J. L. (2001) Mol Cell Biol 21(22), 7653-7662.
Fluckiger, A. C., Marcy, G., Marchand, M., Negre, D., Cosset, F. L., Mitalipov, S., Wolf, D., Savatier, P., and Dehay, C. (2006) Stem Cells 24(3), 547-556.
Foltynie et al. The genetic basis of Parkinson's disease. J. Neurol Neurosurg Psychiatry 2002, 73:363-370.
Freberg, C. T., Dahl, J. A., Timoskainen, S., and Collas, P. (2007) Mol Biol Cell 18(5), 1543-1553.
Grimm, S. (2004). The art and design of genetic screens: mammalian culture cells. Nat Rev Genet 5, 179-189.
Hahn, W. C. et al. Creation of human tumour cells with defined genetic elements. Nature 400, 464-468 (1999).
Hahn, W. C., Dessain, S. K., Brooks, M. W., King, J. E., Elenbaas, B., Sabatini, D. M., DeCaprio, J. A., and Weinberg, R. A. (2002) Mol Cell Biol 22(7), 2111-2123.
Hirschhorn, R., Chen, A. S., Israni, A., Yang, D. R., and Huie, M. L. (1993). Two new mutations at the adenosine deaminase (ADA) locus (Q254X and del nt1050-54) unusual for not being missense mutations. Hum Mutat 2, 320-323.
Hoffman, L. M., Hall, L., Batten, J. L., Young, H., Pardasani, D., Baetge, E. E., Lawrence, J., and Carpenter, M. K. (2005) Stem Cells 23(10), 1468-1478
Kanatsu-Shinohara, M., Inoue, K., Lee, J., Yoshimoto, M., Ogonuki, N., Miki, H., Baba, S., Kato, T., Kazuki, Y., Toyokuni, S., Toyoshima, M., Niwa, O., Oshimura, M., Heike, T., Nakahata, T., Ishino, F., Ogura, A., and Shinohara, T. (2004) Cell 119(7), 1001-1012.
Lensch, M. W., Schlaeger, T. M., Zon, L. I., and Daley, G. Q. (2007). Teratoma formation assays with human embryonic stem cells: a rationale for one type of human-animal chimera. Cell Stem Cell 1, 253-258.
Lensch, M. W. & Ince, T. A. The terminology of teratocarcinomas and teratomas. Nature Biotechnol. 25, 1211 (2007).
Lerou, P. H., Yabuuchi, A., Huo, H., Takeuchi, A., Shea, J., Cimini, T., Ince, T. A., Ginsburg, E., Racowsky, C., and Daley, G. Q. (2008). Human embryonic stem cell derivation from poor-quality embryos. Nat Biotechnol 26, 212-214.
Lowry, W. E., Richter, L., Yachechko, R., Pyle, A. D., Tchieu, J., Sridharan, R., Clark, A. T., and Plath, K. (2008). Generation of human induced pluripotent stem cells from dermal fibroblasts. Proc Natl Acad Sci USA 105, 2883-2888.
Maherali, N., Sridharan, R., Xie, W., Utikal, J., Eminli, S., Arnold, K., Stadtfeld, M., Yachechko, R., Tcheiu, J., Jaenisch, R., Plath, K., and Hochedlinger, K. (2007) Cell Stem Cell 1, 55-70.
Meissner, A., Wernig, M., and Jaenisch, R. (2007) Nat Biotechnol 25, 1177-1181.
Murry, C. E., and Keller, G. (2008). Differentiation of embryonic stem cells to clinically relevant populations: lessons from embryonic development. Cell 132, 661-680.
Nelson, D. L., and Gibbs, R. A. (2004). Genetics. The critical region in trisomy 21. Science 306, 619-621.
Nussbaum, R. L., Crowder, W. E., Nyhan, W. L., and Caskey, C. T. (1983). A three-allele restriction-fragment-length polymorphism at the hypoxanthine phosphoribosyltransferase locus in man. Proc Natl Acad Sci USA 80, 4035-4039.
Okita, K., Ichisaka, T., and Yamanaka, S. (2007) Nature, doi:10.1038/nature05934.
Olson, L. E., Richtsmeier, J. T., Leszl, J., and Reeves, R. H. (2004). A chromosome 21 critical region does not cause specific Down syndrome phenotypes. Science 306, 687-690.
Park, I. H., Lerou, P. H., Zhao, R., Huo, H., and Daley, G. Q. (2008a). Generation of human-induced pluripotent stem cells. Nat Protoc 3, 1180-1186.
Park, I. H., Zhao, R., West, J. A., Yabuuchi, A., Huo, H., Ince, T. A., Lerou, P. H., Lensch, M. W., and Daley, G. Q. (2008b). Reprogramming of human somatic cells to pluripotency with defined factors. Nature 451, 141-146.
Prior, T. W., and Bridgeman, S. J. (2005). Experience and strategy for the molecular testing of Duchenne muscular dystrophy. J Mol Diagn 7, 317-326.
Rideout, W. M., 3rd, Hochedlinger, K., Kyba, M., Daley, G. Q., and Jaenisch, R. (2002). Correction of a genetic defect by nuclear transplantation and combined cell and gene therapy. Cell 109, 17-27.
Riess, O., Noerremoelle, A., Soerensen, S. A., and Epplen, J. T. (1993). Improved PCR conditions for the stretch of (CAG)n repeats causing Huntington's disease. Hum Mol Genet 2, 637.
Stadtfeld, M., Maherali, N., Breault, D. T., and Hochedlinger, K. (2008). Defining molecular cornerstones during fibroblast to iPS cell reprogramming in mouse. Cell Stem Cell 2, 230-240.
Tada, M., Takahama, Y., Abe, K., Nakatsuji, N. & Tada, T. Nuclear reprogramming of somatic cells by in vitro hybridization with ES cells. Curr. Biol. 11, 1553-1558 (2001).
Takahashi, K., and Yamanaka, S. (2006) Cell 126(4), 663-676.
Takahashi, K., Tanabe, K., Ohnuki, M., Narita, M., Ichisaka, T., Tomoda, K., and Yamanaka, S. (2007). Induction of pluripotent stem cells from adult human fibroblasts by defined factors. Cell 131, 861-872.
Thomas, K. R., and Capecchi, M. R. (1987). Site-directed mutagenesis by gene targeting in mouse embryo-derived stem cells. Cell 51, 503-512.
Van Parijs, L. et al. Uncoupling IL-2 signals that regulate T cell proliferation, survival, and Fas-mediated activation-induced cell death. Immunity 11, 281-288 (1999).
Verlinsky, Y., Strelchenko, N., Kukharenko, V., Rechitsky, S., Verlinsky, O., Galat, V., and Kuliev, A. (2005). Human embryonic stem cell lines with genetic disorders. Reprod Biomed Online 10, 105-110.
Wakayama, T., Tabar, V., Rodriguez, I., Perry, A. C., Studer, L., and Mombaerts, P. (2001) Science 292(5517), 740-743.
Watanabe, K., Ueno, M., Kamiya, D., Nishiyama, A., Matsumura, M., Wataya, T., Takahashi, J. B., Nishikawa, S., Nishikawa, S., Muguruma, K., and Sasai, Y. (2007) Nat Biotechnol 25(6), 681-686.
Wernig, M., Meissner, A., Foreman, R., Brambrink, T., Ku, M., Hochedlinger, K., Bernstein, B. E., and Jaenisch, R. (2007) Nature, doi:10.1038/nature05944.
Xu, C., Police, S., Rao, N. & Carpenter, M. K. Characterization and enrichment of cardiomyocytes derived from human embryonic stem cells. Circ. Res. 91, 501-508 (2002).
Yu, J., Vodyanik, M. A., Smuga-Otto, K., Antosiewicz-Bourget, J., Frane, J. L., Tian, S., Nie, J., Jonsdottir, G. A., Ruotti, V., Stewart, R., et al. (2007). Induced Pluripotent Stem Cell Lines Derived from Human Somatic Cells. Science.
Zwaka, T. P., and Thomson, J. A. (2003) Nat Biotechnol 21(3), 319-321.

TABLE 3
iPS cells derived from somatic cells of patients

| Name | Disease | Defect | Coriell number | Type | Age | Gender | Cell lines |
|---|---|---|---|---|---|---|---|
| ADAf | ADA-SCID | Point mutation in adenosine | GM01390 | fibroblast | 3 M | Male | ADA-iPS2,3 |
| | | deaminase | | | | | |
| GDf | Gaucher Disease | Mutation of glucosidase, acid | GM00852 | fibroblast | 20 Y | Male | GD-iPS1, 3 |
| | | beta | | | | | |
| DMDf | Duchenne muscular | Exon 45-52 deletion | GM04981 | fibroblast | 6 Y | Male | DMD-iPS1, 2 |
| | dystrophy | | | | | | |
| BDMDf | Becker muscular | Mutation in DMD gene | GM04569 | fibroblast | 38 Y | Male | BMD-iPS1, 4 |
| | dystrophy | | | | | | |
| DS1f | Down syndrome | Trisomy 21 | AG0539A | fibroblast | 1 Y | Male | DS1-iPS4 |
| DS2f | Down syndrome | Trisomy 21 | DLL54 from ATCC | foreskin | N/A | Male | DS2-iPS1, 10 |
| | | | | fibroblast | | | |
| PDf | Parkinson's disease | N/A | AG20446) | fibroblast | 57 Y | Male | PD-iPS1, 5 |
| JDMf | Diabetes Mellitus | N/A | GM02416 | fibroblast | 42 Y | Female | JDM-iPS2, 4 |
| | Juvenile | | | | | | |
| SBDSf | Shwachman-Diamond | Mutation in SBDS gene | 1. | bone marrow | 4 M | N/A | SBDS-iPS1, 3 |
| (DF250) | syndrome | | | mesenchymal | | | |
| | | | | cells | | | |
| HDf | Huntington disease | CAG repeat in HD gene | GM04281 | fibroblast | 20 Y | Female | HD-iPS4, 11 |
| Pearson-f | Pearson syndrome | Mitochondrial deletion | GM04516 | fibroblast | 5 Y | Female | Pearson-iPS |
| | | | | | | | 1,2, and 8 |
| KSS-f | Kearns-Sayre syndrome | Mitochondrial deletion | GM06225 | fibroblast | 10 Y | Male | KSS-iPS |
| | | | | | | | 2,4, and 5 |
| Rb1f- | Retinoblastoma | Mutation in RB1 gene | GM06418 | fibroblast | 30 Y | Male | RB1-iPS1-9 |
| 30yo | | | | | | | |
| DKC-f | Dyskeratosis congenita | Mutation of Dyskerin | GM01774 | fibroblast | 7 y | Male | DKC-iPS 1 and 2 |

TABLE 4
Fibroblasts that did not give rise to iPS cells

| Name | Disease | Coriell number | Type | Age | Gender |
|---|---|---|---|---|---|
| FA1 | FANCONI ANEMIA, COMP GROUP A; FANCA | GM16632 | Skin | 13 Y | Female |
| | | | Fibroblast | | |
| FC1 | FANCONI ANEMIA, COMP GROUP C; FANCC | GM00449 | Fibroblast | 6 Y | Female |
| FG1 | FANCONI ANEMIA, COMP GROUP G; FANCG | GM02361 | Fibroblast | 14 Y | Male |
| FA2 | FCA (Fanconi A) | GM00369 | Fibroblast | 6 Y | Male |
| FC2 | FCC (Fanconi C) | GM16754 | Skin | 3 Y | Female |
| | | | Fibroblast | | |
| FD2 | FD2 (Fanconi D2) | GM16633 | Fibroblast | 7 Y | Male |

TABLE 5
iPS Cell Clone Derivation Using Polycistronic Vectors

| Starting Cell Population | Construct | No. iPS Cell Clones |
|---|---|---|
| ADA | E3 | 8 |
| ADA | E4 | 30 |
| ADA | E4L | 6 |
| 551 | E3 | Not available |
| 551 | E4 | 20 |
| 551 | E4L | 4 |
| dHf1 | E3 | 35 |
| dHf1 | E4 | 12 |
| dHf1 | E4L | 48 |

### EQUIVALENTS

It should be understood that the preceding is merely a detailed description of certain embodiments. It therefore should be apparent to those of ordinary skill in the art that various modifications and equivalents can be made without departing from the spirit and scope of the invention, and with no more than routine experimentation.

All references, patents and patent applications that are recited in this application are incorporated by reference herein in their entirety.

1. A method for producing human induced pluripotent stem cells comprising
ectopically expressing a SOX2 nucleic acid and an OCT4 nucleic acid in a differentiated human cell, and
then culturing the differentiated human cell under culture conditions and for a time sufficient for detection of a human induced pluripotent stem cell derived from the differentiated human cell.

2-11. (canceled)

12. A composition comprising
a population of human induced pluripotent stem cells produced according to the method of claim 1.

13. (canceled)

14. A method for identifying a factor that promotes production of human induced pluripotent stem cells from differentiated human cells comprising
ectopically expressing an OCT4 nucleic acid and either a SOX2 nucleic acid or a MYC nucleic acid in differentiated human cells in the presence and absence of a candidate factor,
culturing the differentiated human cells under culture conditions and for a time sufficient for detection of a human induced pluripotent stem cell derived from the differentiated human cell, and
measuring and comparing yield of human induced pluripotent stem cells produced in the presence and absence of the candidate factor,
wherein a yield of human induced pluripotent stem cells produced in the presence of the candidate factor that is greater than the yield in the absence of the candidate factor indicates a factor that promotes production of human induced pluripotent stem cells from differentiated human cells.

15-34. (canceled)

35. A method for producing human induced pluripotent stem cells from a subject comprising
ectopically expressing a SOX2 nucleic acid, an OCT4 nucleic acid and a KLF4 nucleic acid in a fibroblast obtained from the subject, and
then culturing the fibroblast under culture conditions and for a time sufficient for detection of a human induced pluripotent stem cell derived from the fibroblast,
wherein the subject has adenosine deaminase deficiency-related severe combined immunodeficiency (ADA-SCID), Gaucher disease, Duchenne type muscular dystrophy, Becker type muscular dystrophy, Down syndrome, Huntington disease, Pearson syndrome, Kearns-Sayre syndrome, retinoblastoma, Dyskeratosis congenita, Parkinson disease, juvenile type I diabetes mellitus, or Shwachman-Bodian-Diamond syndrome (SBDS).

36-59. (canceled)

60. A composition comprising a human induced pluripotent stem cell produced according to the method of claim 35.

61-73. (canceled)

74. A composition comprising a human induced pluripotent stem cell that comprises an ADA-SCID mutation, a Gaucher disease mutation, a Duchenne type muscular dystrophy mutation, a Becker type muscular dystrophy mutation, a Down syndrome mutation, a Huntington disease mutation, a Pearson syndrome mutation, a Kearns-Sayre syndrome mutation, a retinoblastoma mutation, a Dyskeratosis congenita mutation, or a Shwachman-Bodian-Diamond syndrome mutation.

75-86. (canceled)

87. A composition comprising ADA-iPS2 cell line, ADA-iPS3 cell line, GD-iPS1 cell line, GD-iPS3 cell line, DMD-iPS1 cell line, DMD-iPS2 cell line, BMD-iPS1 cell line, BMD-iPS4 cell line, DS1-iPS4 cell line, DS2-iPS1 cell line, DS2-iPS10 cell line, PD-iPS1 cell line, PD-iPS5 cell line, JDM-iPS2 cell line, JDM-iPS4 cell line, SBDS-iPS1 cell line, SBDS-iPS3 cell line, HD-iPS4 cell line, or HD-iPS11 cell line.

88-106. (canceled)

107. A method for producing human induced pluripotent stem cells comprising
introducing a polycistronic nucleic acid that comprises (a) an OCT4 nucleic acid, a SOX2 nucleic acid, and a KLF4 nucleic acid, (b) an OCT4 nucleic acid, a SOX2 nucleic acid, a KLF4 nucleic acid, and a MYC nucleic acid, or (c) an OCT4 nucleic acid, a SOX2 nucleic acid, a NANOG nucleic acid, and a LIN28 nucleic acid into a differentiated human cell,
ectopically expressing (a) the OCT4, SOX2, and KLF4 nucleic acids, (b) the OCT4, SOX2, KLF4, and MYC nucleic acids, or (c) the OCT4, SOX2, NANOG, and LIN28 nucleic acids in the differentiated human cell, and
then culturing the differentiated human cell under culture conditions and for a time sufficient for detection of a human induced pluripotent stem cell derived from the differentiated human cell.

108-119. (canceled)

120. An induced pluripotent stem cell generated according to the method of claim 107, wherein the cell comprises one or more polycistronic nucleic acids in its genome.
